(19) United States
(12) Patent Application Publication
(10) Pub. No.: US 2010/0292288 A1
(43) Pub. Date: Nov. 18, 2010

(54) CRYSTALLINE FORMS OF (R)-1-{2-[4`- (3-METHOXY-PROPANE-1- SULFONYL)-BIPHENYL-4-YL]-ETHYL}-2-METHYL-PYRROLIDINE, AND COMPOSITIONS, AND METHODS RELATED THERETO

(75) Inventors: Anthony C. Blackburn, San Diego, CA (US); John A. DeMattei, San Diego, CA (US); Ryan M. Hart, San Francisco, CA (US); Young Mi Khulman, San Diego, CA (US); Michael Mesleh, La Jolla, CA (US); Jeffrey Smith, San Diego, CA (US)

Correspondence Address: FISH & RICHARDSON P.C., P.O. BOX 1022, MINNEAPOLIS, MN, 55440-1022, US

(73) Assignee: ARENA PHARMACEUTICALS, INC., San Diego, CA (US)

(21) Appl. No.: 12/663,415

(22) PCT Filed: Jun. 6, 2008
(86) PCT No.: PCT/US2008/007144

§ 371 Date: May 24, 2010

Related U.S. Application Data
(60) Provisional application No. 60/933,791, filed on Jun. 8, 2007.
(60) Provisional application No. 61/124,527, filed on Apr. 16, 2008.

(51) Int. Cl. A61K 31/40; C07D 207/04; A61P 25/28; A61P 25/08; A61P 25/00; A61P 25/24; A61P 3/04; A61P 25/20; A61P 25/18; A61P 11/00; A61P 29/00

(52) U.S. Cl. 514/408; 548/578

(57) ABSTRACT

The present invention is directed to novel salts of (R)-1-{2-[4′-(3-methoxy-propane-1-sulfonyl)-biphenyl-4-yl]-ethyl}-2-methyl-pyrrolidine, and crystalline forms, and compositions thereof that modulate the activity of the histamine H3-receptor and are useful in the treatment of histamine H3-receptor associated disorders, such as, cognitive disorders, epilepsy, brain trauma, depression, obesity, disorders of sleep and wakefulness such as narcolepsy, shift-work syndrome, drowsiness as a side effect from a medication, maintenance of vigilance to aid in completion of tasks and the like, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, pain, dementia, Alzheimer's disease and the like.

### FIELD OF THE INVENTION

The present invention is directed to novel salts of (R)-1-{2-[4′-(3-methoxy-propane-1-sulfonyl)-biphenyl-4-yl]-ethyl}-2-methyl-pyrrolidine, and crystalline forms, and compositions thereof that modulate the activity of the histamine H3-receptor and are useful in the treatment of histamine H3-receptor associated disorders, such as, cognitive disorders, epilepsy, brain trauma, depression, obesity, disorders of sleep and wakefulness such as narcolepsy, shift-work syndrome, drowsiness as a side effect from a medication, maintenance of vigilance to aid in completion of tasks and the like, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, pain, dementia, Alzheimer's disease and the like.

### BACKGROUND OF THE INVENTION

The compound, (R)-1-{2-[4′-(3-methoxy-propane-1-sulfonyl)-biphenyl-4-yl]-ethyl}-2-methyl-pyrrolidine (Compound I, below) belongs to a class of histamine H3-receptor modulators that are useful in the treatment of histamine H3-receptor associated diseases and disorders.

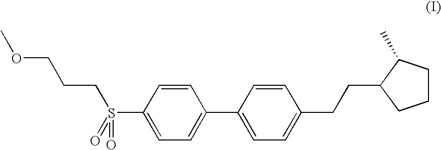

Because drug compounds having, for example, improved processability, stability, solubility, shelf life, and in vivo pharmacology, are consistently sought, there is an ongoing need for new or purer salts, hydrates, solvates, and polymorphic crystal forms of drug molecules. The salts of crystalline forms of (R)-1-{2-[4′-(3-methoxy-propane-1-sulfonyl)-biphenyl-4-yl]-ethyl}-2-methyl-pyrrolidine as described herein help meet one or more of these and other needs.

### SUMMARY OF THE INVENTION

One aspect of the present invention is directed to salts and crystalline forms of (R)-1-{2-[4′-(3-methoxy-propane-1-sulfonyl)-biphenyl-4-yl]-ethyl}-2-methyl-pyrrolidine selected from the group consisting of (R)-1-{2-[4′-(3-methoxy-propane-1-sulfonyl)-biphenyl-4-yl]-ethyl}-2-methyl-pyrrolidine mono-citrate; (R)-1-{2-[4′-(3-methoxy-propane-1-sulfonyl)-biphenyl-4-yl]-ethyl}-2-methyl-pyrrolidine di-citrate; (R)-1-{2-[4′-(3-methoxy-propane-1-sulfonyl)-biphenyl-4-yl]-ethyl}-2-methyl-pyrrolidine maleate; and (R)-1-{2-[4′-(3-methoxy-propane-1-sulfonyl)-biphenyl-4-yl]-ethyl}-2-methyl-pyrrolidine hydrochloride.

One aspect of the present invention is directed to (R)-1-{2-[4′-(3-methoxy-propane-1-sulfonyl)-biphenyl-4-yl]-ethyl}-2-methyl-pyrrolidine mono-citrate.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate.

One aspect of the present invention is directed to (R)-1-{2-[4′-(3-methoxy-propane-1-sulfonyl)-biphenyl-4-yl]-ethyl}-2-methyl-pyrrolidine di-citrate.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate.

One aspect of the present invention is directed to (R)-1-{2-[4′-(3-methoxy-propane-1-sulfonyl)-biphenyl-4-yl]-ethyl}-2-methyl-pyrrolidine maleate.

One aspect of the present invention is directed to a crystalline form of (R)-1-{2-[4′-(3-methoxy-propane-1-sulfonyl)-biphenyl-4-yl]-ethyl}-2-methyl-pyrrolidine maleate.

One aspect of the present invention is directed to (R)-1-{2-[4′-(3-methoxy-propane-1-sulfonyl)-biphenyl-4-yl]-ethyl}-2-methyl-pyrrolidine hydrochloride.

One aspect of the present invention is directed to a crystalline form of (R)-1-{2-[4′-(3-methoxy-propane-1-sulfonyl)-biphenyl-4-yl]-ethyl}-2-methyl-pyrrolidine hydrochloride.

One aspect of the present invention is directed to compositions comprising (R)-1-{2-[4′-(3-methoxy-propane-1-sulfonyl)-biphenyl-4-yl]-ethyl}-2-methyl-pyrrolidine mono-citrate or the crystalline form thereof.

One aspect of the present invention is directed to compositions comprising (R)-1-{2-[4′-(3-methoxy-propane-1-sulfonyl)-biphenyl-4-yl]-ethyl}-2-methyl-pyrrolidine di-citrate or the crystalline form thereof.

One aspect of the present invention is directed to compositions comprising (R)-1-{2-[4′-(3-methoxy-propane-1-sulfonyl)-biphenyl-4-yl]-ethyl}-2-methyl-pyrrolidine maleate or a crystalline form thereof.

One aspect of the present invention is directed to compositions comprising (R)-1-{2-[4′-(3-methoxy-propane-1-sulfonyl)-biphenyl-4-yl]-ethyl}-2-methyl-pyrrolidine hydrochloride or a crystalline form thereof.

One aspect of the present invention is directed to pharmaceutical compositions comprising (R)-1-{2-[4′-(3-methoxy-propane-1-sulfonyl)-biphenyl-4-yl]-ethyl}-2-methyl-pyrrolidine mono-citrate and a pharmaceutically acceptable carrier.

One aspect of the present invention is directed to pharmaceutical compositions comprising the crystalline form of (R)-1-{2-[4′-(3-methoxy-propane-1-sulfonyl)-biphenyl-4-yl]-ethyl}-2-methyl-pyrrolidine mono-citrate and a pharmaceutically acceptable carrier.

One aspect of the present invention is directed to pharmaceutical compositions comprising (R)-1-{2-[4′-(3-methoxy-propane-1-sulfonyl)-biphenyl-4-yl]-ethyl}-2-methyl-pyrrolidine di-citrate and a pharmaceutically acceptable carrier.

One aspect of the present invention is directed to pharmaceutical compositions comprising the crystalline form of (R)-1-{2-[4′-(3-methoxy-propane-1-sulfonyl)-biphenyl-4-yl]-ethyl}-2-methyl-pyrrolidine di-citrate and a pharmaceutically acceptable carrier.

One aspect of the present invention is directed to pharmaceutical compositions comprising (R)-1-{2-[4′-(3-methoxy-propane-1-sulfonyl)-biphenyl-4-yl]-ethyl}-2-methyl-pyrrolidine maleate and a pharmaceutically acceptable carrier.

One aspect of the present invention is directed to pharmaceutical compositions comprising a crystalline form of (R)-1-{2-[4′-(3-methoxy-propane-1-sulfonyl)-biphenyl-4-yl]-ethyl}-2-methyl-pyrrolidine maleate and a pharmaceutically acceptable carrier.

One aspect of the present invention is directed to pharmaceutical compositions comprising (R)-1-{2-[4′-(3-methoxy-propane-1-sulfonyl)-biphenyl-4-yl]-ethyl}-2-methyl-pyrrolidine hydrochloride and a pharmaceutically acceptable carrier.

One aspect of the present invention is directed to pharmaceutical compositions comprising a crystalline form of (R)-1-{2-[4′-(3-methoxy-propane-1-sulfonyl)-biphenyl-4-yl]-ethyl}-2-methyl-pyrrolidine hydrochloride and a pharmaceutically acceptable carrier.

In some embodiments, the present invention is directed to methods of treating a histamine H3-receptor associated disorder in an individual comprising administering to the individual in need thereof a therapeutically effective amount of (R)-1-{2-[4′-(3-methoxy-propane-1-sulfonyl)-biphenyl-4-yl]-ethyl}-2-methyl-pyrrolidine mono-citrate or a pharmaceutical composition thereof.

In some embodiments, the present invention is directed to methods of treating a histamine H3-receptor associated disorder in an individual comprising administering to the individual in need thereof a therapeutically effective amount of the crystalline form of (R)-1-{2-[4′-(3-methoxy-propane-1-sulfonyl)-biphenyl-4-yl]-ethyl}-2-methyl-pyrrolidine mono-citrate or a pharmaceutical composition thereof.

In some embodiments, the present invention is directed to methods of treating a histamine H3-receptor associated disorder in an individual comprising administering to the individual in need thereof a therapeutically effective amount of (R)-1-{2-[4′-(3-methoxy-propane-1-sulfonyl)-biphenyl-4-yl]-ethyl}-2-methyl-pyrrolidine di-citrate or a pharmaceutical composition thereof.

In some embodiments, the present invention is directed to methods of treating a histamine H3-receptor associated disorder in an individual comprising administering to the individual in need thereof a therapeutically effective amount of the crystalline form of (R)-1-{2-[4′-(3-methoxy-propane-1-sulfonyl)-biphenyl-4-yl]-ethyl}-2-methyl-pyrrolidine di-citrate or a pharmaceutical composition thereof.

In some embodiments, the present invention is directed to methods of treating a histamine H3-receptor associated disorder in an individual comprising administering to the individual in need thereof a therapeutically effective amount of (R)-1-{2-[4′-(3-methoxy-propane-1-sulfonyl)-biphenyl-4-yl]-ethyl}-2-methyl-pyrrolidine maleate or a pharmaceutical composition thereof.

In some embodiments, the present invention is directed to methods of treating a histamine H3-receptor associated disorder in an individual comprising administering to the individual in need thereof a therapeutically effective amount of a crystalline form of (R)-1-{2-[4′-(3-methoxy-propane-1-sulfonyl)-biphenyl-4-yl]-ethyl}-2-methyl-pyrrolidine maleate or a pharmaceutical composition thereof.

In some embodiments, the present invention is directed to methods of treating a histamine H3-receptor associated disorder in an individual comprising administering to the individual in need thereof a therapeutically effective amount of (R)-1-{2-[4′-(3-methoxy-propane-1-sulfonyl)-biphenyl-4-yl]-ethyl}-2-methyl-pyrrolidine hydrochloride or a pharmaceutical composition thereof.

In some embodiments, the present invention is directed to methods of treating a histamine H3-receptor associated disorder in an individual comprising administering to the individual in need thereof a therapeutically effective amount of a crystalline form of (R)-1-{2-[4′-(3-methoxy-propane-1-sulfonyl)-biphenyl-4-yl]-ethyl}-2-methyl-pyrrolidine hydrochloride or a pharmaceutical composition thereof.

In some embodiments, the present invention is directed to methods of inducing wakefulness in an individual comprising administering to said individual in need thereof a therapeutically effective amount of (R)-1-{2-[4′-(3-methoxy-propane-1-sulfonyl)-biphenyl-4-yl]-ethyl}-2-methyl-pyrrolidine mono-citrate or a pharmaceutical composition thereof.

In some embodiments, the present invention is directed to methods of inducing wakefulness in an individual comprising administering to said individual in need thereof a therapeutically effective amount of the crystalline form of (R)-1-{2-[4′-(3-methoxy-propane-1-sulfonyl)-biphenyl-4-yl]-ethyl}-2-methyl-pyrrolidine mono-citrate or a pharmaceutical composition thereof.

In some embodiments, the present invention is directed to methods of inducing wakefulness in an individual comprising administering to said individual in need thereof a therapeutically effective amount of (R)-1-{2-[4′-(3-methoxy-propane-1-sulfonyl)-biphenyl-4-yl]-ethyl}-2-methyl-pyrrolidine di-citrate or a pharmaceutical composition thereof.

In some embodiments, the present invention is directed to methods of inducing wakefulness in an individual comprising administering to said individual in need thereof a therapeutically effective amount of the crystalline form of (R)-1-{2-[4′-(3-methoxy-propane-1-sulfonyl)-biphenyl-4-yl]-ethyl}-2-methyl-pyrrolidine di-citrate or a pharmaceutical composition thereof.

In some embodiments, the present invention is directed to methods of inducing wakefulness in an individual comprising administering to said individual in need thereof a therapeutically effective amount of (R)-1-{2-[4′-(3-methoxy-propane-1-sulfonyl)-biphenyl-4-yl]-ethyl}-2-methyl-pyrrolidine maleate or a pharmaceutical composition thereof.

In some embodiments, the present invention is directed to methods of inducing wakefulness in an individual comprising administering to said individual in need thereof a therapeutically effective amount of a crystalline form of (R)-1-{2-[4′-(3-methoxy-propane-1-sulfonyl)-biphenyl-4-yl]-ethyl}-2-methyl-pyrrolidine maleate or a pharmaceutical composition thereof.

In some embodiments, the present invention is directed to methods of inducing wakefulness in an individual comprising administering to said individual in need thereof a therapeutically effective amount of (R)-1-{2-[4′-(3-methoxy-propane-1-sulfonyl)-biphenyl-4-yl]-ethyl}-2-methyl-pyrrolidine hydrochloride or a pharmaceutical composition thereof.

In some embodiments, the present invention is directed to methods of inducing wakefulness in an individual comprising administering to said individual in need thereof a therapeutically effective amount of a crystalline form of (R)-1-{2-[4′-(3-methoxy-propane-1-sulfonyl)-biphenyl-4-yl]-ethyl}-2-methyl-pyrrolidine hydrochloride or a pharmaceutical composition thereof.

In some embodiments, the present invention is directed to uses of citrate salts of (R)-1-{2-[4′-(3-methoxy-propane-1-sulfonyl)-biphenyl-4-yl]-ethyl}-2-methyl-pyrrolidine and crystalline forms thereof, as described herein, for the manufacture of a medicament for the treatment of a histamine H3-receptor associated disorder.

In some embodiments, the present invention is directed to uses of a maleate salt of (R)-1-{2-[4′-(3-methoxy-propane-1-sulfonyl)-biphenyl-4-yl]-ethyl}-2-methyl-pyrrolidine and crystalline forms thereof, as described herein, for the manufacture of a medicament for the treatment of a histamine H3-receptor associated disorder.

In some embodiments, the present invention is directed to uses of a hydrochloride salt of (R)-1-{2-[4′-(3-methoxy-propane-1-sulfonyl)-biphenyl-4-yl]-ethyl}-2-methyl-pyrrolidine and crystalline forms thereof, as described herein, for the manufacture of a medicament for the treatment of a histamine H3-receptor associated disorder.

In some embodiments, the present invention is directed to citrate salts of (R)-1-{2-[4′-(3-methoxy-propane-1-sulfonyl)-biphenyl-4-yl]-ethyl}-2-methyl-pyrrolidine and crystalline forms thereof, as described herein, for use in a method of treatment of the human or animal body by therapy.

In some embodiments, the present invention is directed to a maleate salt of (R)-1-{2-[4′-(3-methoxy-propane-1-sulfonyl)-biphenyl-4-yl]-ethyl}-2-methyl-pyrrolidine and crystalline forms thereof, as described herein, for use in a method of treatment of the human or animal body by therapy.

In some embodiments, the present invention is directed to a hydrochloride salt of (R)-1-{2-[4′-(3-methoxy-propane-1-sulfonyl)-biphenyl-4-yl]-ethyl}-2-methyl-pyrrolidine and crystalline forms thereof, as described herein, for use in a method of treatment of the human or animal body by therapy.

In some embodiments, the present invention is directed to citrate salts of (R)-1-{2-[4′-(3-methoxy-propane-1-sulfonyl)-biphenyl-4-yl]-ethyl}-2-methyl-pyrrolidine and crystalline forms thereof, as described herein, for use in a method for the treatment of a histamine H3-receptor associated disorder in the human or animal body by therapy.

In some embodiments, the present invention is directed to a maleate salt of (R)-1-{2-[4′-(3-methoxy-propane-1-sulfonyl)-biphenyl-4-yl]-ethyl}-2-methyl-pyrrolidine and crystalline forms thereof, as described herein, for use in a method for the treatment of a histamine H3-receptor associated disorder in the human or animal body by therapy.

In some embodiments, the present invention is directed to a hydrochloride salt of (R)-1-{2-[4′-(3-methoxy-propane-1-sulfonyl)-biphenyl-4-yl]-ethyl}-2-methyl-pyrrolidine and crystalline forms thereof, as described herein, for use in a method for the treatment of a histamine H3-receptor associated disorder in the human or animal body by therapy.

In some embodiments, an H3-receptor associated disorder is selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, pain, dementia and Alzheimer's disease.

One aspect of the present invention is directed to processes for preparing pharmaceutical compositions comprising admixing a compound or a crystalline form thereof, as described herein, and a pharmaceutically acceptable carrier.

One aspect of the present invention is directed to processes for preparing citrate salts of (R)-1-{2-[4′-(3-methoxy-propane-1-sulfonyl)-biphenyl-4-yl]-ethyl}-2-methyl-pyrrolidine and crystalline forms thereof.

One aspect of the present invention is directed to processes for preparing a maleate salt of (R)-1-{2-[4′-(3-methoxy-propane-1-sulfonyl)-biphenyl-4-yl]-ethyl}-2-methyl-pyrrolidine and crystalline forms thereof.

One aspect of the present invention is directed to processes for preparing a hydrochloride salt of (R)-1-{2-[4′-(3-methoxy-propane-1-sulfonyl)-biphenyl-4-yl]-ethyl}-2-methyl-pyrrolidine and crystalline forms thereof.

### BRIEF DESCRIPTION OF THE DRAWINGS

FIG. 1 depicts a powder X-ray diffraction (PXRD) pattern for the novel crystalline form of (R)-1-{2-[4′-(3-methoxy-propane-1-sulfonyl)-biphenyl-4-yl]-ethyl}-2-methyl-pyrrolidine mono-citrate (FORM 1), which was recorded using a PANalytical X'Pert Plus Powder X-Ray Diffractometer in the 2θ geometry; scanning angles 5.0°-40.0° 2θ.

FIG. 2 depicts a differential scanning calorimetry (DSC) thermogram for the novel crystalline form of (R)-1-{2-[4′-(3-methoxy-propane-1-sulfonyl)-biphenyl-4-yl]-ethyl}-2-methyl-pyrrolidine mono-citrate (FORM 1), which was recorded using a TA Instruments DSC Q1000; 25-170° C. at 10° C./min.

FIG. 3 depicts a FTIR spectrum for the novel crystalline form of (R)-1-{2-[4′-(3-methoxy-propane-1-sulfonyl)-biphenyl-4-yl]-ethyl}-2-methyl-pyrrolidine mono-citrate (FORM 1), which was recorded using a Bruker Tensor 27 FTIR spectrometer running OPUS 4.2 software.

FIG. 4 depicts a thermogravimetric analysis (TGA) thermogram for the novel crystalline form of (R)-1-{2-[4′-(3-methoxy-propane-1-sulfonyl)-biphenyl-4-yl]-ethyl}-2-methyl-pyrrolidine mono-citrate (FORM 1), which was recorded using a TA Instruments TGA Q500 in a nitrogen atmosphere from 30-250° C., the percent change in weight as a function of temperature was recorded.

FIG. 5 depicts a powder X-ray diffraction (PXRD) pattern for the novel crystalline form of (R)-1-{2-[4′-(3-methoxy-propane-1-sulfonyl)-biphenyl-4-yl]-ethyl}-2-methyl-pyrrolidine di-citrate (from example 1.2), which was recorded using a PANalytical X'Pert Plus Powder X-Ray Diffractometer in the 2θ geometry; scanning angles 5.0°-40.0° 2θ.

FIG. 6 depicts a differential scanning calorimetry (DSC) thermogram for the novel crystalline form of (R)-1-{2-[4′-(3-methoxy-propane-1-sulfonyl)-biphenyl-4-yl]-ethyl}-2-methyl-pyrrolidine di-citrate (from example 1.2) using a TA Instruments DSC Q1000; 25-170° C. at 10° C./min.

FIG. 7 depicts a FTIR spectrum for the novel crystalline form of (R)-1-{2-[4′-(3-methoxy-propane-1-sulfonyl)-biphenyl-4-yl]-ethyl}-2-methyl-pyrrolidine di-citrate (from example 1.2), which was recorded using a Bruker Tensor 27 FTIR spectrometer running OPUS 4.2 software.

FIG. 8 depicts a dynamic vapor sorption (DVS) scan for the novel crystalline form of (R)-1-{2-[4′-(3-methoxy-propane-1-sulfonyl)-biphenyl-4-yl]-ethyl}-2-methyl-pyrrolidine di-citrate (from example 1.2), which was recorded using a VTI-SGA100.

FIG. 9 depicts a thermogravimetric analysis (TGA) thermogram for the novel crystalline form of (R)-1-{2-[4′-(3-methoxy-propane-1-sulfonyl)-biphenyl-4-yl]-ethyl}-2-methyl-pyrrolidine di-citrate (from example 1.2), which was recorded using a TA Instruments TGA Q500 in a nitrogen atmosphere from 30-250° C., the percent change in weight as a function of temperature was recorded.

FIG. 10 depicts a selected region of the FTIR spectrum for the novel crystalline form of (R)-1-{2-[4′-(3-methoxy-propane-1-sulfonyl)-biphenyl-4-yl]-ethyl}-2-methyl-pyrrolidine mono-citrate (FORM 1), which was recorded using a Bruker Tensor 27 FTIR spectrometer running OPUS 4.2 software.

FIG. 11 depicts a selected region of the FTIR spectrum for the novel crystalline form of (R)-1-{2-[4′-(3-methoxy-propane-1-sulfonyl)-biphenyl-4-yl]-ethyl}-2-methyl-pyrrolidine di-citrate (from example 1.2), which was recorded using a Bruker Tensor 27 FTIR spectrometer running OPUS 4.2 software.

FIG. 12 depicts a powder X-ray diffraction (PXRD) pattern for the novel crystalline form of (R)-1-{2-[4′-(3-methoxy-propane-1-sulfonyl)-biphenyl-4-yl]-ethyl}-2-methyl-pyrrolidine maleate, which was recorded using a PANalytical X'Pert PRO MPD Powder X-Ray Diffractometer in the 2θ geometry; scanning angles 5.0°-40.0° 2θ.

FIG. 13 depicts a differential scanning calorimetry (DSC) thermogram for the novel crystalline form of (R)-1-{2-[4′-(3-methoxy-propane-1-sulfonyl)-biphenyl-4-yl]-ethyl}-2-methyl-pyrrolidine maleate, which was recorded using a TA Instruments DSC Q2000; 25-210° C. at 10° C./min.

FIG. 13 depicts a thermogravimetric analysis (TGA) thermogram for the novel crystalline form of (R)-1-{2-[4′-(3-methoxy-propane-1-sulfonyl)-biphenyl-4-yl]-ethyl}-2-methyl-pyrrolidine maleate, which was recorded using a TA Instruments TGA Q500 in a nitrogen atmosphere from 25-250° C., the percent change in weight as a function of temperature was recorded.

FIG. 14 depicts a dynamic vapor sorption (DVS) scan for the novel crystalline form of (R)-1-{2-[4′-(3-methoxy-propane-1-sulfonyl)-biphenyl-4-yl]-ethyl}-2-methyl-pyrrolidine maleate, which was recorded using a VTI-SGA100.

FIG. 15 depicts a Raman spectrum for the novel crystalline form of (R)-1-{2-[4′-(3-methoxy-propane-1-sulfonyl)-biphenyl-4-yl]-ethyl}-2-methyl-pyrrolidine maleate, which was recorded using a Thermo Nicolet NXR6700 FT-Raman instrument.

FIG. 16 depicts a powder X-ray diffraction (PXRD) pattern for the novel crystalline form of (R)-1-{2-[4′-(3-methoxy-propane-1-sulfonyl)-biphenyl-4-yl]-ethyl}-2-methyl-pyrrolidine di-citrate (from example 1.5), which was recorded using a PANalytical X'Pert PRO MPD Powder X-Ray Diffractometer in the 2θ geometry; scanning angles 5.0°-40.0° 2θ.

FIG. 17 depicts a differential scanning calorimetry (DSC) thermogram for the novel crystalline form of (R)-1-{2-[4′-(3-methoxy-propane-1-sulfonyl)-biphenyl-4-yl]-ethyl}-2-methyl-pyrrolidine di-citrate (from example 1.5), which was recorded using a TA Instruments DSC Q1000; 25-200° C. at 10° C./min.

FIG. 18 depicts a thermogravimetric analysis (TGA) thermogram for the novel crystalline form of (R)-1-{2-[4′-(3-methoxy-propane-1-sulfonyl)-biphenyl-4-yl]-ethyl}-2-methyl-pyrrolidine di-citrate (from example 1.5), which was recorded using a TA Instruments TGA Q500 in a nitrogen atmosphere from 25-200° C., the percent change in weight as a function of temperature was recorded.

FIG. 19 depicts a Raman spectrum for the novel crystalline form of (R)-1-{2-[4′-(3-methoxy-propane-1-sulfonyl)-biphenyl-4-yl]-ethyl}-2-methyl-pyrrolidine di-citrate (from example 1.5), which was recorded using a Thermo Nicolet NXR6700 FT-Raman instrument.

FIG. 20 depicts a powder X-ray diffraction (PXRD) pattern for the novel crystalline form of (R)-1-{2-[4′-(3-methoxy-propane-1-sulfonyl)-biphenyl-4-yl]-ethyl}-2-methyl-pyrrolidine mono-citrate (FORM 2), which was recorded using a PANalytical X'Pert PRO MPD Powder X-Ray Diffractometer in the 2θ geometry; scanning angles 5.0°-40.0° 2θ.

FIG. 21 depicts a differential scanning calorimetry (DSC) thermogram for the novel crystalline form of (R)-1-{2-[4′-(3-methoxy-propane-1-sulfonyl)-biphenyl-4-yl]-ethyl}-2-methyl-pyrrolidine mono-citrate (FORM 2), which was recorded using a TA Instruments DSC Q2000; 25-210° C. at 10° C./min.

FIG. 22 depicts a Raman spectrum for the novel crystalline form of (R)-1-{2-[4′-(3-methoxy-propane-1-sulfonyl)-biphenyl-4-yl]-ethyl}-2-methyl-pyrrolidine mono-citrate (FORM2), which was recorded using a Thermo Nicolet NXR6700 FT-Raman instrument.

FIG. 23 depicts a powder X-ray diffraction (PXRD) pattern for the novel crystalline form of (R)-1-{2-[4′-(3-methoxy-propane-1-sulfonyl)-biphenyl-4-yl]-ethyl}-2-methyl-pyrrolidine hydrochloride, which was recorded using a PANalytical X'Pert PRO MPD Powder X-Ray Diffractometer in the 2θ geometry; scanning angles 5.0°-40.0° 2θ.

FIG. 24 depicts a differential scanning calorimetry (DSC) thermogram for the novel crystalline form of (R)-1-{2-[4′-(3-methoxy-propane-1-sulfonyl)-biphenyl-4-yl]-ethyl}-2-methyl-pyrrolidine hydrochloride, which was recorded using a TA Instruments DSC Q2000; 25-250° C. at 10° C./min.

FIG. 25 depicts a thermogravimetric analysis (TGA) thermogram for the novel crystalline form of (R)-1-{2-[4′-(3-methoxy-propane-1-sulfonyl)-biphenyl-4-yl]-ethyl}-2-methyl-pyrrolidine hydrochloride, which was recorded using a TA Instruments TGA Q500 in a nitrogen atmosphere from 25-250° C., the percent change in weight as a function of temperature was recorded.

FIG. 26 depicts a Raman spectrum for the novel crystalline form of (R)-1-{2-[4′-(3-methoxy-propane-1-sulfonyl)-biphenyl-4-yl]-ethyl}-2-methyl-pyrrolidine hydrochloride, which was recorded using a Thermo Nicolet NXR6700 FT-Raman instrument.

FIG. 27 depicts a selected region of the Raman spectrum for the novel crystalline form of (R)-1-{2-[4′-(3-methoxy-propane-1-sulfonyl)-biphenyl-4-yl]-ethyl}-2-methyl-pyrrolidine hydrochloride, which was recorded using a Thermo Nicolet NXR6700 FT-Raman instrument.

FIG. 28 depicts a differential scanning calorimetry (DSC) thermogram for the novel crystalline form of (R)-1-{2-[4′-(3-methoxy-propane-1-sulfonyl)-biphenyl-4-yl]-ethyl}-2-methyl-pyrrolidine di-citrate (from example 1.7, method 4) using a TA Instruments DSC Q1000; 25-260° C. at 10° C./min.

### DETAILED DESCRIPTION

The present invention is directed to, inter alia, salts of (R)-1-{2-[4′-(3-methoxy-propane-1-sulfonyl)-biphenyl-4-yl]-ethyl}-2-methyl-pyrrolidine and crystalline forms thereof.

One aspect of the present invention is directed to (R)-1-{2-[4′-(3-methoxy-propane-1-sulfonyl)-biphenyl-4-yl]-ethyl}-2-methyl-pyrrolidine mono-citrate.

Another aspect of the present invention is directed to crystalline forms of (R)-1-{2-[4′-(3-methoxy-propane-1-sulfonyl)-biphenyl-4-yl]-ethyl}-2-methyl-pyrrolidine mono-citrate.

Another aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate.

One aspect of the present invention is directed to (R)-1-{2-[4′-(3-methoxy-propane-1-sulfonyl)-biphenyl-4-yl]-ethyl}-2-methyl-pyrrolidine di-citrate.

Another aspect of the present invention is directed to a crystalline form of (R)-1-{2-[4′-(3-methoxy-propane-1-sulfonyl)-biphenyl-4-yl]-ethyl}-2-methyl-pyrrolidine di-citrate.

One aspect of the present invention is directed to a maleate salt of (R)-1-{2-[4′-(3-methoxy-propane-1-sulfonyl)-biphenyl-4-yl]-ethyl}-2-methyl-pyrrolidine and crystalline forms thereof.

One aspect of the present invention is directed to (R)-1-{2-[4′-(3-methoxy-propane-1-sulfonyl)-biphenyl-4-yl]-ethyl}-2-methyl-pyrrolidine maleate.

Another aspect of the present invention is directed to a crystalline form of (R)-1-{2-[4′-(3-methoxy-propane-1-sulfonyl)-biphenyl-4-yl]-ethyl}-2-methyl-pyrrolidine maleate.

One aspect of the present invention is directed to a hydrochloride salt of (R)-1-{2-[4′-(3-methoxy-propane-1-sulfonyl)-biphenyl-4-yl]-ethyl}-2-methyl-pyrrolidine and crystalline forms thereof.

One aspect of the present invention is directed to (R)-1-{2-[4′-(3-methoxy-propane-1-sulfonyl)-biphenyl-4-yl]-ethyl}-2-methyl-pyrrolidine hydrochloride.

Another aspect of the present invention is directed to a crystalline form of (R)-1-{2-[4′-(3-methoxy-propane-1-sulfonyl)-biphenyl-4-yl]-ethyl}-2-methyl-pyrrolidine hydrochloride.

The crystalline forms of (R)-1-{2-[4′-(3-methoxy-propane-1-sulfonyl)-biphenyl-4-yl]-ethyl}-2-methyl-pyrrolidine mono-citrate, (R)-1-{2-[4′-(3-methoxy-propane-1-sulfonyl)-biphenyl-4-yl]-ethyl}-2-methyl-pyrrolidine di-citrate, (R)-1-{2-[4′-(3-methoxy-propane-1-sulfonyl)-biphenyl-4-yl]-ethyl}-2-methyl-pyrrolidine maleate and (R)-1-{2-[4′-(3-methoxy-propane-1-sulfonyl)-biphenyl-4-yl]-ethyl}-2-methyl-pyrrolidine hydrochloride can be identified by their unique solid state signatures with respect to, for example, differential scanning calorimetry (DSC), powder X-ray diffraction (PXRD), and other solid state methods. Further characterization with respect to water or solvent content of the crystalline forms can be gauged by any of the following methods for example, thermogravimetric analysis (TGA), DSC and the like. For DSC, it is known that the temperatures observed for thermal events will depend upon the rate of temperature change as well as sample preparation technique and the particular instrument employed. Thus, the values reported herein relating to DSC thermograms can vary by plus or minus about 4° C. For PXRD, the relative intensities of the peaks can vary, depending upon the sample preparation technique, the sample mounting procedure and the particular instrument employed. Moreover, instrument variation and other factors can often affect the 2θ values. Therefore, the peak assignments of diffraction patterns can vary by plus or minus about 0.2°. The physical properties of the crystalline forms of (R)-1-{2-[4′-(3-methoxy-propane-1-sulfonyl)-biphenyl-4-yl]-ethyl}-2-methyl-pyrrolidine mono-citrate, (R)-1-{2-[4′-(3-methoxy-propane-1-sulfonyl)-biphenyl-4-yl]-ethyl}-2-methyl-pyrrolidine di-citrate, (R)-1-{2-[4′-(3-methoxy-propane-1-sulfonyl)-biphenyl-4-yl]-ethyl}-2-methyl-pyrrolidine maleate and (R)-1-{2-[4′-(3-methoxy-propane-1-sulfonyl)-biphenyl-4-yl]-ethyl}-2-methyl-pyrrolidine hydrochloride of the present invention are summarized in Table 1 below.

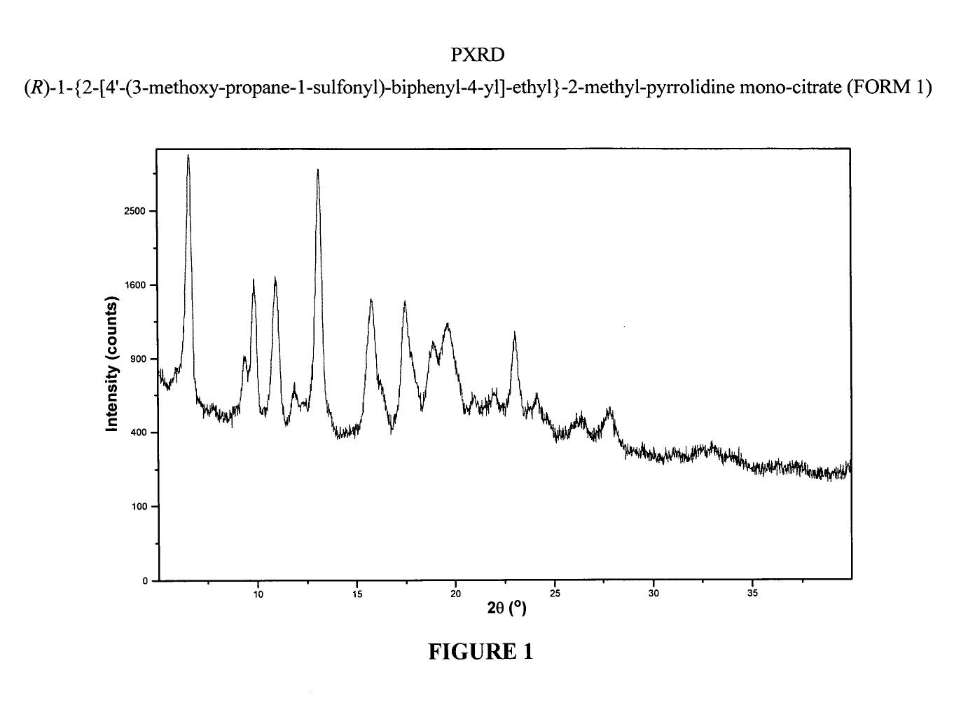

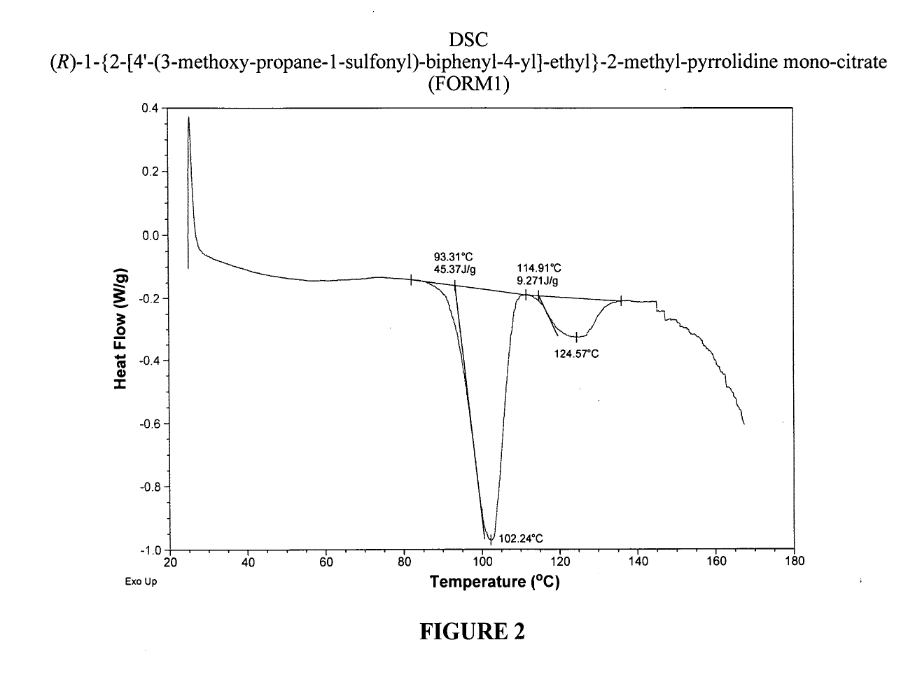

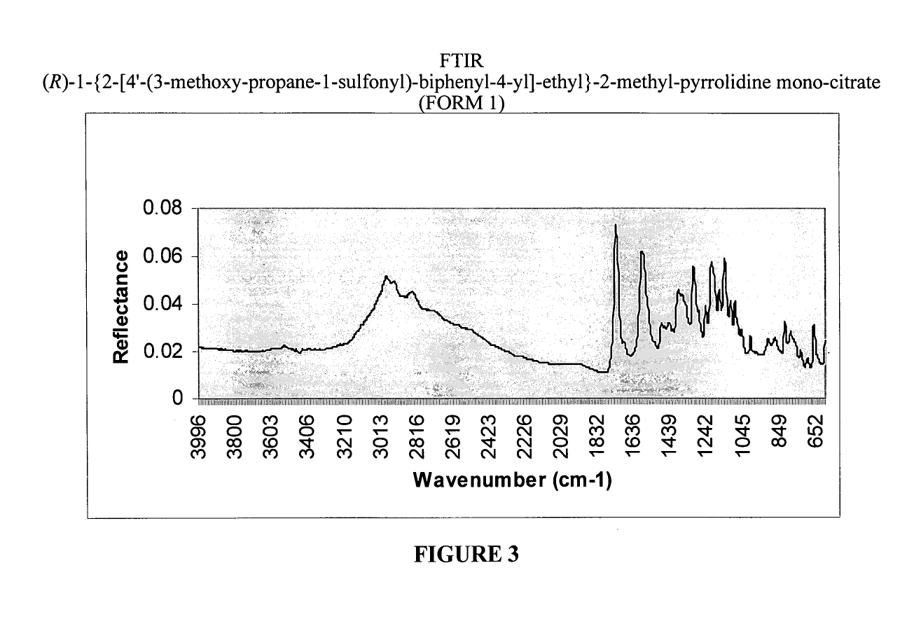

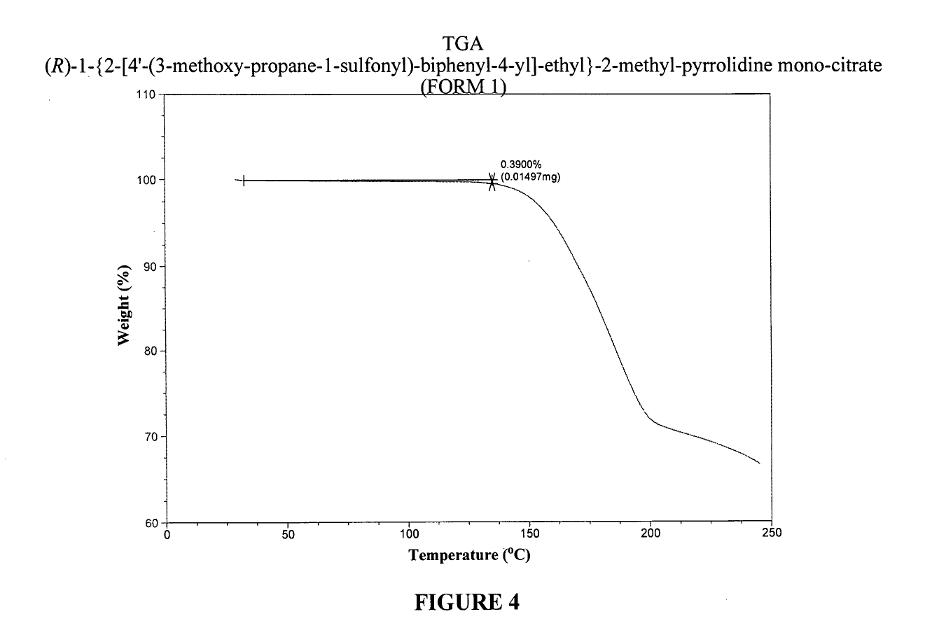

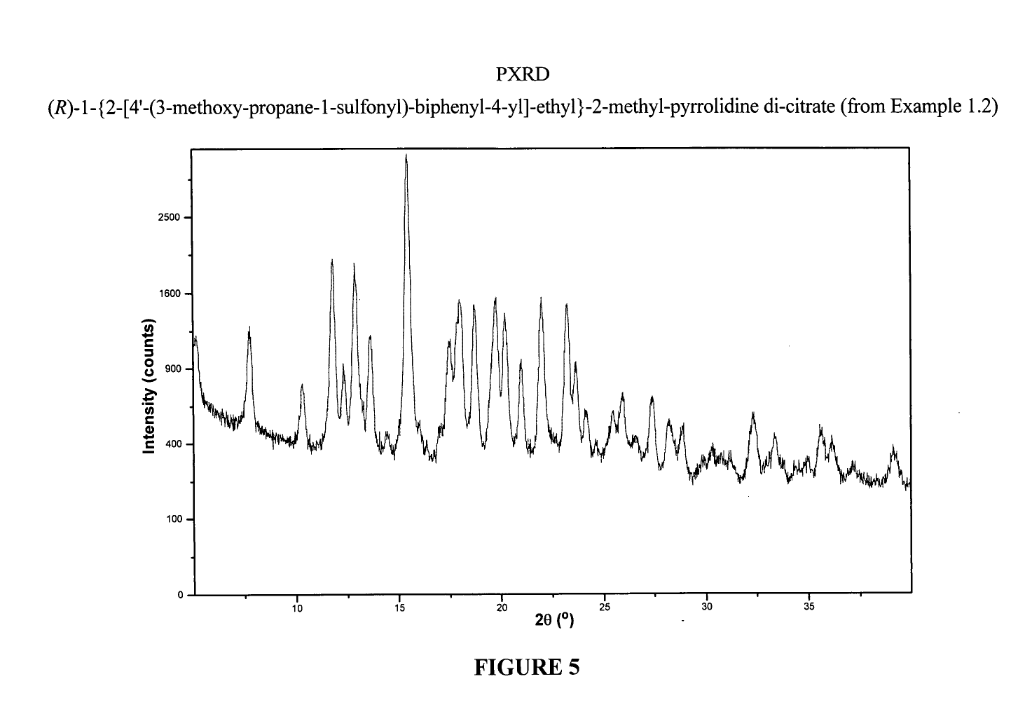

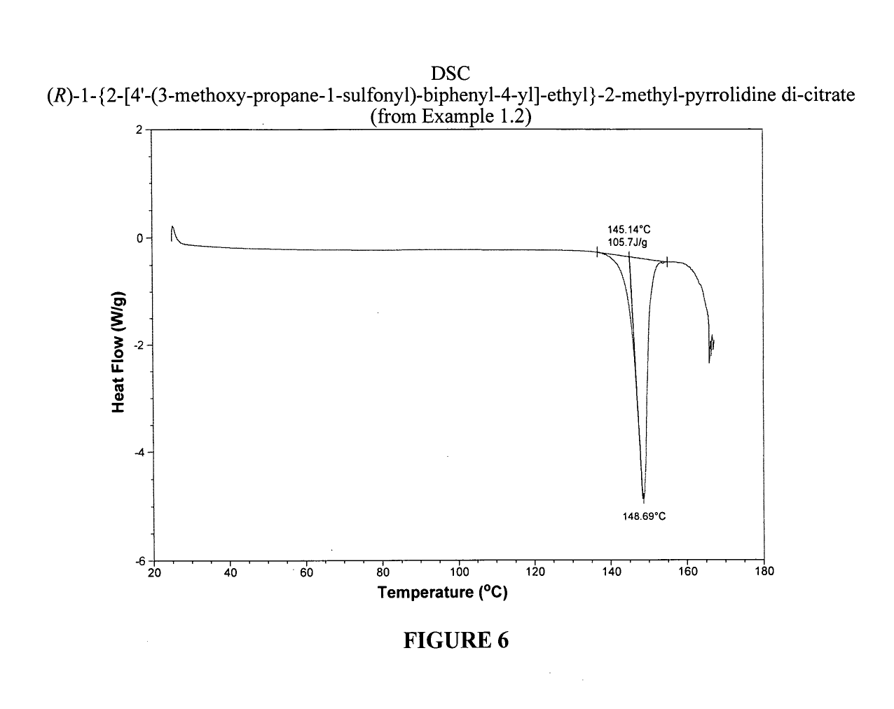

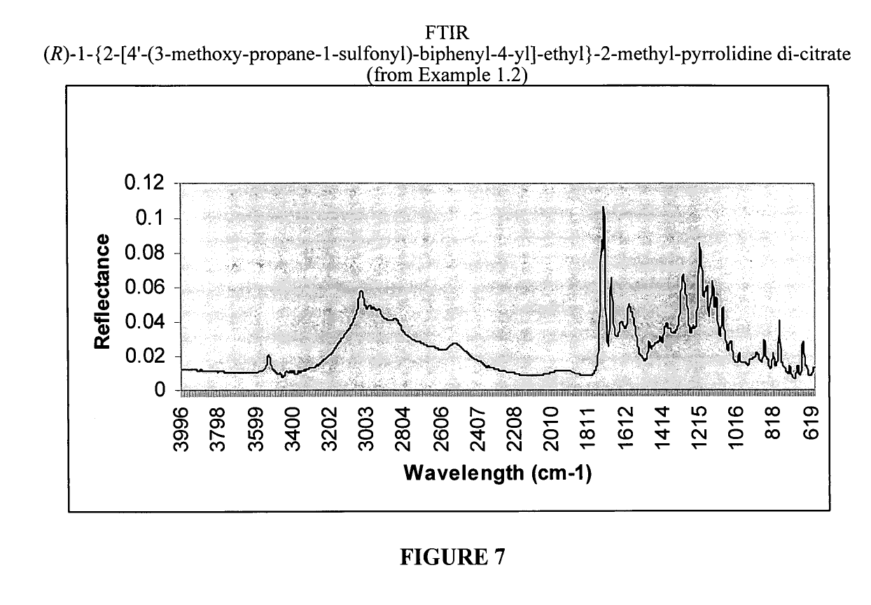

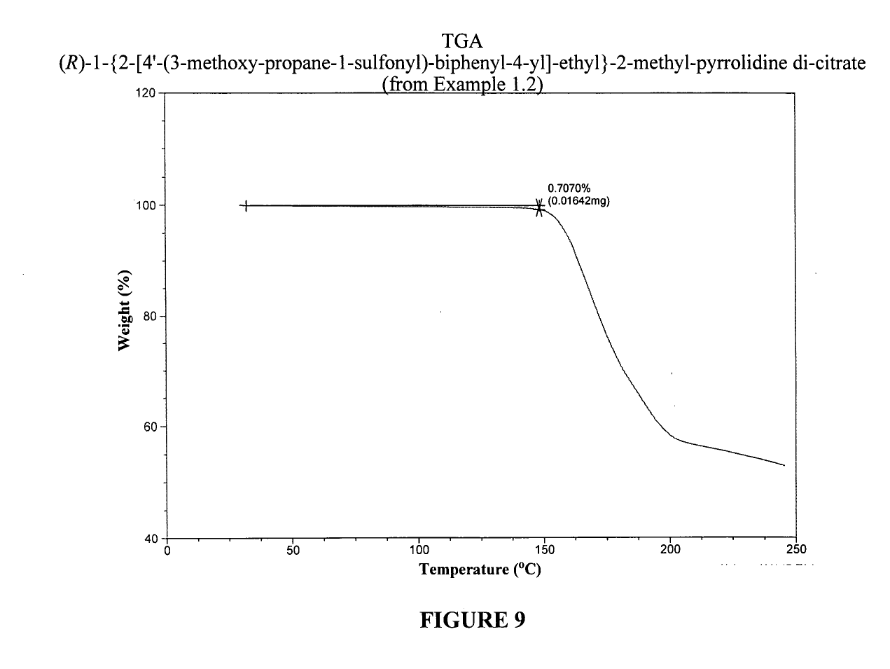

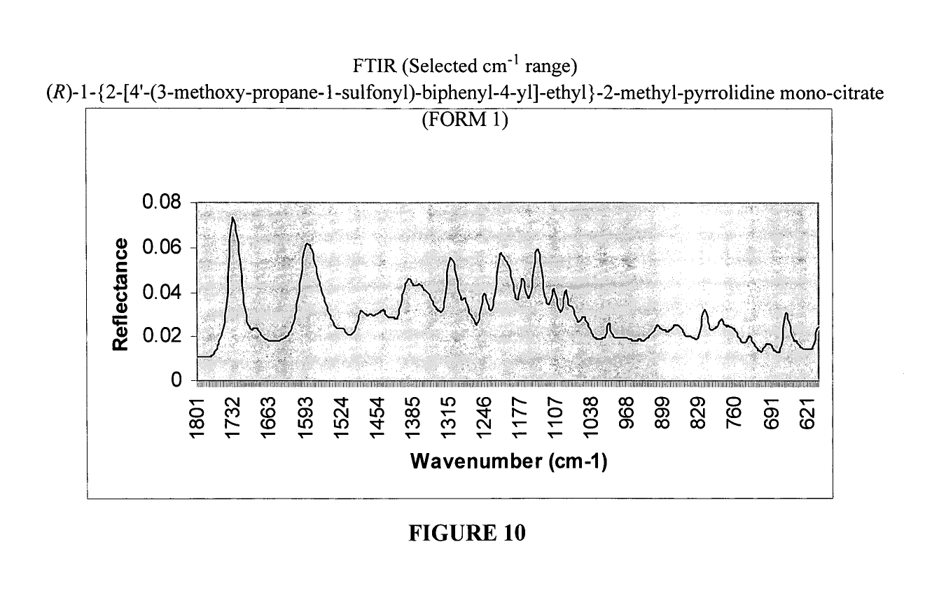

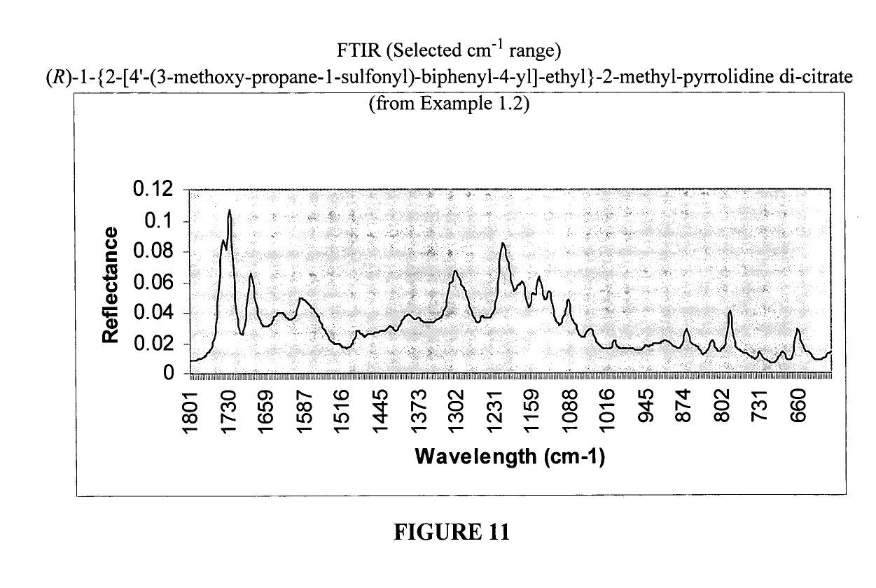

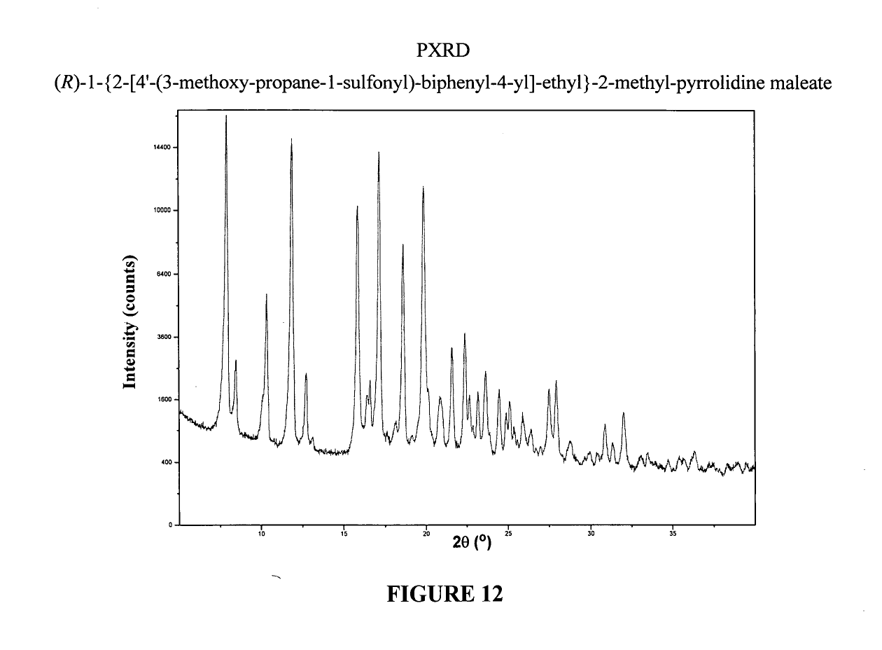

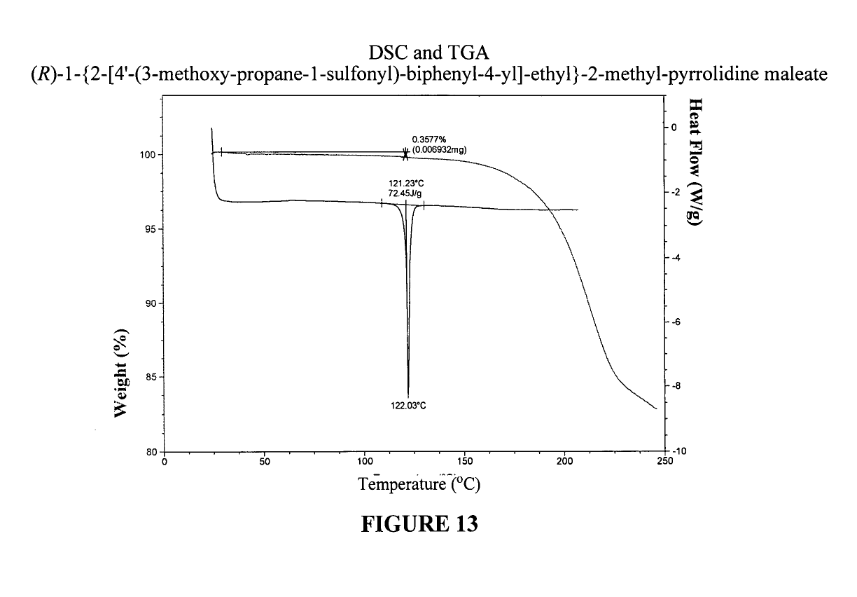

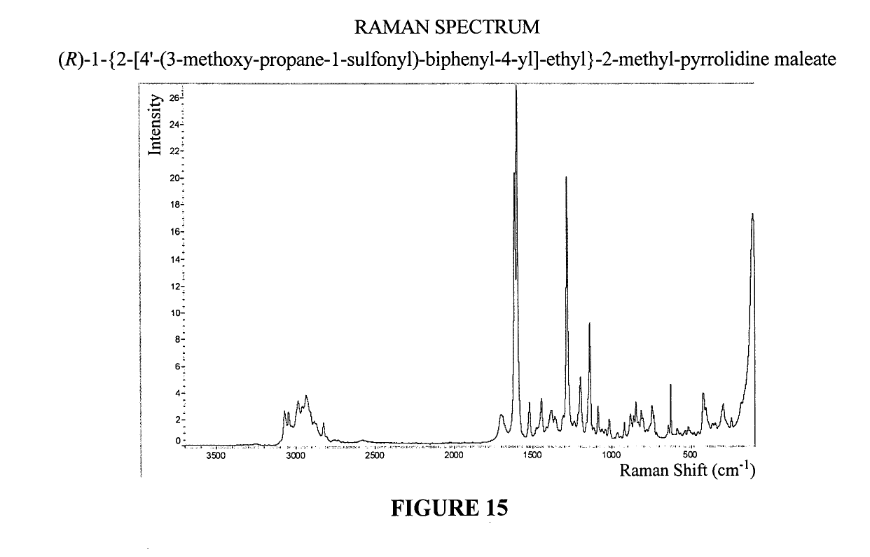

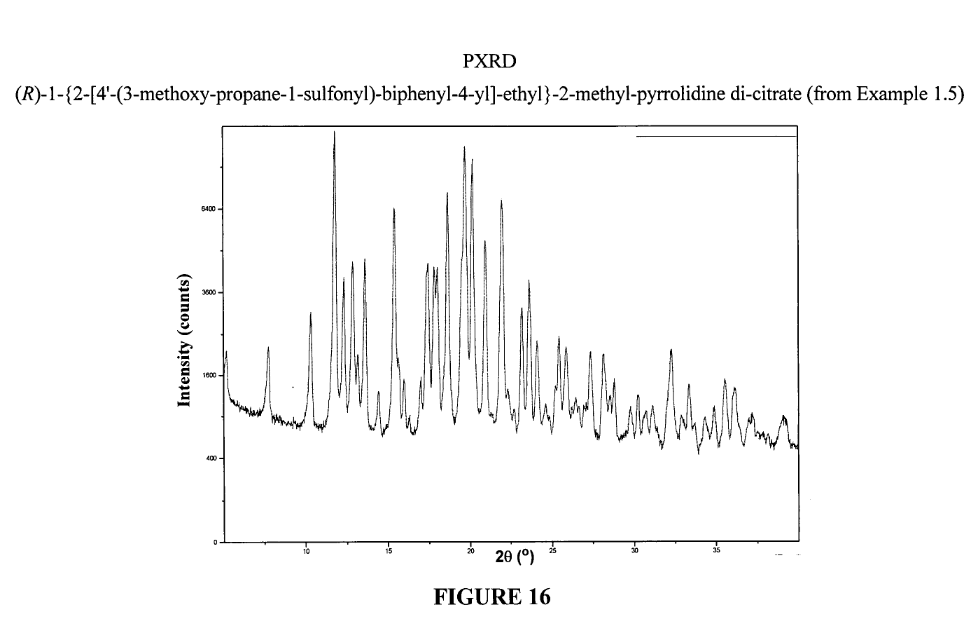

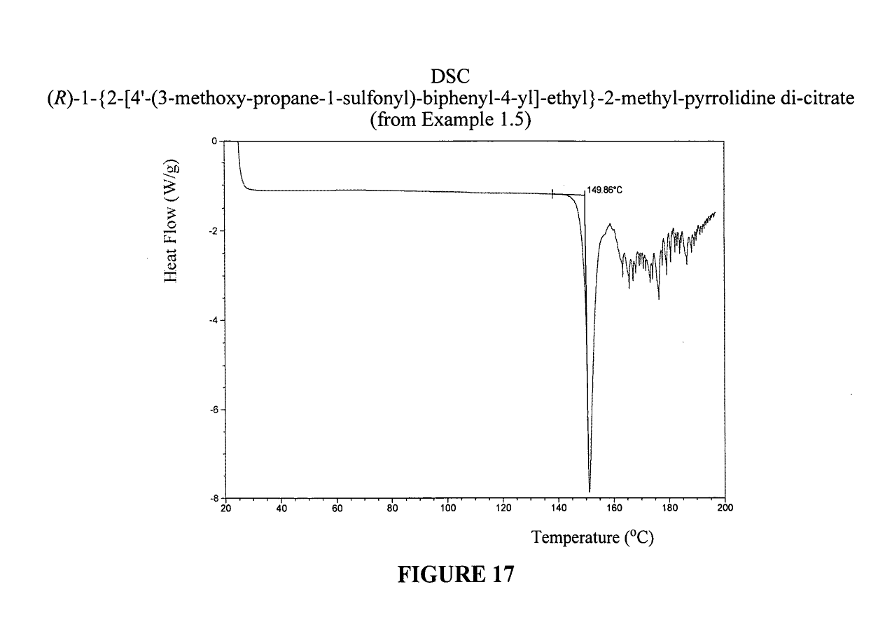

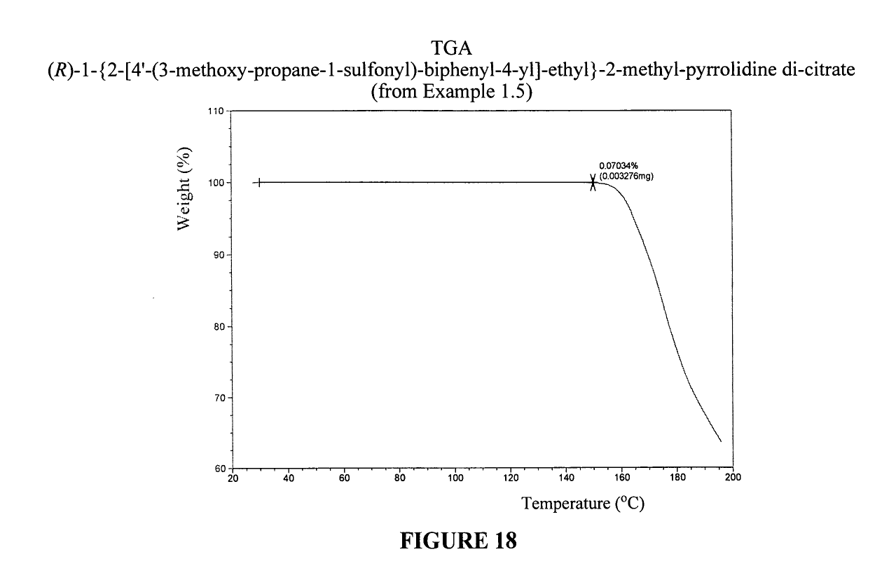

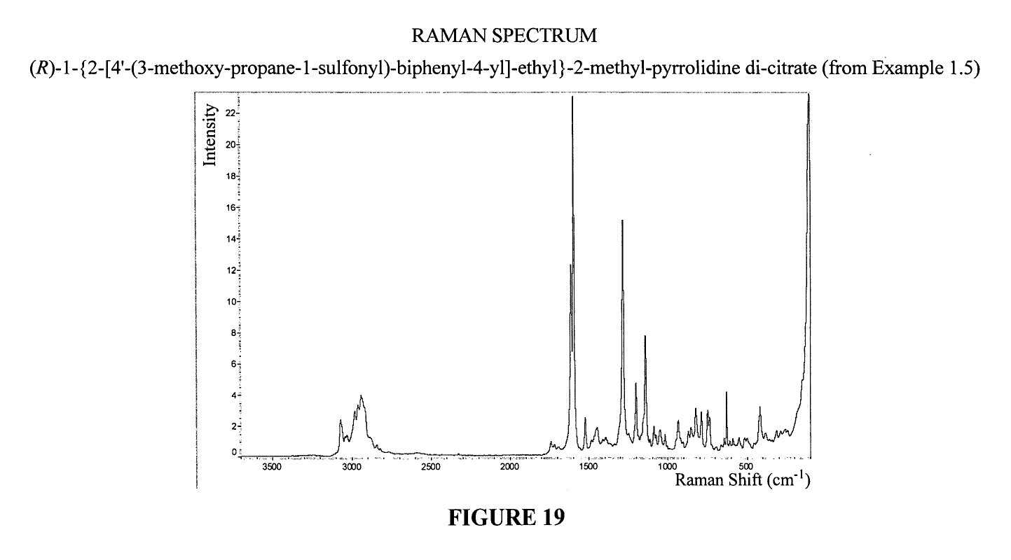

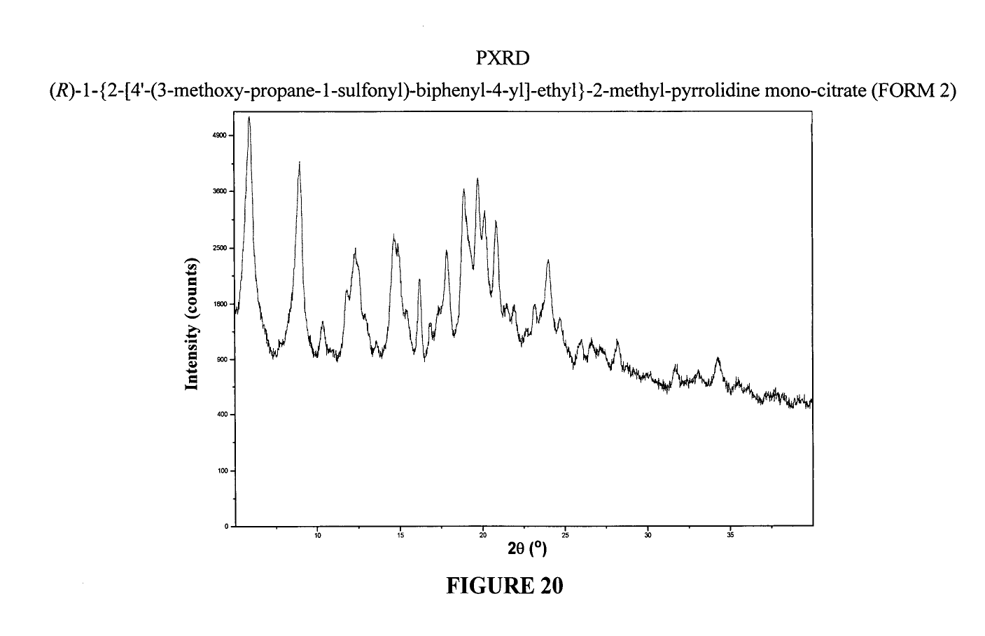

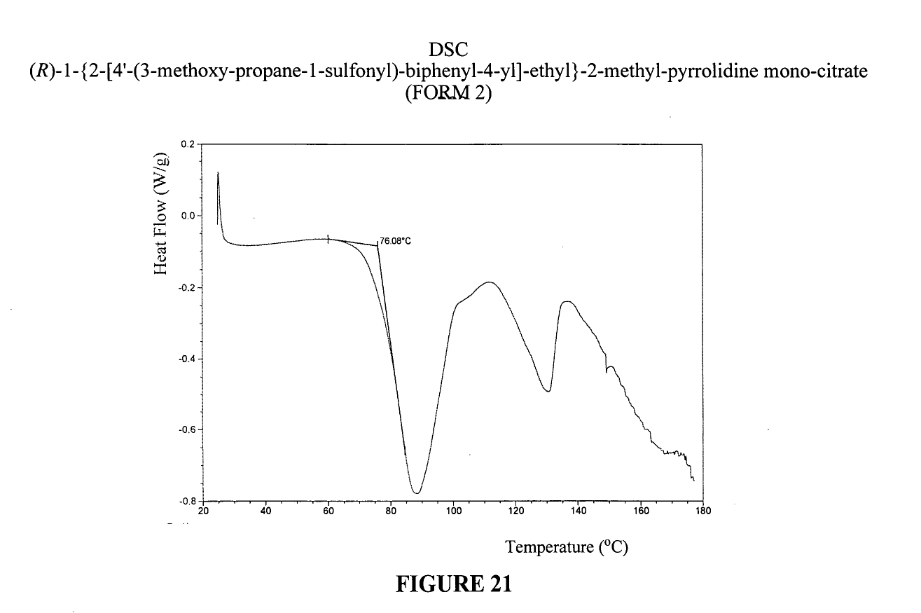

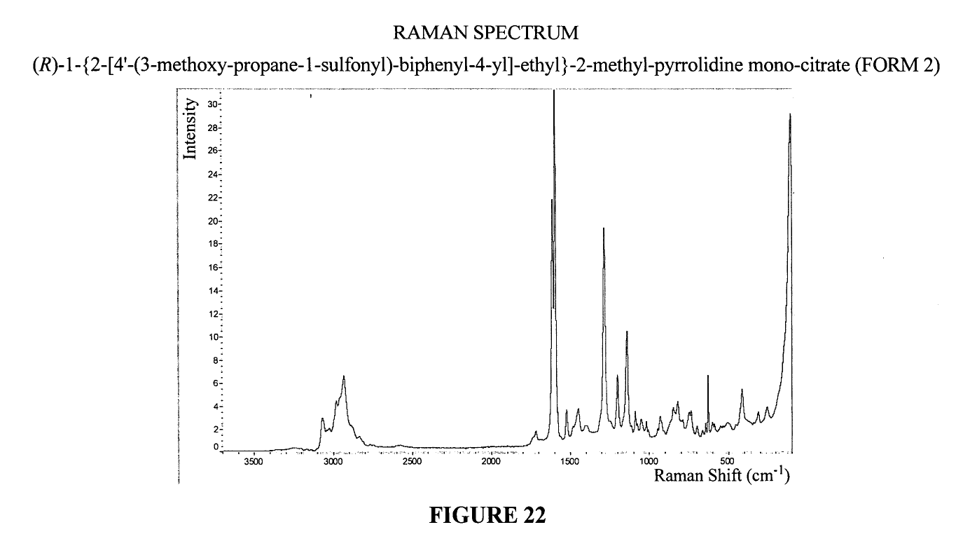

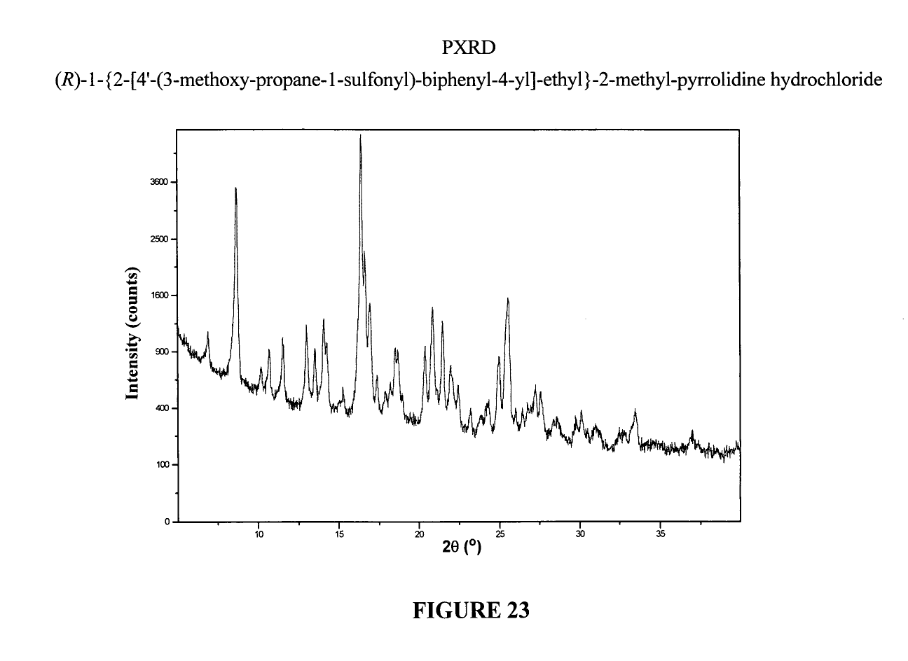

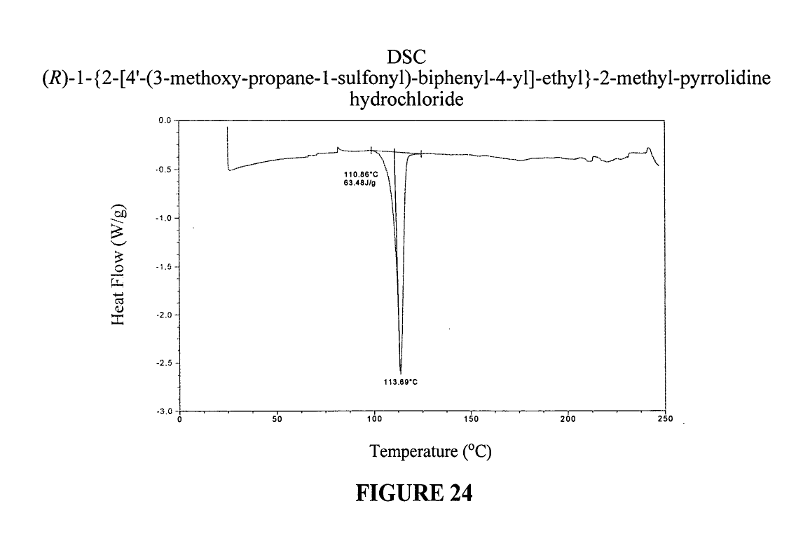

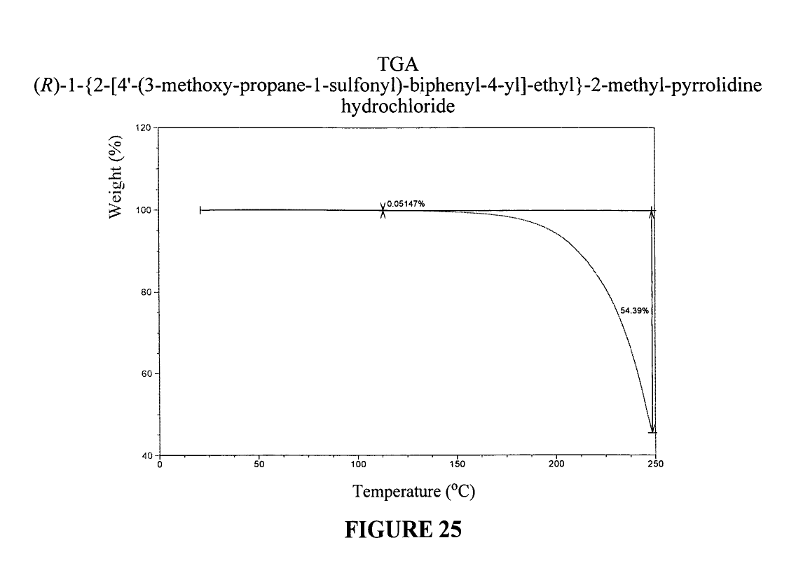

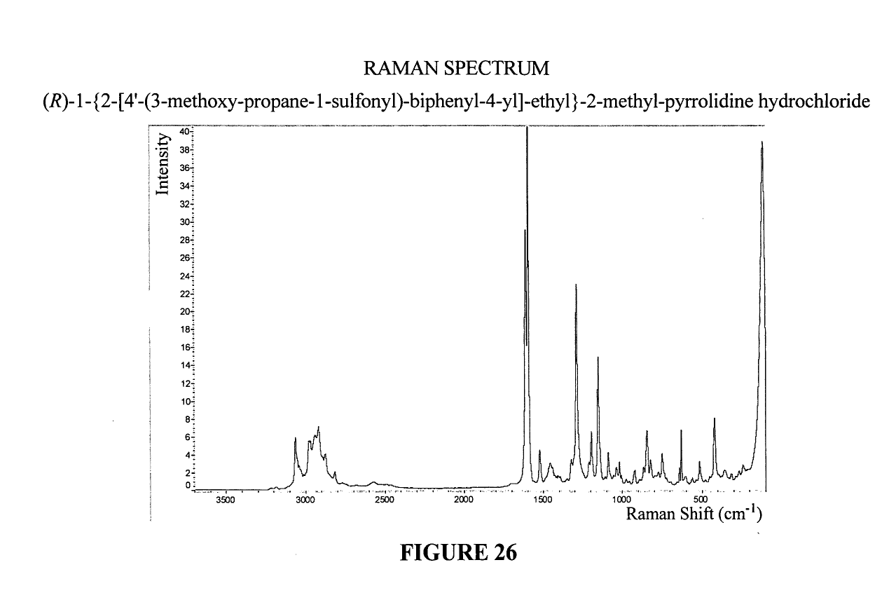

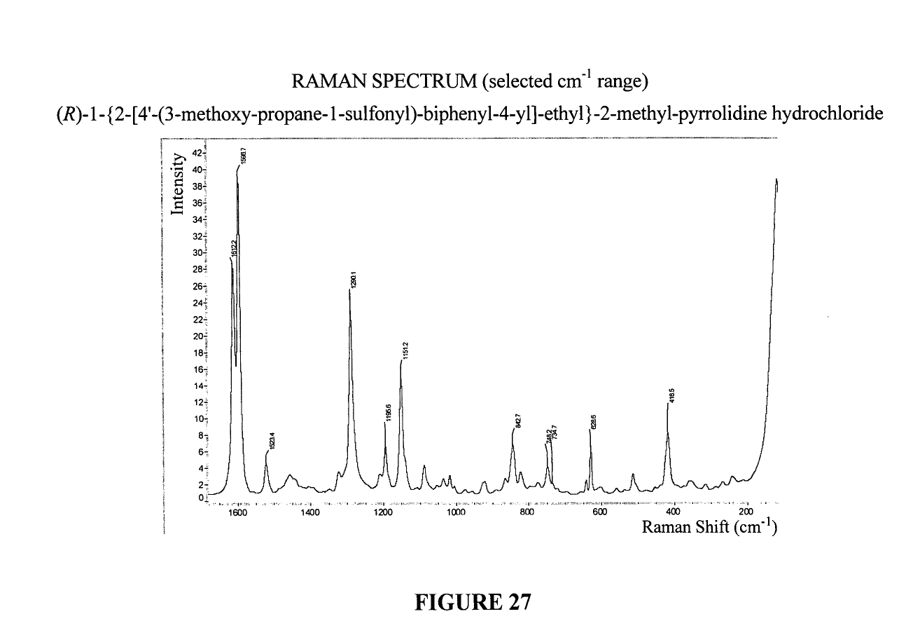

TABLE 1

| | Crystalline form of (R)-1-{2-[4′-(3- | Crystalline form of (R)-1-{2-[4′-(3- |
|---|---|---|
| | methoxy-propane-1-sulfonyl)- | methoxy-propane-1-sulfonyl)- |
| | biphenyl-4-yl]-ethyl}-2-methyl- | biphenyl-4-yl]-ethyl}-2-methyl- |
| | pyrrolidine mono-citrate (FORM 1) | pyrrolidine mono-citrate (FORM 2) |
| PXRD | FIG. 1: Peaks of ≧ 16% relative | FIG. 20: Peaks of ≧ 20% relative |
| | intensity at 6.6°, 9.9°, 11.0°, 13.1°, | intensity at 5.9°, 6.0°, 9.0°, 11.8°, |
| | 15.8°, 17.5°, 18.9°, 19.6° and 23.0° 2θ | 12.3°, 12.6°, 14.7°, 15.0°, 16.2°, |
| | | 17.9°, 18.9°, 19.8°, 20.2°, 20.8°, |
| | | 20.9°, 21.5°, 21.9°, 23.2°, 24.0° and |
| | | 24.7° 2θ |
| DSC | FIG. 2: endotherms at about 102° C. | FIG. 21: endotherm with an |
| | and about 125° C. | extrapolated onset temperature at |
| | | about 76° C. and a peak temperature at |
| | | about 88° C. |
| FTIR or | FIG. 3 (FTIR): Peaks at 2980 2930, | FIG. 22 (RAMAN): Peaks at about |
| RAMAN | 2830, 1732, 1587, 1312, 1213, and | 413 cm⁻¹, about 1140 cm⁻¹, about 1284 |
| | 1142 cm⁻¹ | cm⁻¹, about 1596 cm⁻¹, about 1612 |
| | | cm⁻¹, about 2935 cm⁻¹ and about 3072 |
| | | cm⁻¹ |
| TGA | FIG. 4: negligible weight loss below | |
| | about 125° C. | |
| | Crystalline form of (R)-1-{2-[4′-(3- | Crystalline form of (R)-1-{2-[4′-(3- |
| | methoxy-propane-1-sulfonyl)- | methoxy-propane-1-sulfonyl)- |
| | biphenyl-4-yl]-ethyl}-2-methyl- | biphenyl-4-yl]-ethyl}-2-methyl- |
| | pyrrolidine di-citrate | pyrrolidine di-citrate |
| | (from Example 1.2) | (from Example 1.5) |
| PXRD | FIG. 5: Peaks of ≧ 12% relative | FIG. 16: Peaks of ≧ 20% relative |
| | intensity at 7.8°, 10.3°, 11.8°, 12.9°, | intensity at 7.7°, 10.3°, 11.8°, 12.9°, |
| | 13.6°, 15.5°, 18.1°, 18.7°, 19.7°, 20.2°, | 13.6°, 15.4°, 17.4°, 17.5°, 17.8°, |
| | 22.0°, and 23.2° 2θ | 18.0°, 18.7°, 19.5°, 19.7°, 20.2°, |
| | | 20.9°, 21.3°, 22.0°, 22.3°, 23.2° and |
| | | 25.4° 2θ |
| DSC | FIG. 6: an endotherm at about 149° C. | FIG. 17: endotherm with an |
| | | extrapolated onset temperature at |
| | | about 150° C. and a peak temperature |
| | | at about 151° C. |
| FTIR or | FIG. 7 (FTIR): Peaks at 3528, 3030, | FIG. 19 (RAMAN): Peaks at about |
| RAMAN | 2853, 1738, 1726, 1686, 1593, 1304, | 416 cm⁻¹, about 746 cm⁻¹, about 788 |
| | 1213, 1146 cm⁻¹ | cm⁻¹, about 1284 cm⁻¹, about 1596 |
| | | cm⁻¹, about 1612 cm⁻¹, about 2963 |
| | | cm⁻¹ and about 3073 cm⁻¹ |
| TGA | FIG. 9: negligible weight loss below | FIG. 18: negligible weight loss |
| | about 150° C. | below about 150° C. |
| | Crystalline form of (R)-1-{2-[4′-(3- | Crystalline form of (R)-1-{2-[4′-(3- |
| | methoxy-propane-1-sulfonyl)- | methoxy-propane-1-sulfonyl)- |
| | biphenyl-4-yl]-ethyl}-2-methyl- | biphenyl-4-yl]-ethyl}-2-methyl- |
| | pyrrolidine maleate | pyrrolidine hydrochloride |
| PXRD | FIG. 12: Peaks of ≧ 12% relative | FIG. 23: Peaks of ≧ 12% relative |
| | intensity at 7.9°, 8.5°, 10.3°, 11.9°, | intensity at 8.7°, 11.6°, 13.0°, 13.5°, |
| | 12.7°, 15.9°, 17.2°, 18.6°, 19.9°, 20.2°, | 14.1°, 14.3°, 16.2°, 16.4°, 16.7°, |
| | 21.6°, 22.4°, and 23.6° 2θ | 17.0°, 18.5°, 18.7°, 20.4°, 20.9°, |
| | | 21.5°, 22.0°, 25.0°, 25.4°, and 25.6° |
| | | 2θ |
| DSC | FIG. 13: an endotherm with an | FIG. 24: an endotherm with an |
| | extrapolated onset temperature at about | extrapolated onset temperature at |
| | 121° C., a peak temperature at about | about 111° C., a peak temperature at |
| | 122° C. and an associated heat flow of | about 114° C. and an associated heat |
| | 72 joules per gram | flow of 63 joules per gram |
| RAMAN | FIG. 15: Peaks at about 423 cm⁻¹, | FIG. 26: Peaks at about 417 cm⁻¹, |
| | about 883 cm⁻¹, about 1017 cm⁻¹, about | about 629 cm⁻¹, about 748 cm⁻¹, about |
| | 1089 cm⁻¹, about 1283 cm⁻¹, about | 843 cm⁻¹, about 1090, about 1151 |
| | 1598 cm⁻¹, about 1613 cm⁻¹, about | cm⁻¹, about 1196 cm⁻¹, about 1290 |
| | 2985 cm⁻¹, about 3045 cm⁻¹ and about | cm⁻¹, about 1523 cm⁻¹, about 1599 |
| | 3072 cm⁻¹ | cm⁻¹, about 1612 cm⁻¹, about 2817 |
| | | cm⁻¹, about 2920 cm⁻¹and about 3066 |
| | | cm⁻¹ |
| TGA | FIG. 13: negligible weight loss below | FIG. 25: negligible weight loss |
| | about 121° C. | below about 111° C. |

The negligible weight loss observed in the TGA data suggests that (R)-1-{2-[4′-(3-methoxy-propane-1-sulfonyl)-biphenyl-4-yl]-ethyl}-2-methyl-pyrrolidine mono-citrate and (R)-1-{2-[4′-(3-methoxy-propane-1-sulfonyl)-biphenyl-4-yl]-ethyl}-2-methyl-pyrrolidine di-citrate are anhydrous, non-solvated crystalline forms.

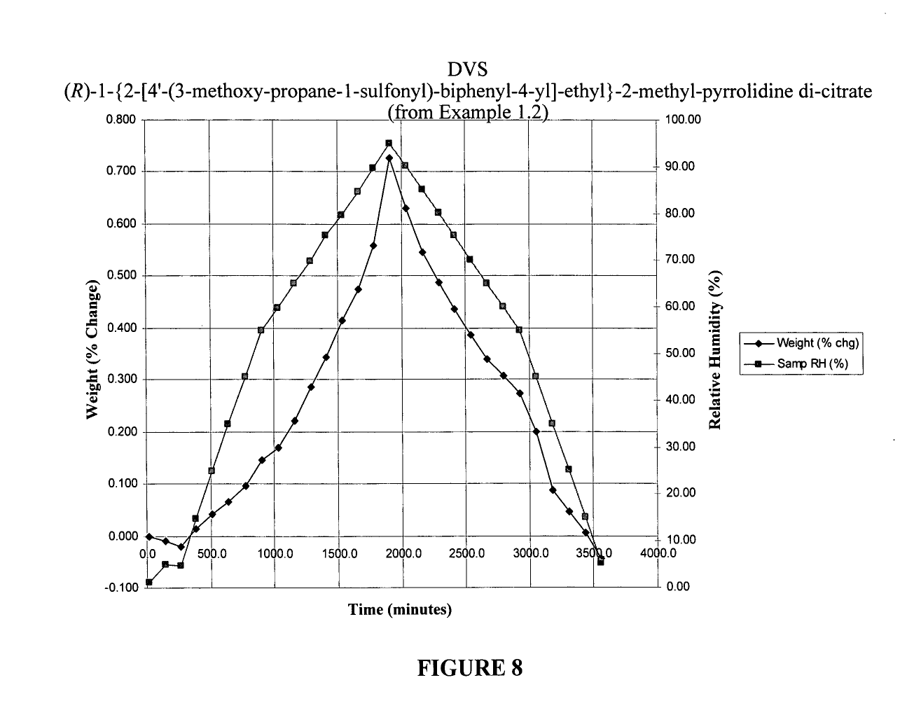

In addition, the DVS data (see FIG. 8) for the crystalline form of (R)-1-{2-[4′-(3-methoxy-propane-1-sulfonyl)-biphenyl-4-yl]-ethyl}-2-methyl-pyrrolidine di-citrate reveals that it is non-hygroscopic.

The negligible weight loss observed in the TGA data (see FIG. 13) suggests that (R)-1-{2-[4′-(3-methoxy-propane-1-sulfonyl)-biphenyl-4-yl]-ethyl}-2-methyl-pyrrolidine maleate is an anhydrous, non-solvated crystalline form.

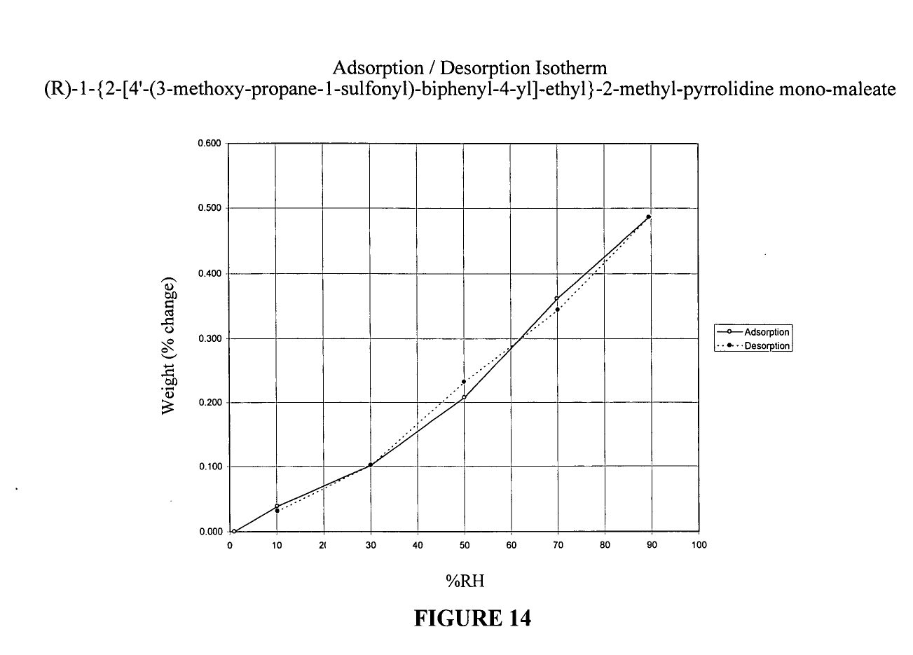

In addition, the DVS data (see FIG. 14) for the crystalline form of (R)-1-{2-[4′-(3-methoxy-propane-1-sulfonyl)-biphenyl-4-yl]-ethyl}-2-methyl-pyrrolidine maleate reveals that it is non-hygroscopic.

The negligible weight loss observed in the TGA data (see FIG. 25) suggests that (R)-1-{2-[4′-(3-methoxy-propane-1-sulfonyl)-biphenyl-4-yl]-ethyl}-2-methyl-pyrrolidine hydrochloride is an anhydrous, non-solvated crystalline form.

Powder X-ray diffraction peaks for 2 forms of (R)-1-{2-[4′-(3-methoxy-propane-1-sulfonyl)-biphenyl-4-yl]-ethyl}-2-methyl-pyrrolidine mono-citrate are shown in Tables 2 and 3 below.

TABLE 2
PXRD Diffraction peaks for (R)-1-{2-[4′-(3-methoxy-propane-1-sulfonyl)-
biphenyl-4-yl]-ethyl}-2-methyl-pyrrolidine mono-citrate (degrees 2θ)
(FORM 1)

| | | | | |
|---|---|---|---|---|
| 6.6 | 7.8 | 9.4 | 9.9 | 11.0 |
| 11.9 | 13.1 | 15.8 | 17.5 | 18.9 |
| 19.6 | 20.9 | 22.0 | 23.0 | 24.1 |
| 26.5 | 27.8 | 33.1 | 37.4 | |

TABLE 3
PXRD Diffraction peaks for (R)-1-{2-[4′-(3-methoxy-propane-1-sulfonyl)-
biphenyl-4-yl]-ethyl}-2-methyl-pyrrolidine mono-citrate (degrees 2θ)
(FORM 2)

| | | | | |
|---|---|---|---|---|
| 5.9 | 6.0 | 9.0 | 10.3 | 11.8 |
| 12.3 | 12.6 | 12.9 | 13.6 | 14.7 |
| 15.0 | 15.4 | 16.2 | 16.8 | 17.3 |
| 17.9 | 18.9 | 19.8 | 20.1 | 20.9 |
| 21.5 | 21.9 | 22.7 | 23.2 | 24.0 |
| 24.7 | 26.0 | 26.7 | 27.2 | 28.2 |
| 34.3 | | | | |

Powder X-ray diffraction peaks for (R)-1-{2-[4′-(3-methoxy-propane-1-sulfonyl)-biphenyl-4-yl]-ethyl}-2-methyl-pyrrolidine di-citrate are shown in Tables 4 and 5 below.

TABLE 4
PXRD Diffraction peaks for (R)-1-{2-[4′-(3-methoxy-
propane-1-sulfonyl)-biphenyl-4-yl]-ethyl}-2-methyl-
pyrrolidine di-citrate (degrees 2θ) (from Example 1.2)

| | | | | |
|---|---|---|---|---|
| 5.2 | 7.8 | 10.3 | 11.8 | 12.3 |
| 12.9 | 13.0 | 13.6 | 14.5 | 15.5 |
| 16.0 | 16.4 | 16.9 | 17.4 | 18.1 |
| 18.7 | 19.7 | 19.8 | 20.2 | 21.0 |
| 22.0 | 23.2 | 23.7 | 24.1 | 25.5 |
| 26.0 | 26.6 | 27.5 | 28.2 | 28.9 |
| 30.3 | 31.2 | 32.3 | 33.4 | 35.0 |
| 35.6 | 36.1 | 37.2 | 39.1 | |

TABLE 5
PXRD Diffraction peaks for (R)-1-{2-[4′-(3-methoxy-
propane-1-sulfonyl)-biphenyl-4-yl]-ethyl}-2-methyl-
pyrrolidine di-citrate (degrees 2θ) (from Example 1.5)

| | | | | |
|---|---|---|---|---|
| 5.2 | 7.8 | 10.3 | 11.8 | 12.3 |
| 12.9 | 13.2 | 13.6 | 14.4 | 15.4 |
| 15.6 | 16.0 | 17.0 | 17.4 | 17.5 |
| 17.8 | 18.0 | 18.7 | 19.4 | 19.7 |
| 20.2 | 21.0 | 22.0 | 22.3 | 23.2 |
| 23.6 | 24.1 | 25.22 | 25.4 | 25.9 |
| 27.3 | 28.1 | 28.8 | 30.2 | 32.3 |
| 33.3 | 35.5 | 36.1 | | |

Powder X-ray diffraction peaks for (R)-1-{2-[4′-(3-methoxy-propane-1-sulfonyl)-biphenyl-4-yl]-ethyl}-2-methyl-pyrrolidine maleate are shown in Table 6 below.

TABLE 6
PXRD Diffraction peaks for (R)-1-{2-[4′-(3-
methoxy-propane-1-sulfonyl)-biphenyl-4-yl]-
ethyl}-2-methyl-pyrrolidine maleate (degrees 2θ)

| | | | | |
|---|---|---|---|---|
| 7.9 | 8.5 | 10.3 | 11.9 | 12.7 |
| 15.9 | 16.4 | 16.6 | 17.2 | 18.6 |
| 19.9 | 20.2 | 20.9 | 21.6 | 22.4 |
| 23.6 | 24.5 | 27.5 | 27.9 | 28.8 |
| 32.0 | | | | |

Powder X-ray diffraction peaks for (R)-1-{2-[4′-(3-methoxy-propane-1-sulfonyl)-biphenyl-4-yl]-ethyl}-2-methyl-pyrrolidine hydrochloride are shown in Table 8 below.

TABLE 7
PXRD Diffraction peaks for (R)-1-{2-[4′-(3-
methoxy-propane-1-sulfonyl)-biphenyl-4-yl]-ethyl}-2-
methyl-pyrrolidine hydrochloride (degrees 2θ)

| | | | | |
|---|---|---|---|---|
| 6.9 | 8.7 | 10.2 | 10.7 | 11.6 |
| 13.0 | 13.5 | 14.1 | 14.3 | 16.2 |
| 16.4 | 16.7 | 17.0 | 17.4 | 18.5 |
| 18.7 | 20.4 | 20.9 | 21.5 | 22.0 |
| 22.1 | 22.4 | 25.0 | 25.4 | 25.6 |
| 27.2 | 27.6 | 30.1 | 33.5 | |

### Crystalline form of (R)-1-{2-[4′-(3-methoxy-propane-1-sulfonyl)-biphenyl-4-yl]-ethyl}-2-methyl-pyrrolidine mono-citrate (FORM 1)

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having a powder X-ray diffraction pattern comprising a peak, expressed in terms of 2θ, at about 6.6°. In some embodiments, the crystalline form has a powder X-ray diffraction pattern comprising a peak, expressed in terms of 2θ, at about 13.1°. In some embodiments, the crystalline form has a powder X-ray diffraction pattern comprising a peak, expressed in terms of 2θ, at about 15.8°. In some embodiments, the crystalline form has a powder X-ray diffraction pattern comprising peaks, expressed in terms of 2θ, at about 6.6°, about 13.1°, and about 15.8°. In some embodiments, the crystalline form has a powder X-ray diffraction pattern comprising peaks, expressed in terms of 2θ, at about 6.6°, about 9.9°, about 11.0°, about 13.1°, about 15.8°, about 17.5°, about 18.9°, about 19.6°, and about 23.0°. In some embodiments, the crystalline form has a powder X-ray diffraction pattern substantially as shown in FIG. 1, wherein by “substantially” is meant that the reported peaks can vary by about ±0.2°.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having a differential scanning calorimetry trace comprising endotherms at about 102° C. and about 125° C. In some embodiments, the crystalline form has a differential scanning calorimetry trace comprising a first endotherm with an extrapolated onset temperature between about 80° C. and about 100° C. and a second endotherm with an extrapolated onset temperature between about 105° C. and about 125° C. In some embodiments, the crystalline form has a differential scanning calorimetry trace comprising a first endotherm with an extrapolated onset temperature between about 85° C. and about 95° C. and a second endotherm with an extrapolated onset temperature between about 110° C. and about 120° C. In some embodiments, the crystalline form has a differential scanning calorimetry trace comprising a first endotherm with an extrapolated onset temperature at about 93° C. and a second endotherm with an extrapolated onset temperature at about 115° C. In some embodiments, the crystalline form has a differential scanning calorimetry trace comprising a first endotherm with a peak temperature between about 90° C. and about 110° C. and a second endotherm with a peak temperature between about 115° C. and about 135° C. In some embodiments, the crystalline form has a differential scanning calorimetry trace comprising a first endotherm with a peak temperature at about 102° C. and a second endotherm with a peak temperature at about 125° C. In some embodiments, the crystalline form has a differential scanning calorimetry trace comprising a first endotherm with an associated heat flow of about 45 joules per gram and a second endotherm with an associated heat flow of about 9 joules per gram. In some embodiments, the crystalline form has a differential scanning calorimetry trace comprising a first endotherm with an extrapolated onset temperature at about 93° C., a peak temperature at about 102° C. and an associated heat flow of about 45 joules per gram; and a second endotherm with an extrapolated onset temperature at about 115° C., a peak temperature at about 125° C. and an associated heat flow of about 9 joules per gram. In some embodiments, the crystalline form has a differential scanning calorimetry trace substantially as shown in FIG. 2, wherein by “substantially” is meant that the reported DSC features can vary by about ±4° C.

In some embodiments, the scan rate for the differential scanning calorimetry (DSC) is about 10° C. per minute.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having an infrared absorbance trace comprising peaks at about 1732 cm⁻¹ and about 1587 cm⁻¹. In some embodiments, the crystalline form has an infrared absorbance trace comprising peaks at about 1732 cm⁻¹, about 1587 cm⁻¹, about 1312 cm⁻¹, about 1213 cm⁻¹, and about 1142 cm⁻¹. In some embodiments, the crystalline form has an infrared absorbance trace substantially as shown in FIG. 3, wherein by “substantially” is meant that the reported FTIR peaks can vary by about ±4 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having:
1) an X-ray diffraction pattern comprising peaks, expressed in terms of 2θ, at about 6.6°, about 13.1°, and about 15.8°;
2) a differential scanning calorimetry trace comprising endotherms at about 102° C. and about 125° C.; and
3) an infrared absorbance trace comprising peaks at about 1732 cm⁻¹ and about 1587 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having:
1) an X-ray diffraction pattern comprising peaks, expressed in terms of 2θ, at about 6.6°, about 9.9°, about 11.0°, about 13.1°, about 15.8°, about 17.5°, about 18.9°, about 19.6°, and about 23.0°;
2) a differential scanning calorimetry trace comprising endotherms at about 102° C. and about 125° C.; and
3) an infrared absorbance trace comprising peaks at about 1732 cm⁻¹, about 1587 cm⁻¹, about 1312 cm⁻¹, about 1213 cm⁻¹, and about 1142 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having:
1) an X-ray diffraction pattern comprising peaks, expressed in terms of 2θ, at about 6.6°, about 13.1°, and about 15.8°;
2) a differential scanning calorimetry trace comprising a first endotherm with an extrapolated onset temperature at about 93° C., a peak temperature at about 102° C.; and a second endotherm with an extrapolated onset temperature at about 115° C. and a peak temperature at about 125°; and
3) an infrared absorbance trace comprising peaks at about 1732 cm⁻¹ and about 1587 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having:
1) an X-ray diffraction pattern comprising peaks, expressed in terms of 2θ, at about 6.6°, about 9.9°, about 11.0°, about 13.1°, about 15.8°, about 17.5°, about 18.9°, about 19.6°, and about 23.0°;
2) a differential scanning calorimetry trace comprising a first endotherm with an extrapolated onset temperature at about 93° C., a peak temperature at about 102° C. and an associated heat flow of about 45 joules per gram; and a second endotherm with an extrapolated onset temperature at about 115° C., with a peak temperature at about 125° C. and an associated heat flow of about 9 joules per gram; and
3) an infrared absorbance trace comprising peaks at about 1732 cm⁻¹, about 1587 cm⁻¹, about 1312 cm⁻¹, about 1213 cm⁻¹, and about 1142 cm⁻¹.

### Crystalline form of (R)-1-{2-[4′-(3-methoxy-propane-1-sulfonyl)-biphenyl-4-yl]-ethyl}-2-methyl-pyrrolidine mono-citrate (FORM 2)

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having a powder X-ray diffraction pattern comprising a peak, expressed in terms of 2θ, at about 5.9°. In some embodiments, the crystalline form has a powder X-ray diffraction pattern comprising a peak, expressed in terms of 2θ, at about 9.0°. In some embodiments, the crystalline form has a powder X-ray diffraction pattern comprising a peak, expressed in terms of 2θ, at about 18.9°. In some embodiments, the crystalline form has a powder X-ray diffraction pattern comprising peaks, expressed in terms of 2θ, at about 5.9°, about 9.0°, and about 18.9°. In some embodiments, the crystalline form has a powder X-ray diffraction pattern comprising peaks, expressed in terms of 2θ, about 5.9°, about 6.0°, about 9.0°, about 12.3°, about 14.7°, about 16.2°, about 18.9°, about 19.8°, and about 24.0°. In some embodiments, the crystalline form has a powder X-ray diffraction pattern substantially as shown in FIG. 20, wherein by “substantially” is meant that the reported peaks can vary by about ±0.2°.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having a differential scanning calorimetry trace comprising an endotherm with an extrapolated onset temperature between about 65° C. and about 85° C. In some embodiments, the crystalline form has a differential scanning calorimetry trace comprising an endotherm with an extrapolated onset temperature between about 70° C. and about 80° C. In some embodiments, the crystalline form has a differential scanning calorimetry trace comprising an endotherm with an extrapolated onset temperature at about 76° C. In some embodiments, the crystalline form has a differential scanning calorimetry trace comprising an endotherm with a peak temperature between about 80° C. and about 100° C. In some embodiments, the crystalline form has a differential scanning calorimetry trace comprising an endotherm with a peak temperature at about 88° C. In some embodiments, the crystalline form has a differential scanning calorimetry trace comprising an endotherm with an extrapolated onset temperature at about 76° C. and a peak temperature at about 88°. In some embodiments, the crystalline form has a differential scanning calorimetry trace substantially as shown in FIG. 21, wherein by “substantially” is meant that the reported DSC features can vary by about ±4° C.

In some embodiments, the scan rate for the differential scanning calorimetry (DSC) is about 10° C. per minute.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having a Raman spectrum comprising peaks at about 413 cm⁻¹, about 1140 cm⁻¹ and about 1596 cm⁻¹. In some embodiments, the crystalline form has a Raman spectrum comprising peaks at about 413 cm⁻¹, about 1140 cm⁻¹, about 1284 cm⁻¹, about 1596 cm⁻¹, about 1612 cm⁻¹, about 2935 cm⁻¹ and about 3072 cm⁻¹. In some embodiments, the crystalline form has a Raman spectrum substantially as shown in FIG. 22, wherein by “substantially” is meant that the reported Raman peaks can vary by about ±4 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having:
1) an X-ray diffraction pattern comprising peaks, expressed in terms of 2θ, at about 5.9°, about 9.0°, and about 18.9°.
2) a differential scanning calorimetry trace comprising an endotherm with an extrapolated onset temperature at about 76° C. and a peak temperature at about 88° C.; and
3) a Raman spectrum comprising peaks at about 413 cm⁻¹, about 1140 cm⁻¹ and about 1596 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine mono-citrate having:
1) an X-ray diffraction pattern comprising peaks, expressed in terms of 2θ, at about at about 5.9°, about 6.0°, about 9.0°, about 12.3°, about 14.7°, about 16.2°, about 18.9°, about 19.8°, and about 24.0°;
2) a differential scanning calorimetry trace comprising an endotherm with an extrapolated onset temperature at about 76° C. and a peak temperature at about 88° C.; and
3) a Raman spectrum comprising peaks at 413 cm⁻¹, about 1140 cm⁻¹, about 1284 cm⁻¹, about 1596 cm⁻¹, about 1612 cm⁻¹, about 2935 cm⁻¹ and about 3072 cm⁻¹.

### Crystalline form of (R)-1-{2-[4′-(3-methoxy-propane-1-sulfonyl)-biphenyl-4-yl]-ethyl}-2-methyl-pyrrolidine di-citrate (from Example 1.2).

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having an X-ray diffraction pattern comprising a peak, expressed in terms of 2θ, at about 7.8°. In some embodiments, the crystalline form has an X-ray diffraction pattern comprising a peak, expressed in terms of 2θ, at about 10.3°. In some embodiments, the crystalline form has an X-ray diffraction pattern comprising a peak, expressed in terms of 2θ, at about 15.5°. In some embodiments, the crystalline form has an X-ray diffraction pattern comprising peaks, expressed in terms of 2θ, at about 7.8°, about 10.3°, and about 15.5°. In some embodiments, the crystalline form has an X-ray diffraction pattern comprising peaks, expressed in terms of 2θ, at about 7.8°, about 10.3°, about 11.8°, about 12.9°, about 13.6°, about 15.5°, about 18.1°, about 18.7°, about 19.7°, about 20.2°, about 22.0°, and about 23.2°. In some embodiments, the crystalline form has an X-ray diffraction pattern substantially as shown in FIG. 5, wherein by “substantially” is meant that the reported peaks can vary by about ±0.2°.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having a differential scanning calorimetry trace comprising an endotherm at about 149° C. In some embodiments, the crystalline form has a differential scanning calorimetry trace comprising an endotherm with an extrapolated onset temperature between about 135° C. and about 155° C. In some embodiments, the crystalline form has a differential scanning calorimetry trace comprising an endotherm with an extrapolated onset temperature between about 140° C. and about 150° C. In some embodiments, the crystalline form has a differential scanning calorimetry trace comprising an endotherm with an extrapolated onset temperature at about 145° C. In some embodiments, the crystalline form has a differential scanning calorimetry trace comprising an endotherm with a peak temperature between about 140° C. and about 160° C. In some embodiments, the crystalline form has a differential scanning calorimetry trace comprising an endotherm with a peak temperature at about 149° C. In some embodiments, the crystalline form has an associated heat flow of about 106 joules per gram. In some embodiments, the crystalline form has a differential scanning calorimetry trace comprising an endotherm with an extrapolated onset temperature at about 145° C., a peak temperature at about 149° C. and an associated heat flow of about 106 joules per gram. In some embodiments, the crystalline form has a differential scanning calorimetry trace substantially as shown in FIG. 6, wherein by “substantially” is meant that the reported DSC features can vary by about ±4° C.

In some embodiments, the scan rate for the differential scanning calorimetry (DSC) is about 10° C. per minute.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having an infrared absorbance trace comprising peaks at about 1738 cm⁻¹, about 1726 cm⁻¹, and about 1686 cm⁻¹. In some embodiments, the crystalline form has an infrared absorbance trace comprising peaks at about 1738 cm⁻¹, about 1726 cm⁻¹, about 1686 cm⁻¹, about 1304 cm⁻¹, about 1213 cm⁻¹, and about 1146 cm⁻¹. In some embodiments, the crystalline form has an infrared absorbance trace substantially as shown in FIG. 7, wherein by “substantially” is meant that the reported FTIR peaks can vary by about ±4 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having a dynamic vapor sorption profile substantially as shown in FIG. 8, wherein by “substantially” is meant that the reported DVS features can vary by about ±5% RH.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.8°, about 10.3°, and about 15.5°;
2) a differential scanning calorimetry trace comprising an endotherm at about 149° C.; and
3) an infrared absorbance trace comprising peaks at about 1738 cm⁻¹, about 1726 cm⁻¹, and about 1686 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.8°, about 10.3°, about 11.8°, about 12.9°, about 13.6°, about 15.5°, about 18.1°, about 18.7°, about 19.7°, about 20.2°, about 22.0°, and about 23.2°;
2) a differential scanning calorimetry trace comprising an endotherm at about 149° C.; and
3) an infrared absorbance trace comprising peaks at about 1738 cm⁻¹, about 1726 cm⁻¹, about 1686 cm⁻¹, about 1304 cm⁻¹, about 1213 cm⁻¹, and about 1146 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.8°, about 10.3°, about 11.8°, about 12.9°, about 13.6°, about 15.5°, about 18.1°, about 18.7°, about 19.7°, about 20.2°, about 22.0°, and about 23.2°;
2) a differential scanning calorimetry trace comprising an endotherm at about 149° C.;
3) an infrared absorbance trace comprising peaks at about 1738 cm⁻¹, about 1726 cm⁻¹, about 1686 cm⁻¹, about 1304 cm⁻¹, about 1213 cm⁻¹, and about 1146 cm⁻¹; and
4) a dynamic vapor sorption profile substantially as shown in FIG. 8, wherein by “substantially” is meant that the reported DVS features can vary by about ±5% RH.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.8°, about 10.3°, and about 15.5°;
2) a differential scanning calorimetry trace comprising an endotherm with an extrapolated onset temperature at about 145° C. and a peak temperature at about 149° C.; and
3) an infrared absorbance trace comprising peaks at about 1738 cm⁻¹, about 1726 cm⁻¹, and about 1686 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.8°, about 10.3°, about 11.8°, about 12.9°, about 13.6°, about 15.5°, about 18.1°, about 18.7°, about 19.7°, about 20.2°, about 22.0°, and about 23.2°;
2) a differential scanning calorimetry trace comprising an endotherm with an extrapolated onset temperature at about 145° C., a peak temperature at about 149° C., and an associated heat flow of about 106 joules per gram; and
3) an infrared absorbance trace comprising peaks at about 1738 cm⁻¹, about 1726 cm⁻¹, about 1686 cm⁻¹, about 1304 cm⁻¹, about 1213 cm⁻¹, and about 1146 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.8°, about 10.3°, about 11.8°, about 12.9°, about 13.6°, about 15.5°, about 18.1°, about 18.7°, about 19.7°, about 20.2°, about 22.0°, and about 23.2°;
2) a differential scanning calorimetry trace comprising an endotherm with an extrapolated onset temperature of about 145° C., a peak temperature of about 149° C., and an associated heat flow of about 106 joules per gram; and
3) an infrared absorbance trace comprising peaks at about 1738 cm⁻¹, about 1726 cm⁻¹, about 1686 cm⁻¹, about 1304 cm⁻¹, about 1213 cm⁻¹, and about 1146 cm⁻¹; and
4) a dynamic vapor sorption profile substantially as shown in FIG. 8.

### Crystalline form of (R)-1-{2-[4′-(3-methoxy-propane-1-sulfonyl)-biphenyl-4-yl]-ethyl}-2-methyl-pyrrolidine di-citrate (from Example 1.5).

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having a powder X-ray diffraction pattern comprising a peak, expressed in terms of 2θ, at about 7.7°. In some embodiments, the crystalline form has a powder X-ray diffraction pattern comprising a peak, expressed in terms of 2θ, at about 11.8°. In some embodiments, the crystalline form has a powder X-ray diffraction pattern comprising a peak, expressed in terms of 2θ, at about 18.7°. In some embodiments, the crystalline form has a powder X-ray diffraction pattern comprising peaks, expressed in terms of 2θ, at about 7.7°, about 11.8°, and about 18.7°. In some embodiments, the crystalline form has a powder X-ray diffraction pattern comprising peaks, expressed in terms of 2θ, at about 7.7°, about 10.3°, about 11.8°, about 12.9°, about 13.6°, about 15.4°, about 18.0°, about 18.7°, about 19.7°, about 20.2°, about 22.0°, and about 23.2°. In some embodiments, the crystalline form has a powder X-ray diffraction pattern substantially as shown in FIG. 16, wherein by “substantially” is meant that the reported peaks can vary by about ±0.2°.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having a differential scanning calorimetry trace comprising an endotherm with an extrapolated onset temperature between about 140° C. and about 160° C. In some embodiments, the crystalline form has a differential scanning calorimetry trace comprising an endotherm with an extrapolated onset temperature between about 145° C. and about 155° C. In some embodiments, the crystalline form has a differential scanning calorimetry trace comprising an endotherm with an extrapolated onset temperature at about 150° C. In some embodiments, the crystalline form has a differential scanning calorimetry trace comprising an endotherm with a peak temperature between about 145° C. and about 155° C. In some embodiments, the crystalline form has a differential scanning calorimetry trace comprising an endotherm with a peak temperature at about 151° C. In some embodiments, the crystalline form has a differential scanning calorimetry trace comprising an endotherm with an extrapolated onset temperature at about 150° C. and a peak temperature at about 151°. In some embodiments, the crystalline form has a differential scanning calorimetry trace substantially as shown in FIG. 17, wherein by “substantially” is meant that the reported DSC features can vary by about ±4° C.

In some embodiments, the scan rate for the differential scanning calorimetry (DSC) is about 10° C. per minute.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having a Raman spectrum comprising peaks at about 746 cm⁻¹, about 1596 cm⁻¹ and about 2963 cm⁻¹. In some embodiments, the crystalline form has a Raman spectrum comprising peaks at about 416 cm⁻¹, about 746 cm⁻¹, about 788 cm⁻¹, about 1284 cm⁻¹, about 1596 cm⁻¹, about 1612 cm⁻¹, about 2963 cm⁻¹ and about 3073 cm⁻¹. In some embodiments, the crystalline form has a Raman spectrum substantially as shown in FIG. 19, wherein by “substantially” is meant that the reported Raman peaks can vary by about ±4 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.8°, about 10.3°, and about 15.5°;
2) a differential scanning calorimetry trace comprising an endotherm with an extrapolated onset temperature at about 150° C. and a peak temperature at about 151° C.; and
3) a Raman spectrum comprising peaks at about 746 cm⁻¹, about 1596 cm⁻¹, and about 2963 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.8°, about 10.3°, about 11.8°, about 12.9°, about 13.6°, about 15.5°, about 18.1°, about 18.7°, about 19.7°, about 20.2°, about 22.0°, and about 23.2°;
2) a differential scanning calorimetry trace comprising an endotherm with an extrapolated onset temperature between about 145° C. and about 155° C. and a peak temperature between about 145° C. and about 155° C.; and
3) a Raman spectrum comprising peaks at about 416 cm⁻¹, about 746 cm⁻¹, about 788 cm⁻¹, about 1284 cm⁻¹, about 1596 cm⁻¹, about 1612 cm⁻¹, about 2963 cm⁻¹ and about 3073 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.8°, about 10.3°, about 11.8°, about 12.9°, about 13.6°, about 15.5°, about 18.1°, about 18.7°, about 19.7°, about 20.2°, about 22.0°, and about 23.2°;
2) a differential scanning calorimetry trace comprising an endotherm with an extrapolated onset temperature at about 150° C. and a peak temperature at about 151° C.; and
3) a Raman spectrum comprising peaks at about 416 cm⁻¹, about 746 cm⁻¹, about 788 cm⁻¹, about 1284 cm⁻¹, about 1596 cm⁻¹, about 1612 cm⁻¹, about 2963 cm⁻¹ and about 3073 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.7°, about 11.8°, and about 18.7°;
2) a differential scanning calorimetry trace comprising an endotherm with an extrapolated onset temperature at about 150° C. and a peak temperature at about 151° C.; and
3) a Raman spectrum comprising peaks at about 746 cm⁻¹, about 1596 cm⁻¹, and about 2963 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine di-citrate having:
1) an X-ray diffraction pattern comprising peaks, expressed in terms of 2θ, at about 7.7°, about 10.3°, about 11.8°, about 12.9°, about 13.6°, about 15.4°, about 18.0°, about 18.7°, about 19.7°, about 20.2°, about 22.0°, and about 23.2°;
2) a differential scanning calorimetry trace comprising an endotherm with an extrapolated onset temperature at about 150° C. and a peak temperature at about 151° C.; and
3) a Raman spectrum comprising peaks at about 416 cm⁻¹, about 746 cm⁻¹, about 788 cm⁻¹, about 1284 cm⁻¹, about 1596 cm⁻¹, about 1612 cm⁻¹, about 2963 cm⁻¹ and about 3073 cm⁻¹.

### Crystalline form of (R)-1-{2-[4′-(3-methoxy-propane-1-sulfonyl)-biphenyl-4-yl]-ethyl}-2-methyl-pyrrolidine maleate

One aspect of the present invention is directed to a crystalline form of (R)-1-{2-[4′-(3-methoxy-propane-1-sulfonyl)-biphenyl-4-yl]-ethyl}-2-methyl-pyrrolidine maleate having a powder X-ray diffraction pattern comprising a peak, expressed in terms of 2θ, at about 7.9°. In some embodiments, the crystalline form has a powder X-ray diffraction pattern comprising a peak, expressed in terms of 2θ, at about 11.9°. In some embodiments, the crystalline form has a powder X-ray diffraction pattern comprising a peak, expressed in terms of 2θ, at about 17.2°. In some embodiments, the crystalline form has a powder X-ray diffraction pattern comprising a peak, expressed in terms of 2θ, at about 19.9°. In some embodiments, the crystalline form has a powder X-ray diffraction pattern comprising peaks, expressed in terms of 2θ, at about 7.9°, about 11.9°, about 17.2° and about 19.9°. In some embodiments, the crystalline form has a powder X-ray diffraction pattern comprising peaks, expressed in terms of 2θ, at about 7.9°, about 8.5°, about 10.3°, about 11.9°, about 12.7°, about 15.9°, about 17.2°, about 18.6°, about 19.9°, about 21.6°, and about 22.4°. In some embodiments, the crystalline form has a powder X-ray diffraction pattern substantially free of any peaks, expressed in terms of 2θ, between 13.3° and 15.3°. In some embodiments, the crystalline form has a powder X-ray diffraction pattern substantially as shown in FIG. 12, wherein by “substantially” is meant that the reported peaks can vary by about ±0.2°.

One aspect of the present invention is directed to a crystalline form of (R)-1-{2-[4′-(3-methoxy-propane-1-sulfonyl)-biphenyl-4-yl]-ethyl}-2-methyl-pyrrolidine maleate having a differential scanning calorimetry trace comprising an endotherm with an extrapolated onset temperature between about 110° C. and about 130° C. In some embodiments, the crystalline form has a differential scanning calorimetry trace comprising an endotherm with an extrapolated onset temperature between about 115° C. and about 125° C. In some embodiments, the crystalline form has a differential scanning calorimetry trace comprising an endotherm with an extrapolated onset temperature at about 121° C. In some embodiments, the crystalline form has a differential scanning calorimetry trace comprising an endotherm with a peak temperature between about 112° C. and about 132° C. In some embodiments, the crystalline form has a differential scanning calorimetry trace comprising an endotherm with a peak temperature at about 122° C. In some embodiments, the crystalline form has an associated heat flow of about 72 joules per gram. In some embodiments, the crystalline form has a differential scanning calorimetry trace comprising an endotherm with an extrapolated onset temperature at about 121° C., a peak temperature at about 122° and an associated heat flow of about 72 joules per gram. In some embodiments, the crystalline form has a differential scanning calorimetry trace substantially as shown in FIG. 13, wherein by “substantially” is meant that the reported DSC features can vary by about ±4° C.

In some embodiments, the scan rate for the differential scanning calorimetry (DSC) is about 10° C. per minute.

One aspect of the present invention is directed to a crystalline form of (R)-1-{2-[4′-(3-methoxy-propane-1-sulfonyl)-biphenyl-4-yl]-ethyl}-2-methyl-pyrrolidine maleate having a Raman spectrum comprising peaks at about 423 cm⁻¹, about 1598 cm⁻¹ and about 2985 cm⁻¹. In some embodiments, the crystalline form has a Raman spectrum comprising peaks at about 423 cm⁻¹, about 883 cm⁻¹, about 1017 cm⁻¹, about 1089 cm⁻¹, about 1283 cm⁻¹, about 1598 cm⁻¹, about 1613 cm⁻¹, about 2985 cm⁻¹, about 3045 cm⁻¹ and about 3072 cm⁻¹. In some embodiments, the crystalline form has a Raman spectrum substantially as shown in FIG. 15, wherein by “substantially” is meant that the reported Raman peaks can vary by about ±4 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine maleate having:
1) an X-ray diffraction pattern comprising peaks, expressed in terms of 2θ, at about 7.9°, about 11.9°, about 17.2°, and about 19.9°;
2) a differential scanning calorimetry trace comprising an endotherm with an extrapolated onset temperature between about 110° C. and about 130° C. and a peak temperature between about 112° C. and about 132° C.; and
3) a Raman spectrum comprising peaks at about 423 cm⁻¹, about 1598 cm⁻¹ and about 2985 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine maleate having:
1) an X-ray diffraction pattern comprising peaks, expressed in terms of 2θ, at about 7.9°, about 8.5°, about 10.3°, about 11.9°, about 12.7°, about 15.9°, about 17.2°, about 18.6°, about 19.9°, about 21.6°, and about 22.4°;
2) a differential scanning calorimetry trace comprising an endotherm with an extrapolated onset temperature between about 110° C. and about 130° C., a peak temperature between about 112° C. and about 132° C. and an associated heat flow of 72 joules per gram; and
3) a Raman spectrum comprising peaks at about 423 cm⁻¹, about 883 cm⁻¹, about 1017 cm⁻¹, about 1089 cm⁻¹, about 1283 cm⁻¹, about 1598 cm⁻¹, about 1613 cm⁻¹, about 2985 cm⁻¹, about 3045 cm⁻¹ and about 3072 cm⁻¹.

### Crystalline form of (R)-1-{2-[4′-(3-methoxy-propane-1-sulfonyl)-biphenyl-4-yl]-ethyl}-2-methyl-pyrrolidine hydrochloride

One aspect of the present invention is directed to a crystalline form of (R)-1-{2-[4′-(3-methoxy-propane-1-sulfonyl)-biphenyl-4-yl]-ethyl}-2-methyl-pyrrolidine hydrochloride having a powder X-ray diffraction pattern comprising a peak, expressed in terms of 2θ, at about 8.7°. In some embodiments, the crystalline form has a powder X-ray diffraction pattern comprising a peak, expressed in terms of 2θ, at about 14.1°. In some embodiments, the crystalline form has a powder X-ray diffraction pattern comprising a peak, expressed in terms of 2θ, at about 16.4°. In some embodiments, the crystalline form has a powder X-ray diffraction pattern comprising peaks, expressed in terms of 2θ, at about 8.7°, about 14.1°, and about 16.4°. In some embodiments, the crystalline form has a powder X-ray diffraction pattern comprising peaks, expressed in terms of 2θ, at about 6.9°, about 8.7°, about 10.7°, about 11.6°, about 13.0°, about 14.1°, about 16.2°, about 16.4°, about 16.7°, about 17.0°, about 20.9°, and about 25.6°. In some embodiments, the crystalline form has a powder X-ray diffraction pattern substantially as shown in FIG. 23, wherein by “substantially” is meant that the reported peaks can vary by about ±0.2°.

One aspect of the present invention is directed to a crystalline form of (R)-1-{2-[4′-(3-methoxy-propane-1-sulfonyl)-biphenyl-4-yl]-ethyl}-2-methyl-pyrrolidine hydrochloride having a differential scanning calorimetry trace comprising an endotherm with an extrapolated onset temperature between about 100° C. and about 120° C. In some embodiments, the crystalline form has a differential scanning calorimetry trace comprising an endotherm with an extrapolated onset temperature between about 105° C. and about 115° C. In some embodiments, the crystalline form has a differential scanning calorimetry trace comprising an endotherm with an extrapolated onset temperature at about 111° C. In some embodiments, the crystalline form has a differential scanning calorimetry trace comprising an endotherm with a peak temperature between about 103° C. and about 123° C. In some embodiments, the crystalline form has a differential scanning calorimetry trace comprising an endotherm with a peak temperature at about 114° C. In some embodiments, the crystalline form has an associated heat flow of about 63 joules per gram. In some embodiments, the crystalline form has a differential scanning calorimetry trace comprising an endotherm with an extrapolated onset temperature at about 111° C., a peak temperature at about 114° and an associated heat flow of about 63 joules per gram. In some embodiments, the crystalline form has a differential scanning calorimetry trace substantially as shown in FIG. 24, wherein by “substantially” is meant that the reported DSC features can vary by about ±4° C.

In some embodiments, the scan rate for the differential scanning calorimetry (DSC) is about 10° C. per minute.

One aspect of the present invention is directed to a crystalline form of (R)-1-{2-[4′-(3-methoxy-propane-1-sulfonyl)-biphenyl-4-yl]-ethyl}-2-methyl-pyrrolidine hydrochloride having a Raman spectrum comprising peaks at about 417 cm⁻¹, about 1599 cm⁻¹ and about 2920 cm⁻¹. In some embodiments, the crystalline form has a Raman spectrum comprising peaks at about 418 cm⁻¹, about 629 cm⁻¹, about 748 cm⁻¹, about 843 cm⁻¹, about 1090 cm⁻¹, about 1151 cm⁻¹, about 1196 cm⁻¹, about 1290 cm⁻¹, about 1523 cm⁻, about 1597 cm⁻¹, about 1612 cm⁻¹, about 2817 cm⁻¹, about 2920 cm⁻¹ and about 3066 cm⁻¹. In some embodiments, the crystalline form has a Raman spectrum substantially as shown in FIG. 26, wherein by “substantially” is meant that the reported Raman peaks can vary by about ±4 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine hydrochloride having:
1) an X-ray diffraction pattern comprising peaks, expressed in terms of 2θ, at about 8.7°, about 14.1° and about 16.4°;
2) a differential scanning calorimetry trace comprising an endotherm with an extrapolated onset temperature between about 100° C. and about 120° C. and a peak temperature between about 103° C. and about 123° C.; and
3) a Raman spectrum comprising peaks at about 418 cm⁻¹, about 1597 cm⁻¹ and about 2920 cm⁻¹.

One aspect of the present invention is directed to a crystalline form of (R)-1-{2-[4′-(3-methoxy-propane-1-sulfonyl)-biphenyl-4-yl]-ethyl}-2-methyl-pyrrolidine hydrochloride having:
1) an X-ray diffraction pattern comprising peaks, expressed in terms of 2θ, at about 6.9°, about 8.7°, about 10.7°, about 11.6°, about 13.0°, about 14.1°, about 16.2°, about 16.4°, about 16.7°, about 17.0°, about 20.9°, and about 25.6°;
2) a differential scanning calorimetry trace comprising an endotherm with an extrapolated onset temperature between about 100° C. and about 120° C., a peak temperature between about 103° C. and about 123° C. and an associated heat flow of 63 joules per gram; and
3) a Raman spectrum comprising peaks at about 418 cm⁻¹, about 629 cm⁻¹, about 748 cm⁻¹, about 843 cm⁻¹, about 1090 cm⁻¹, about 1151 cm⁻¹, about 1196 cm⁻¹, about 1290 cm⁻¹, about 1523 cm⁻¹, about 1597 cm⁻¹, about 1612 cm⁻¹, about 2817 cm⁻¹, about 2920 cm⁻¹ and about 3066 cm⁻¹.

### Compositions and Pharmaceutical Compositions

One aspect of the present invention is directed to compositions comprising (R)-1-{2-[4′-(3-methoxy-propane-1-sulfonyl)-biphenyl-4-yl]-ethyl}-2-methyl-pyrrolidine mono and/or dicitrate.

One aspect of the present invention is directed to compositions comprising (R)-1-{2-[4′-(3-methoxy-propane-1-sulfonyl)-biphenyl-4-yl]-ethyl}-2-methyl-pyrrolidine maleate.

One aspect of the present invention is directed to compositions comprising (R)-1-{2-[4′-(3-methoxy-propane-1-sulfonyl)-biphenyl-4-yl]-ethyl}-2-methyl-pyrrolidine hydrochloride.

Compositions of the present invention also embrace pharmaceutical compositions. Accordingly, some embodiments of the present invention are directed to pharmaceutical compositions comprising (R)-1-{2-[4′-(3-methoxy-propane-1-sulfonyl)-biphenyl-4-yl]-ethyl}-2-methyl-pyrrolidine mono-citrate and a pharmaceutically acceptable carrier.

Some embodiments of the present invention are directed to pharmaceutical compositions comprising a crystalline form of (R)-1-{2-[4′-(3-methoxy-propane-1-sulfonyl)-biphenyl-4-yl]-ethyl}-2-methyl-pyrrolidine mono-citrate, as described herein, and a pharmaceutically acceptable carrier.

Some embodiments of the present invention are directed to pharmaceutical compositions comprising (R)-1-{2-[4′-(3-methoxy-propane-1-sulfonyl)-biphenyl-4-yl]-ethyl}-2-methyl-pyrrolidine di-citrate and a pharmaceutically acceptable carrier.

Some embodiments of the present invention are directed to pharmaceutical compositions comprising a crystalline form of (R)-1-{2-[4′-(3-methoxy-propane-1-sulfonyl)-biphenyl-4-yl]-ethyl}-2-methyl-pyrrolidine di-citrate, as described herein, and a pharmaceutically acceptable carrier.

Some embodiments of the present invention are directed to pharmaceutical compositions comprising (R)-1-{2-[4′-(3-methoxy-propane-1-sulfonyl)-biphenyl-4-yl]-ethyl}-2-methyl-pyrrolidine maleate and a pharmaceutically acceptable carrier.

Some embodiments of the present invention are directed to pharmaceutical compositions comprising a crystalline form of (R)-1-{2-[4′-(3-methoxy-propane-1-sulfonyl)-biphenyl-4-yl]-ethyl}-2-methyl-pyrrolidine maleate, as described herein, and a pharmaceutically acceptable carrier.

Some embodiments of the present invention are directed to pharmaceutical compositions comprising (R)-1-{2-[4′-(3-methoxy-propane-1-sulfonyl)-biphenyl-4-yl]-ethyl}-2-methyl-pyrrolidine hydrochloride and a pharmaceutically acceptable carrier.

Some embodiments of the present invention are directed to pharmaceutical compositions comprising a crystalline form of (R)-1-{2-[4′-(3-methoxy-propane-1-sulfonyl)-biphenyl-4-yl]-ethyl}-2-methyl-pyrrolidine hydrochloride, as described herein, and a pharmaceutically acceptable carrier.

The present invention further provides compositions and/or pharmaceutical compositions comprising (R)-1-{2-[4′-(3-methoxy-propane-1-sulfonyl)-biphenyl-4-yl]-ethyl}-2-methyl-pyrrolidine mono-citrate.

The present invention further provides compositions and/or pharmaceutical compositions comprising the crystalline form of (R)-1-{2-[4′-(3-methoxy-propane-1-sulfonyl)-biphenyl-4-yl]-ethyl}-2-methyl-pyrrolidine mono-citrate.

The present invention further provides compositions and/or pharmaceutical compositions comprising (R)-1-{2-[4′-(3-methoxy-propane-1-sulfonyl)-biphenyl-4-yl]-ethyl}-2-methyl-pyrrolidine di-citrate.

The present invention further provides compositions and/or pharmaceutical compositions comprising crystalline form of (R)-1-{2-[4′-(3-methoxy-propane-1-sulfonyl)-biphenyl-4-yl]-ethyl}-2-methyl-pyrrolidine di-citrate.

The present invention further provides compositions and/or pharmaceutical compositions comprising (R)-1-{2-[4′-(3-methoxy-propane-1-sulfonyl)-biphenyl-4-yl]-ethyl}-2-methyl-pyrrolidine maleate.

The present invention further provides compositions and/or pharmaceutical compositions comprising a crystalline form of (R)-1-{2-[4′-(3-methoxy-propane-1-sulfonyl)-biphenyl-4-yl]-ethyl}-2-methyl-pyrrolidine maleate.

The present invention further provides compositions and/or pharmaceutical compositions comprising (R)-1-{2-[4′-(3-methoxy-propane-1-sulfonyl)-biphenyl-4-yl]-ethyl}-2-methyl-pyrrolidine hydrochloride.

The present invention further provides compositions and/or pharmaceutical compositions comprising a crystalline form of (R)-1-{2-[4′-(3-methoxy-propane-1-sulfonyl)-biphenyl-4-yl]-ethyl}-2-methyl-pyrrolidine hydrochloride.

In some embodiments, the compositions and/or pharmaceutical compositions of the invention comprise about 1, about 2.5, about 5, about 7.5, about 10, about 15, about 20, about 25, about 50, about 60, about 70, about 75, about 80, about 85, about 90, about 95, or about 95% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 1, about 2.5, about 5, about 7.5, about 10, about 15, about 20, about 25, about 50, about 60, about 70, about 75, about 80, about 85, about 90, about 95, or about 95% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 1, about 2.5, about 5, about 7.5, about 10, about 15, about 20, about 25, about 50, about 60, about 70, about 75, about 80, about 85, about 90, about 95, or about 95% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 1, about 2.5, about 5, about 7.5, about 10, about 15, about 20, about 25, about 50, about 60, about 70, about 75, about 80, about 85, about 90, about 95, or about 95% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 1% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 5% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 10% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 20% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 25% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 50% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 75% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 80% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 90% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions zof the invention comprise about 95% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 99% or greater by weight of (R)-1-{2-[4′-(3-methoxy-propane-1-sulfonyl)-biphenyl-4-yl]-ethyl}-2-methyl-pyrrolidine mono-citrate and/or a crystalline form thereof.

In some embodiments, the compositions and/or pharmaceutical compositions of the invention comprise about 1% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 5% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 10% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 20% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 25% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 50% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 75% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 80% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 90% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 95% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 99% or greater by weight of (R)-1-{2-[4′-(3-methoxy-propane-1-sulfonyl)-biphenyl-4-yl]-ethyl}-2-methyl-pyrrolidine di-citrate and/or a crystalline form thereof.

In some embodiments, the compositions and/or pharmaceutical compositions of the invention comprise about 1% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 5% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 10% or greater by weight of (R)-1-{2-[4′-(3-methoxy-propane-1-sulfonyl)-biphenyl-4-yl]-ethyl}-2-methyl-pyrrolidine mono-maleate and/or a crystalline form thereof.

In some embodiments, the compositions and/or pharmaceutical compositions of the invention comprise about 20% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 25% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 50% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 75% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 80% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 90% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 95% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 99% or greater by weight of (R)-1-{2-[4′-(3-methoxy-propane-1-sulfonyl)-biphenyl-4-yl]-ethyl}-2-methyl-pyrrolidine maleate and/or a crystalline form thereof.

In some embodiments, the compositions and/or pharmaceutical compositions of the invention comprise about 1% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 5% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 10% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 20% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 25% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 50% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 75% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 80% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 90% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 95% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

In some embodiments, the compositions and/or pharmaceutical compositions of the invention comprise about 99% or greater by weight of (R)-1-{2-[4′-(3-methoxy-propane-1-sulfonyl)-biphenyl-4-yl]-ethyl}-2-methyl-pyrrolidine hydrochloride and/or a crystalline form thereof.

### Pharmaceutical Compositions

Pharmaceutical compositions may be prepared by any suitable method, typically by uniformly mixing the active compound(s) with liquids or finely divided solid carriers, or both, in the required proportions, and then, if necessary, forming the resulting mixture into a desired shape.

Accordingly, another aspect of the present invention is directed to processes for preparing pharmaceutical compositions comprising admixing a citrate salt of (R)-1-{2-[4′-(3-methoxy-propane-1-sulfonyl)-biphenyl-4-yl]-ethyl}-2-methyl-pyrrolidine and/or a crystalline form thereof, as described herein, and a pharmaceutically acceptable carrier.

Some embodiments are directed to processes for preparing pharmaceutical compositions comprising admixing (R)-1-{2-[4′-(3-methoxy-propane-1-sulfonyl)-biphenyl-4-yl]-ethyl}-2-methyl-pyrrolidine mono-citrate and a pharmaceutically acceptable carrier.

Some embodiments are directed to processes for preparing pharmaceutical compositions comprising admixing a crystalline form of (R)-1-{2-[4′-(3-methoxy-propane-1-sulfonyl)-biphenyl-4-yl]-ethyl}-2-methyl-pyrrolidine mono-citrate, as described herein, and a pharmaceutically acceptable carrier.

Some embodiments are directed to processes for preparing pharmaceutical compositions comprising admixing (R)-1-{2-[4′-(3-methoxy-propane-1-sulfonyl)-biphenyl-4-yl]-ethyl}-2-methyl-pyrrolidine di-citrate and a pharmaceutically acceptable carrier.

Some embodiments are directed to processes for preparing pharmaceutical compositions comprising admixing a crystalline form of (R)-1-{2-[4′-(3-methoxy-propane-1-sulfonyl)-biphenyl-4-yl]-ethyl}-2-methyl-pyrrolidine di-citrate, as described herein, and a pharmaceutically acceptable carrier.

Another aspect of the present invention is directed to processes for preparing pharmaceutical compositions comprising admixing a maleate salt of (R)-1-{2-[4′-(3-methoxy-propane-1-sulfonyl)-biphenyl-4-yl]-ethyl}-2-methyl-pyrrolidine and/or a crystalline form thereof, as described herein, and a pharmaceutically acceptable carrier.

Some embodiments are directed to processes for preparing pharmaceutical compositions comprising admixing (R)-1-{2-[4′-(3-methoxy-propane-1-sulfonyl)-biphenyl-4-yl]-ethyl}-2-methyl-pyrrolidine maleate and a pharmaceutically acceptable carrier.

Some embodiments are directed to processes for preparing pharmaceutical compositions comprising admixing a crystalline form of (R)-1-{2-[4′-(3-methoxy-propane-1-sulfonyl)-biphenyl-4-yl]-ethyl}-2-methyl-pyrrolidine maleate, as described herein, and a pharmaceutically acceptable carrier.

Another aspect of the present invention is directed to processes for preparing pharmaceutical compositions comprising admixing a hydrochloride salt of (R)-1-{2-[4′-(3-methoxy-propane-1-sulfonyl)-biphenyl-4-yl]-ethyl}-2-methyl-pyrrolidine and/or a crystalline form thereof, as described herein, and a pharmaceutically acceptable carrier.

Some embodiments are directed to processes for preparing pharmaceutical compositions comprising admixing (R)-1-{2-[4′-(3-methoxy-propane-1-sulfonyl)-biphenyl-4-yl]-ethyl}-2-methyl-pyrrolidine hydrochloride and a pharmaceutically acceptable carrier.

Some embodiments are directed to processes for preparing pharmaceutical compositions comprising admixing a crystalline form of (R)-1-{2-[4′-(3-methoxy-propane-1-sulfonyl)-biphenyl-4-yl]-ethyl}-2-methyl-pyrrolidine hydrochloride, as described herein, and a pharmaceutically acceptable carrier.

Conventional excipients, such as binding agents, fillers, acceptable wetting agents, tabletting lubricants, and disintegrants may be used in tablets and capsules for oral administration. Liquid preparations for oral administration may be in the form of solutions, emulsions, aqueous or oily suspensions, and syrups. Alternatively, the oral preparations may be in the form of a dry powder that can be reconstituted with water or another suitable liquid vehicle before use. Additional additives such as suspending or emulsifying agents, non-aqueous vehicles (including edible oils), preservatives, and flavorings and colorants may be added to the liquid preparations. Parenteral dosage forms may be prepared by dissolving the compound of the invention in a suitable liquid vehicle and filter sterilizing the solution before filling and sealing an appropriate vial or ampoule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Ed., 2000, Lippincott Williams & Wilkins, (Editors: Gennaro, A. R., et al).

While it is possible that a compound or crystalline form thereof as described herein may, in an alternative use, be administered as a raw or pure chemical, it is preferable however to present the compound or active ingredient as a pharmaceutical formulation or composition further comprising a pharmaceutically acceptable carrier.

The invention thus further provides pharmaceutical formulations comprising a compound of the invention or a pharmaceutically acceptable salt or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof.

The invention thus further provides pharmaceutical formulations comprising a salt of the invention or a solvate, hydrate or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Brittan, Vol. 95, Marcel Dekker, Inc., New York, 1999, incorporated herein by reference in its entirety.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof, and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethylcellulose; and with lubricants such as talc or magnesium stearate. The active ingredient may also be administered by injection as a composition wherein, for example, saline, dextrose or water may be used as a suitable pharmaceutically acceptable carrier.

The dose when using the compounds of the present invention can vary within wide limits, as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present invention. Representative doses of the present invention include, but are not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg, and about 0.001 mg to about 25 mg. Multiple doses may be administered during the day, especially when relatively large amounts are deemed to be needed, for example 2, 3 or 4, doses. Depending on the individual and as deemed appropriate from the patient's physician or care-giver it may be necessary to deviate upward or downward from the doses described herein.

The amount of active ingredient, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, whether the disease state is chronic or acute, whether treatment or prophylaxis is conducted, or on whether further active compounds are administered in addition to the compounds of the present invention and as part of a drug combination. The dosage regimen for treating a disease condition with the compounds and/or compositions of this invention is selected in accordance with a variety of factors as cited above. Thus, the actual dosage regimen employed may vary widely and therefore may deviate from a preferred dosage regimen and one skilled in the art will recognize that dosages and dosage regimens outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4, part administrations. If appropriate, depending on individual behavior, it may be necessary to deviate upward or downward from the daily dose indicated.

The compounds and crystalline forms thereof, according to the present invention can be administrated in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may contain from 0.5 to about 90 percent of the active compound; however, an artisan of ordinary skill would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as solvents or suspending media. For this purpose any bland fixed oil may be employed including synthetic mono- or diglycerides. In addition, fatty acids such as oleic acid find use in the preparation of injectables.

The compounds and crystalline forms thereof, according to the present invention, may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present invention as an aerosol can be prepared by processes well-known to the person skilled in the art. For their preparation, for example, solutions or dispersions of the compounds of the present invention in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example, carbon dioxide, CFCs, such as, dichlorodifluoromethane, trichlorofluoromethane, and dichlorotetrafluoroethane, HFA's, such as, 1,1,1,2,3,3,3-heptafluororpropane and 1,1,1,2-tetrafluoroethane, and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethylcellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

Some embodiments of the present invention include a method of producing a pharmaceutical composition for “combination-therapy” comprising admixing at least one compound or crystalline form thereof as disclosed herein, together with at least one known pharmaceutical agent as described herein and a pharmaceutically acceptable carrier.

It is noted that when the H3-receptor modulators are utilized as active ingredients in a pharmaceutical composition, these are not intended for use only in humans, but in other non-human mammals as well. Indeed, recent advances in the area of animal health-care suggest that consideration be given for the use of active agents, such as H3-receptor modulators, for the treatment of an H3-receptor associated disease or disorder in domestic animals (e.g., cats and dogs) and in other domestic animals (e.g., such as cows, chickens, fish, etc). Those of ordinary skill in the art are readily credited with understanding the utility of such compounds in such settings.

### Indications and Methods

Histamine [2-(imidazol-4-yl)ethylamine] exerts its physiological effects through four distinct G-protein coupled receptors (GPCRs), termed H1, H2, H3 and H4. The histamine H3-receptor was first identified in 1983, when it was determined that the H3-receptor acted as an autoreceptor controlling both the synthesis and release of histamine (see: Arrang et al. Nature 1983, 302, 832-7). At least four human and three rat splice variants have proven functional activity in pharmacological assays (Passani et al., Trends in Pharmacol. Sci. 2004, 25, 618-625). Rat and human histamine H3-receptors also show constitutive activity which means that they can transduce a signal even in the absence of a ligand. Histamine H3-receptors also function as heteroceptors, modulating the release of a number of other transmitter substances including serotonin, acetylcholine, dopamine and noradrenaline (see: Brown et al. Prog. Neurobiol. 2001, 63, 637-672). Thus, there are a number of therapeutic applications for ligands that target the histamine H3-receptor, where the ligand functions as either an antagonist or inverse agonist (for reviews see: Leurs et al. Nat. Rev. Drug. Discov. 2005, 4, 107-120; Passani et al. Trends Pharmacol. Sci. 2004, 25, 618-625).

Accordingly, preclinical studies have identified a number of indications which are amenable to treatment with histamine H3-receptor antagonists and inverse agonists, such as compounds of the present invention. The compounds disclosed herein are believed to be useful in the treatment and/or prevention of several diseases and disorders, and in the amelioration of symptoms thereof. These compounds can be used alone or in combination with other compounds for the treatment and/or prevention of diseases and disorders. Without limitation, these diseases and disorders include the following.

Histamine H3-receptor antagonists have been shown to increase wakefulness (e.g. Lin J. S. et al. Brain Research 1990, 523, 325-330). This effect demonstrates that H3-receptor antagonists can be useful for treating disorders of sleep and wakefulness (Parmentier et al. J Neurosci. 2002, 22, 7695-7711; Ligneau et al. J. Pharmacol. Exp. Ther. 1998, 287, 658-666). For example, histamine H3-receptor antagonists and inverse agonists can be used to treat the somnolence syndrome associated with different pathological conditions, such as, sleep apnea and Parkinson's disease or circumstances associated with lifestyle, such as, daytime somnolence from sleep deprivation as a result of nocturnal jobs, overwork, or jet-lag (see Passani et al., Trends Pharmacol. Sci. 2004, 25, 618-625). Somnolence is a major public health problem because of its high prevalence (19-37% of the general population) and risk for causing work and traffic accidents.

Sleep apnea (alternatively sleep apnoea) is a common sleep disorder characterized by brief interruptions of breathing during sleep. These episodes, called apneas, last 10 seconds or more and occur repeatedly throughout the night. People with sleep apnea partially awaken as they struggle to breathe, but in the morning they may not be aware of the disturbances in their sleep. The most common type of sleep apnea is obstructive sleep apnea (OSA), caused by relaxation of soft tissue in the back of the throat that blocks the passage of air. Central sleep apnea (CSA) is caused by irregularities in the brain's normal signals to breathe. The hallmark symptom of the disorder is excessive daytime sleepiness. Additional symptoms of sleep apnea include restless sleep, loud snoring (with periods of silence followed by gasps), falling asleep during the day, morning headaches, trouble concentrating, irritability, forgetfulness, mood or behaviour changes, weight gain, increased heart rate, anxiety, and depression.

Few drug-based treatments of obstructive sleep apnea are known despite over two decades of research and tests. Oral administration of the methylxanthine theophylline (chemically similar to caffeine) can reduce the number of episodes of apnea, but can also produce side effects such as palpitations and insomnia. Theophylline is generally ineffective in adults with OSA, but is sometimes used to treat CSA, and infants and children with apnea. In 2003 and 2004, some neuroactive drugs, particularly modern-generation antidepressants including mirtazapine, have been reported to reduce incidences of obstructive sleep apnea. When other treatments do not completely treat the OSA, drugs are sometimes prescribed to treat a patient's daytime sleepiness or somnolence. These range from stimulants such as amphetamines to modern anti-narcoleptic medicines. The drug modafinil is seeing increased use in this role as of 2004.

In addition, for example, histamine H3-receptor antagonists and inverse agonists can be used to treat narcolepsy (Tedford et al. Soc. Neurosci. Abstr. 1999, 25, 460.3). Narcolepsy is a neurological condition most often characterized by Excessive Daytime Sleepiness (EDS), episodes of sleep and disorder of REM or rapid eye movement sleep. The main characteristic of narcolepsy is overwhelming Excessive Daytime Sleepiness (EDS), even after adequate nighttime sleep. A person with narcolepsy is likely to become drowsy or to fall asleep, often at inappropriate times and places. In addition, nighttime sleep may be fragmented with frequent wakenings. Classic symptoms of narcolepsy include, for example, cataplexy which is sudden episodes of loss of muscle function, ranging from slight weakness (such as limpness at the neck or knees, sagging facial muscles, or inability to speak clearly) to complete body collapse. Episodes may be triggered by sudden emotional reactions such as laughter, anger, surprise, or fear, and may last from a few seconds to several minutes. Another symptom of narcolepsy is sleep paralysis, which is the temporary inability to talk or move when waking up. Other symptoms include, for example, hypnagogic hallucinations which are vivid, often frightening, dream-like experiences that occur while dozing, falling asleep and/or while awakening, and automatic behaviour which occurs when a person continues to function (talking, putting things away, etc.) during sleep episodes, but awakens with no memory of performing such activities.

Daytime sleepiness, sleep paralysis, and hypnagogic hallucinations also occur in people who do not have narcolepsy, such as in people who are suffering from extreme lack of sleep. Cataplexy is generally considered unique to narcolepsy.

Currently the treatments available for narcolepsy treat the symptoms, but not the underlying cause. For cataplexy and REM-sleep symptoms, antidepressant medications and other drugs that suppress REM sleep are prescribed. The drowsiness is normally treated using stimulants such as methylphenidate (Ritalin), amphetamines (Adderall), dextroamphetamine (Dexedrine), methamphetamine (Desoxyn), modafinil (Provigil), etc. Other medications used are codeine and selegiline. The cataplexy is treated using clomipramine, imipramine, or protriptyline but this need only be done in severe cases. The drug gamma-hydroxybutyrate (GHB) (Xyrem) is approved in the USA by the Food and Drug Administration to treat both the cataplexy and excessive daytime sleepiness associated with narcolepsy.

Interestingly, modafinil (Provigil) has recently been shown to increase hypothalamic histamine release (Ishizuka et al. Neurosci. Lett. 2003, 339, 143-146).

In addition, recent studies using the classic Doberman model of narcolepsy with a non-imidazole histamine H3-receptor antagonist showed that a histamine H3-receptor antagonist can reduce the number of cataplectic attacks and the duration of the attacks (Carruthers Ann. Meet. Eur. Histamine Res. Soc. 2004, Abs. p 31).

In summary, histamine H3-receptor antagonists and inverse agonists can be used for the treatment and/or prevention of conditions associated with excessive daytime sleepiness such as hypersomnia, narcolepsy, sleep apnea, time zone change disorder, and other disorders which are associated with excessive daytime sleepiness such as fibromyalgia, and multiple sclerosis (Parmentier et al., J. Neurosci. 2002, 22, 7695-7711; Ligneau et al. J. Pharmacol. Exp. Ther. 1998; 287, 658-666). Other conditions include excessive sleepiness due to shift work, medical disorders, psychiatric disorders, narcolepsy, primary hypersomnia, and the like. Histamine H3-receptor antagonists and inverse agonists can also be used occasionally to promote wakefulness or vigilance in shift workers, sleep deprivation, post anesthesia grogginess, drowiness as a side effect from a medication, military use and the like.

In addition, wakefulness is a prerequisite for several brain functions including attention, learning, and memory and is required for appropriate behaviours in response to environmental challenges. Histamine H3-receptor antagonists and inverse agonists have been shown to improve cognitive performance in various animal models (Hancock and Fox in Milestones in Drug Therapy, ed. Buccafusco, 2003). These compounds can be used as pro-cognitive agents and can increase vigilance. Therefore, histamine H3-receptor antagonists and inverse agonists can be used in aging or degenerative disorders in which vigilance, attention and memory are impaired, for example, as in Alzheimer's disease or other dementias.

Alzheimer's disease (AD), a neurodegenerative disorder, is the most common cause of dementia. It is characterized clinically by progressive cognitive deterioration together with neuropsychiatric symptoms and behavioural changes. The most striking early symptom is memory loss, which usually manifests as minor forgetfulness that becomes steadily more pronounced with illness progression, with relative preservation of older memories. As the disorder progresses, cognitive (intellectual) impairment extends to the domains of language, skilled movements, recognition and functions closely related to the frontal and temporal lobes of the brain such as decision-making and planning. There is currently no cure for AD, although there are drugs which offer symptomatic benefit, specifically with respect to short-term memory impairment. These drugs include acetylcholinesterase inhibitors such as donepezil (Aricept), galantamine (Razadyne) and rivastigmine (Exelon) and NMDA antagonists such as memantine.

Histamine H3-receptor antagonists and inverse agonists can be used to treat or prevent cognitive disorders (Passani et al. Trends Pharmacol. Sci. 2004, 25, 618-625), epilepsy (Vohora et al. Pharmacol. Biochem. Behav. 2001, 68, 735-741), depression (Perez-Garcia et al. Psychopharmacol. 1999, 142, 215-220), attention deficit hyperactivity disorder (ADHD), (Fox et al. Behav. Brain Res. 2002, 131, 151-61), and schizophrenia (Fox et al. J. Pharmacol. Exp. Ther. 2005, 313, 176-190). These indications are described briefly below. For additional information, see reviews by Leurs et al., Nat. Rev. Drug. Discov. 2005, 4, 107-120, and Vohora Investigational Drugs 2004, 7, 667-673). Histamine H3-receptor antagonists or inverse agonists can also be used as a novel therapeutic approach to restore cortical activation in comatose or brain-traumatized patients (Passani et al., Trends in Pharmacol. Sci. 2004, 25, 618-625).

As stated above, histamine H3-receptor antagonists and inverse agonists can be used to treat or prevent epilepsy. Epilepsy (often referred to as a seizure disorder) is a chronic neurological condition characterized by recurrent unprovoked seizures. In terms of their pattern of activity, seizures may be described as either partial (focal) or generalized. Partial seizures only involve a localized part of the brain, whereas generalized seizures involve the entire cortex. There are many different epilepsy syndromes, each presenting with its own unique combination of seizure type, typical age of onset, EEG findings, treatment, and prognosis. Some common seizure syndromes include, for example, infantile spasms (West syndrome), childhood absence epilepsy, and benign focal epilepsy of childhood (Benign Rolandic epilepsy), juvenile myoclonic epilepsy, temporal lobe epilepsy, frontal lobe epilepsy and Lennox-Gastaut syndrome.

Compounds of the present invention can be used in combination with various known drugs. For example, compounds of the present invention can be used with one or more drugs that prevent seizures or reduce seizure frequency: these include carbamazepine (common brand name Tegretol), clobazam (Frisium), clonazepam (Klonopin), ethosuximide (Zarontin), felbamate (Felbatol), fosphenyloin (Cerebyx), flurazepam (Dalmane), gabapentin (Neurontin), lamotrigine (Lamictal), levetiracetam (Keppra), oxcarbazepine (Trileptal), mephenyloin (Mesantoin), phenobarbital (Luminal), phenyloin (Dilantin), pregabalin (Lyrica), primidone (Mysoline), sodium valproate (Epilim), tiagabine (Gabitril), topiramate (Topamax), valproate semisodium (Depakote), valproic acid (Depakene, Convulex), and vigabatrin (Sabril). Other drugs are commonly used to abort an active seizure or interrupt a seizure flurry; these include diazepam (Valium) and lorazepam (Ativan). Drugs used only in the treatment of refractory status epilepticus include paraldehyde (Paral) and pentobarbital (Nembutal).

As stated above, a histamine H3-receptor antagonist or inverse agonist can be used as the sole agent of treatment or can be used in combination with other agents. For example, Vohora et al. show that a histamine H3-receptor antagonist can work as an anti-epilepsy, anti-seizure drug and also showed effect with sub-effective doses of the H3-receptor antagonist in combination with sub-effective doses of known anti-epileptic drugs (Vohora et al. Pharmacol. Biochem. Behav. 2001, 68, 735-741).

Perez-Garcia et al. (Psychopharmacol. 1999, 142, 215-220) tested the ability of a histamine H3-receptor agonist and antagonist on experimental mouse models of anxiety (elevated plus-maze) and depression (forced swimming test). They found that while the compounds did not have a significant effect on the model of anxiety, a H3-receptor antagonist did have a significant dose-dependent effect in the model of depression. Thus, histamine H3-receptor antagonists or inverse agonists can have antidepressant effects.

Clinical depression is a state of sadness or melancholia that has advanced to the point of being disruptive to an individual's social functioning and/or activities of daily living. Clinical depression affects about 16% of the population on at least one occasion in their lives. Clinical depression is currently the leading cause of disability in the U.S. as well as other countries, and is expected to become the second leading cause of disability worldwide (after heart disease) by the year 2020, according to the World Health Organization.

Compounds of the present invention can be used in combination with various known drugs. For examples, compounds of the present invention can be used with one or more of the drugs currently available that can relieve the symptoms of depression. They include, for example, monoamine oxidase inhibitors (MAOIs) such as Nardil or Moclobemide (Manerix), tricyclic antidepressants, selective serotonin reuptake inhibitors (SSRIs) such as fluoxetine (Prozac), paroxetine (Paxil), escitalopram (Lexapro), and sertraline (Zoloft), norepinephrine reuptake inhibitors such as reboxetine (Edronax), and serotonin-norepinephrine reuptake inhibitors (SNRIs) such as venlafaxine (Effexor) and duloxetine (Cymbalta).

As stated above, histamine H3-receptor antagonists and inverse agonists can be used to treat or prevent attention deficit hyperactivity disorder (ADHD). According to the Diagnostic and Statistical Manual of Mental Disorders-IV-TR, ADHD is a developmental disorder that arises in childhood, in most cases before the age of 7 years, is characterized by developmentally inappropriate levels of inattention and/or hyperactive-impulsive behavior, and results in impairment in one or more major life activities, such as family, peer, educational, occupational, social, or adaptive functioning. ADHD can also be diagnosed in adulthood.

The first-line medications used to treat ADHD are mostly stimulants, which work by stimulating the areas of the brain responsible for focus, attention, and impulse control. The use of stimulants to treat a syndrome often characterized by hyperactivity is sometimes referred to as a paradoxical effect, but there is no real paradox in that stimulants activate brain inhibitory and self-organizing mechanisms permitting the individual to have greater self-regulation. The stimulants used include, for example, methylphenidate (sold as Ritalin, Ritalin SR and Ritalin LA), Metadate, Metadate ER, Metadate CD, Concerta, Focalin, Focalin XR or Methylin. The stimulants also include, for example, amphetamines such dextroamphetamine, sold as Dexedrine, Dexedrine Spansules, Adderall, and Adderall XR, a trade name for a mixture of dextroamphetamine and laevoamphetamine salts, methamphetamine sold as Desoxyn, bupropion, a dopamine and norepinephrine reuptake inhibitor, marketed under the brand name Wellbutrin. A non-stimulant medication to treat ADHD is Atomoxetine (sold as Strattera) a norepinephrine reuptake inhibitor. Other drugs sometimes used for ADHD include, for example, benzphetamine (Didrex), Provigil/Alertec/modafinil and clonidine. Recently it has been reported that in a rat pup model for ADHD, a histamine H3-receptor antagonist was at least as effective as methylphenidate (Ritalin) (Hancock and Fox in Milestones in Drug Therapy, ed. Buccafusco, 2003). Compounds of the present invention can be used in combination with various known drugs. For examples, compounds of the present invention can be used with one or more of the drugs used to treat ADHD and related disorders.

As stated above, histamine H3-receptor antagonists and inverse agonists can be used to treat or prevent schizophrenia. Schizophrenia is a psychiatric diagnosis that describes a mental disorder characterized by impairments in the perception or expression of reality and by significant social or occupational dysfunction. A person experiencing untreated schizophrenia is typically characterized as demonstrating disorganized thinking, and as experiencing delusions or auditory hallucinations. Although the disorder is primarily thought to affect cognition, it can also contribute to chronic problems with behavior and emotion. Schizophrenia is often described in terms of “positive” and “negative” symptoms. Positive symptoms include delusions, auditory hallucinations and thought disorder, and are typically regarded as manifestations of psychosis. Negative symptoms are so named because they are considered to be the loss or absence of normal traits or abilities, and include features such as flat, blunted or constricted affect and emotion, poverty of speech and lack of motivation. Some models of schizophrenia include formal thought disorder and planning difficulties in a third group, a “disorganization syndrome.”

The first line pharmacological therapy for schizophrenia is usually the use of antipsychotic medication. Antipsychotic drugs are only thought to provide symptomatic relief from the positive symptoms of psychosis. The newer atypical antipsychotic medications (such as clozapine, risperidone, olanzapine, quetiapine, ziprasidone and aripiprazole) are usually preferred over older typical antipsychotic medications (such as chlorpromazine and haloperidol) due to their favorable side-effect profile. While the atypical antipsychotics are associated with less extra pyramidal side-effects and tardive dyskinesia than the conventional antipsychotics, some of the agents in this class (especially olanzapine and clozapine) appear to be associated with metabolic side effects such as weight gain, hyperglycemia and hypertriglyceridemia that must be considered when choosing appropriate pharmacotherapy.

Histamine H3-receptor antagonists or inverse agonists can be used to treat obesity (Hancock, Curr. Opin. Investig. Drugs 2003, 4, 1190-1197). The role of neuronal histamine in food intake has been established for many years and neuronal histamine release and/or signalling has been implicated in the anorectic actions of known mediators in the feeding cycle such as leptin, amylin and bombesin. In the brain, the H3-receptor is implicated in the regulation of histamine release in the hypothalamus. Moreover, in situ hybridization studies have revealed histamine H3-receptor mRNA expression in rat brown adipose tissue, indicating a role in the regulation of thermogenesis (Karlstedt et al., Mol. Cell. Neurosci. 2003, 24, 614-622). Furthermore, histamine H3-receptor antagonists have been investigated in various preclinical models of obesity and have shown to be effective in reducing food intake, reducing weight, and decreasing total body fat in mice (Hancock, et al. Eur. J. Pharmacol. 2004, 487, 183-197). The most common drugs used for the treatment of obesity are sibutramine (Meridia) and orlistat (Xenical), both of which have limited effectiveness and significant side effects. Therefore, novel anti-obesity agents, such as histamine H3-receptor antagonists or inverse agonists, are needed.

Histamine H3-receptor antagonists or inverse agonists can also be used to treat upper airway allergic responses (U.S. Pat. Nos. 5,217,986; 5,352,707 and 5,869,479) including allergic rhinitis and nasal congestion. Allergic rhinitis is a frequently occurring chronic disease that affects a large number of people. Recent analysis of histamine H3-receptor expression in the periphery by quantitative PCR revealed that H3-receptor mRNA is abundantly expressed in human nasal mucosa (Varty et al. Eur. J. Pharmacol. 2004, 484, 83-89). In addition, in a cat model of nasal decongestion, a combination of histamine H3-receptor antagonists with the H1 receptor antagonist chlorpheniramine resulted in significant nasal decongestion without the hypertensive effect seen with adrenergic agonists. (McLeod et al. Am. J. Rhinol. 1999, 13, 391-399). Thus, histamine H3-receptor antagonists or inverse agonists can be used alone or in combination with H1 receptor blockage for the treatment of allergic rhinitis and nasal congestion.

Histamine H3-receptor antagonists or inverse agonists have therapeutic potential for the treatment of pain (Medhurst et al. Biochemical Pharmacology (2007), 73(8), 1182-1194).

The compound (R)-1-{2-[4′-(3-methoxy-propane-1-sulfonyl)-biphenyl-4-yl]-ethyl}-2-methyl-pyrrolidine mono-citrate and crystalline forms thereof, (R)-1-{2-[4′-(3-methoxy-propane-1-sulfonyl)-biphenyl-4-yl]-ethyl}-2-methyl-pyrrolidine di-citrate and the crystalline form thereof, (R)-1-{2-[4′-(3-methoxy-propane-1-sulfonyl)-biphenyl-4-yl]-ethyl}-2-methyl-pyrrolidine maleate and the crystalline form thereof and (R)-1-{2-[4′-(3-methoxy-propane-1-sulfonyl)-biphenyl-4-yl]-ethyl}-2-methyl-pyrrolidine hydrochloride and the crystalline form thereof, have activity as histamine H3-receptor modulators. Accordingly, each can be used in methods of modulating the histamine H3-receptor by contacting the receptor.

### (R)-1-{2-[4′-(3-Methoxy-propane-1-sulfonyl)-biphenyl-4-yl]-ethyl}-2-methyl-pyrrolidine mono-citrate and the crystalline forms thereof

In further embodiments, (R)-1-{2-[4′-(3-methoxy-propane-1-sulfonyl)-biphenyl-4-yl]-ethyl}-2-methyl-pyrrolidine mono-citrate and crystalline forms thereof can be used in methods for treating a histamine H3-receptor associated disorder in an individual comprising administering to the individual in need thereof a therapeutically effective amount of (R)-1-{2-[4′-(3-methoxy-propane-1-sulfonyl)-biphenyl-4-yl]-ethyl}-2-methyl-pyrrolidine mono-citrate or crystalline forms thereof or a pharmaceutical composition thereof.

The present invention further provides methods of treating diseases associated with the histamine H3-receptor in an individual (e.g., patient) by administering to the individual in need of such treatment a therapeutically effective amount or dose of (R)-1-{2-[4′-(3-methoxy-propane-1-sulfonyl)-biphenyl-4-yl]-ethyl}-2-methyl-pyrrolidine mono-citrate (including crystalline forms thereof) or a pharmaceutical composition thereof. Example diseases can include any disease, disorder or condition that is directly or indirectly linked to expression or activity of the histamine H3-receptor.

In some embodiments, the histamine H3-receptor associated disorder is a cognitive disorder, epilepsy, brain trauma, depression, obesity, a disorder of sleep and wakefulness, narcolepsy, cataplexy, hypersomnia, somnolence syndrome, jet lag, sleep apnea and the like, attention deficit hyperactivity disorder (ADHD), schizophrenia, allergies, an allergic response in the upper airway, allergic rhinitis, nasal congestion, dementia, Alzheimer's disease, and the like. In some embodiments, the histamine H3-receptor associated disorder is a disorder of sleep or wakefulness. In some embodiments, the histamine H3-receptor associated disorder is a cognitive disorder. In some embodiments, the histamine H3-receptor associated disorder is cataplexy.

The present invention further provides methods of inducing wakefulness in an individual comprising administering to said individual in need thereof a therapeutically effective amount of (R)-1-{2-[4′-(3-methoxy-propane-1-sulfonyl)-biphenyl-4-yl]-ethyl}-2-methyl-pyrrolidine mono-citrate (including crystalline forms thereof) or a pharmaceutical composition thereof.

One aspect of the present invention pertains to the use of (R)-1-{2-[4′-(3-methoxy-propane-1-sulfonyl)-biphenyl-4-yl]-ethyl}-2-methyl-pyrrolidine mono-citrate (including crystalline forms thereof) for the manufacture of a medicament for treating a histamine H3-receptor associated disorder.

One aspect of the present invention pertains to the use of (R)-1-{2-[4′-(3-methoxy-propane-1-sulfonyl)-biphenyl-4-yl]-ethyl}-2-methyl-pyrrolidine mono-citrate (including crystalline forms thereof) for the manufacture of a medicament for treating a histamine H3-receptor associated disorder selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, pain, dementia and Alzheimer's disease. In some embodiments, the histamine H3-receptor associated disorder is a disorder of sleep or wakefulness. In some embodiments, the histamine H3-receptor associated disorder is a cognitive disorder. In some embodiments, the histamine H3-receptor associated disorder is cataplexy.

One aspect of the present invention pertains to the use of (R)-1-{2-[4′-(3-methoxy-propane-1-sulfonyl)-biphenyl-4-yl]-ethyl}-2-methyl-pyrrolidine mono-citrate (including crystalline forms thereof) for the manufacture of a medicament for inducing wakefulness.

One aspect of the present invention pertains to the use of (R)-1-{2-[4′-(3-methoxy-propane-1-sulfonyl)-biphenyl-4-yl]-ethyl}-2-methyl-pyrrolidine mono-citrate (including crystalline forms thereof) for use in a method of treatment of the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine mono-citrate (including crystalline forms thereof) for use in a method for the treatment of a histamine H3-receptor associated disorder in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine mono-citrate (including crystalline forms thereof) for use in a method for the treatment of a histamine H3-receptor associated disorder selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea, attention deficit hyperactivity disorder (ADHD), schizophrenia, allergies, allergic responses in the upper airway, allergic rhinitis, nasal congestion, pain, dementia and Alzheimer's disease in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine mono-citrate (including crystalline forms thereof) for use in a method for the treatment of a disorder of sleep or wakefulness in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine mono-citrate (including crystalline forms thereof) for use in a method for the treatment of a cognitive disorder in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine mono-citrate (including crystalline forms thereof) for use in a method for the treatment of cataplexy in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine mono-citrate (including crystalline forms thereof) for use in a method of inducing wakefulness in the human or animal body by therapy.

### (R)-1-{2-[4′-(3-Methoxy-propane-1-sulfonyl)-biphenyl-4-yl]-ethyl}-2-methyl-pyrrolidine di-citrate and the crystalline form thereof

In further embodiments, (R)-1-{2-[4′-(3-methoxy-propane-1-sulfonyl)-biphenyl-4-yl]-ethyl}-2-methyl-pyrrolidine di-citrate and the crystalline form thereof can be used in methods for treating a histamine H3-receptor associated disorder in an individual comprising administering to the individual in need thereof a therapeutically effective amount of (R)-1-{2-[4′-(3-methoxy-propane-1-sulfonyl)-biphenyl-4-yl]-ethyl}-2-methyl-pyrrolidine di-citrate (including the crystalline form thereof) or a pharmaceutical composition thereof.

The present invention further provides methods of treating diseases associated with the histamine H3-receptor in an individual (e.g., patient) by administering to the individual in need of such treatment a therapeutically effective amount or dose of (R)-1-{2-[4′-(3-methoxy-propane-1-sulfonyl)-biphenyl-4-yl]-ethyl}-2-methyl-pyrrolidine di-citrate (including the crystalline form thereof) or a pharmaceutical composition thereof. Example diseases can include any disease, disorder or condition that is directly or indirectly linked to expression or activity of the histamine H3-receptor.

In some embodiments, the histamine H3-receptor associated disorder is a cognitive disorder, epilepsy, brain trauma, depression, obesity, disorders of sleep and wakefulness, narcolepsy, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, dementia, Alzheimer's disease, and the like. In some embodiments, the histamine H3-receptor associated disorder is a disorder of sleep or wakefulness. In some embodiments, the histamine H3-receptor associated disorder is a cognitive disorder. In some embodiments, the histamine H3-receptor associated disorder is cataplexy.

The present invention further provides methods of inducing wakefulness in an individual comprising administering to said individual in need thereof a therapeutically effective amount of (R)-1-{2-[4′-(3-methoxy-propane-1-sulfonyl)-biphenyl-4-yl]-ethyl}-2-methyl-pyrrolidine di-citrate (including the crystalline form thereof) or a pharmaceutical composition thereof.

One aspect of the present invention pertains to the use of (R)-1-{2-[4′-(3-methoxy-propane-1-sulfonyl)-biphenyl-4-yl]-ethyl}-2-methyl-pyrrolidine di-citrate (including the crystalline form thereof) for the manufacture of a medicament for treating a histamine H3-receptor associated disorder.

One aspect of the present invention pertains to the use of (R)-1-{2-[4′-(3-methoxy-propane-1-sulfonyl)-biphenyl-4-yl]-ethyl}-2-methyl-pyrrolidine di-citrate (including the crystalline form thereof) for the manufacture of a medicament for treating a histamine H3-receptor associated disorder selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea and the like, attention deficit hyperactivity disorder (ADM), schizophrenia, allergies, allergic responses in the upper airway, allergic rhinitis, nasal congestion, pain, dementia and Alzheimer's disease. In some embodiments, the histamine H3-receptor associated disorder is a disorder of sleep or wakefulness. In some embodiments, the histamine H3-receptor associated disorder is a cognitive disorder. In some embodiments, the histamine H3-receptor associated disorder is cataplexy.

One aspect of the present invention pertains to the use of (R)-1-{2-[4′-(3-methoxy-propane-1-sulfonyl)-biphenyl-4-yl]-ethyl}-2-methyl-pyrrolidine di-citrate (including the crystalline form thereof) for the manufacture of a medicament for inducing wakefulness.

One aspect of the present invention pertains to the use of (R)-1-{2-[4′-(3-methoxy-propane-1-sulfonyl)-biphenyl-4-yl]-ethyl}-2-methyl-pyrrolidine di-citrate (including the crystalline form thereof) for use in a method of treatment of the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine di-citrate (including the crystalline form thereof) for use in a method for the treatment of a histamine H3-receptor associated disorder in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine di-citrate (including the crystalline form thereof) for use in a method for the treatment of a histamine H3-receptor associated disorder selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea, attention deficit hyperactivity disorder (ADHD), schizophrenia, allergies, allergic responses in the upper airway, allergic rhinitis, nasal congestion, pain, dementia and Alzheimer's disease in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine di-citrate (including the crystalline form thereof) for use in a method for the treatment of a disorder of sleep or wakefulness in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine di-citrate (including the crystalline form thereof) for use in a method for the treatment of a cognitive disorder in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine di-citrate (including the crystalline form thereof) for use in a method for the treatment of cataplexy in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine di-citrate (including the crystalline form thereof) for use in a method of inducing wakefulness in the human or animal body by therapy.

### (R)-1-{2-[4′-(3-Methoxy-propane-1-sulfonyl)-biphenyl-4-yl]-ethyl}-2-methyl-pyrrolidine maleate and the crystalline form thereof

In further embodiments, (R)-1-{2-[4′-(3-methoxy-propane-1-sulfonyl)-biphenyl-4-yl]-ethyl}-2-methyl-pyrrolidine maleate and the crystalline form thereof can be used in methods for treating a histamine H3-receptor associated disorder in an individual comprising administering to the individual in need thereof a therapeutically effective amount of (R)-1-{2-[4′-(3-methoxy-propane-1-sulfonyl)-biphenyl-4-yl]-ethyl}-2-methyl-pyrrolidine maleate or the crystalline form thereof or a pharmaceutical composition thereof.

The present invention further provides methods of treating diseases associated with the histamine H3-receptor in an individual (e.g., patient) by administering to the individual in need of such treatment a therapeutically effective amount or dose of (R)-1-{2-[4′-(3-methoxy-propane-1-sulfonyl)-biphenyl-4-yl]-ethyl}-2-methyl-pyrrolidine maleate (including the crystalline form thereof) or a pharmaceutical composition thereof. Example diseases can include any disease, disorder or condition that is directly or indirectly linked to expression or activity of the histamine H3-receptor.

In some embodiments, the histamine H3-receptor associated disorder is a cognitive disorder, epilepsy, brain trauma, depression, obesity, a disorder of sleep and wakefulness, narcolepsy, cataplexy, hypersomnia, somnolence syndrome, jet lag, sleep apnea and the like, attention deficit hyperactivity disorder (ADHD), schizophrenia, allergies, an allergic response in the upper airway, allergic rhinitis, nasal congestion, dementia, Alzheimer's disease, and the like. In some embodiments, the histamine H3-receptor associated disorder is a disorder of sleep or wakefulness. In some embodiments, the histamine H3-receptor associated disorder is a cognitive disorder. In some embodiments, the histamine H3-receptor associated disorder is cataplexy.

The present invention further provides methods of inducing wakefulness in an individual comprising administering to said individual in need thereof a therapeutically effective amount of (R)-1-{2-[4′-(3-methoxy-propane-1-sulfonyl)-biphenyl-4-yl]-ethyl}-2-methyl-pyrrolidine maleate (including the crystalline form thereof) or a pharmaceutical composition thereof.

One aspect of the present invention pertains to the use of (R)-1-{2-[4′-(3-methoxy-propane-1-sulfonyl)-biphenyl-4-yl]-ethyl}-2-methyl-pyrrolidine maleate (including the crystalline form thereof) for the manufacture of a medicament for treating a histamine H3-receptor associated disorder.

One aspect of the present invention pertains to the use of (R)-1-{2-[4′-(3-methoxy-propane-1-sulfonyl)-biphenyl-4-yl]-ethyl}-2-methyl-pyrrolidine maleate (including the crystalline form thereof) for the manufacture of a medicament for treating a histamine H3-receptor associated disorder selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, pain, dementia and Alzheimer's disease. In some embodiments, the histamine H3-receptor associated disorder is a disorder of sleep or wakefulness. In some embodiments, the histamine H3-receptor associated disorder is a cognitive disorder. In some embodiments, the histamine H3-receptor associated disorder is cataplexy.

One aspect of the present invention pertains to the use of (R)-1-{2-[4′-(3-methoxy-propane-1-sulfonyl)-biphenyl-4-yl]-ethyl}-2-methyl-pyrrolidine maleate (including the crystalline form thereof) for the manufacture of a medicament for inducing wakefulness.

One aspect of the present invention pertains to the use of (R)-1-{2-[4′-(3-methoxy-propane-1-sulfonyl)-biphenyl-4-yl]-ethyl}-2-methyl-pyrrolidine maleate (including the crystalline form thereof) for use in a method of treatment of the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine maleate (including the crystalline form thereof) for use in a method for the treatment of a histamine H3-receptor associated disorder in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine maleate (including the crystalline form thereof) for use in a method for the treatment of a histamine H3-receptor associated disorder selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea, attention deficit hyperactivity disorder (ADHD), schizophrenia, allergies, allergic responses in the upper airway, allergic rhinitis, nasal congestion, pain, dementia and Alzheimer's disease in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine maleate (including the crystalline form thereof) for use in a method for the treatment of a disorder of sleep or wakefulness in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine maleate (including the crystalline form thereof) for use in a method for the treatment of a cognitive disorder in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine maleate (including the crystalline form thereof) for use in a method for the treatment of cataplexy in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine maleate (including the crystalline form thereof) for use in a method of inducing wakefulness in the human or animal body by therapy.

### (R)-1-{2-[4′-(3-Methoxy-propane-1-sulfonyl)-biphenyl-4-yl]-ethyl}-2-methyl-pyrrolidine hydrochloride and the crystalline form thereof

In further embodiments, (R)-1-{2-[4′-(3-methoxy-propane-1-sulfonyl)-biphenyl-4-yl]-ethyl}-2-methyl-pyrrolidine hydrochloride and the crystalline form thereof can be used in methods for treating a histamine H3-receptor associated disorder in an individual comprising administering to the individual in need thereof a therapeutically effective amount of (R)-1-{2-[4′-(3-methoxy-propane-1-sulfonyl)-biphenyl-4-yl]-ethyl}-2-methyl-pyrrolidine hydrochloride or the crystalline form thereof or a pharmaceutical composition thereof.

The present invention further provides methods of treating diseases associated with the histamine H3-receptor in an individual (e.g., patient) by administering to the individual in need of such treatment a therapeutically effective amount or dose of (R)-1-{2-[4′-(3-methoxy-propane-1-sulfonyl)-biphenyl-4-yl]-ethyl}-2-methyl-pyrrolidine hydrochloride (including the crystalline form thereof) or a pharmaceutical composition thereof. Example diseases can include any disease, disorder or condition that is directly or indirectly linked to expression or activity of the histamine H3-receptor.

In some embodiments, the histamine H3-receptor associated disorder is a cognitive disorder, epilepsy, brain trauma, depression, obesity, a disorder of sleep and wakefulness, narcolepsy, cataplexy, hypersomnia, somnolence syndrome, jet lag, sleep apnea and the like, attention deficit hyperactivity disorder (ADHD), schizophrenia, allergies, an allergic response in the upper airway, allergic rhinitis, nasal congestion, dementia, Alzheimer's disease, and the like. In some embodiments, the histamine H3-receptor associated disorder is a disorder of sleep or wakefulness. In some embodiments, the histamine H3-receptor associated disorder is a cognitive disorder. In some embodiments, the histamine H3-receptor associated disorder is cataplexy.

The present invention further provides methods of inducing wakefulness in an individual comprising administering to said individual in need thereof a therapeutically effective amount of (R)-1-{2-[4′-(3-methoxy-propane-1-sulfonyl)-biphenyl-4-yl]-ethyl}-2-methyl-pyrrolidine hydrochloride (including the crystalline form thereof) or a pharmaceutical composition thereof.

One aspect of the present invention pertains to the use of (R)-1-{2-[4′-(3-methoxy-propane-1-sulfonyl)-biphenyl-4-yl]-ethyl}-2-methyl-pyrrolidine hydrochloride (including the crystalline form thereof) for the manufacture of a medicament for treating a histamine H3-receptor associated disorder.

One aspect of the present invention pertains to the use of (R)-1-{2-[4′-(3-methoxy-propane-1-sulfonyl)-biphenyl-4-yl]-ethyl}-2-methyl-pyrrolidine hydrochloride (including the crystalline form thereof) for the manufacture of a medicament for treating a histamine H3-receptor associated disorder selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, pain, dementia and Alzheimer's disease. In some embodiments, the histamine H3-receptor associated disorder is a disorder of sleep or wakefulness. In some embodiments, the histamine H3-receptor associated disorder is a cognitive disorder. In some embodiments, the histamine H3-receptor associated disorder is cataplexy.

One aspect of the present invention pertains to the use of (R)-1-{2-[4′-(3-methoxy-propane-1-sulfonyl)-biphenyl-4-yl]-ethyl}-2-methyl-pyrrolidine hydrochloride (including the crystalline form thereof) for the manufacture of a medicament for inducing wakefulness.

One aspect of the present invention pertains to the use of (R)-1-{2-[4′-(3-methoxy-propane-1-sulfonyl)-biphenyl-4-yl]-ethyl}-2-methyl-pyrrolidine hydrochloride (including the crystalline form thereof) for use in a method of treatment of the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine hydrochloride (including the crystalline form thereof) for use in a method for the treatment of a histamine H3-receptor associated disorder in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine hydrochloride (including the crystalline form thereof) for use in a method for the treatment of a histamine H3-receptor associated disorder selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea, attention deficit hyperactivity disorder (ADHD), schizophrenia, allergies, allergic responses in the upper airway, allergic rhinitis, nasal congestion, pain, dementia and Alzheimer's disease in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine hydrochloride (including the crystalline form thereof) for use in a method for the treatment of a disorder of sleep or wakefulness in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine hydrochloride (including the crystalline form thereof) for use in a method for the treatment of a cognitive disorder in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine hydrochloride (including the crystalline form thereof) for use in a method for the treatment of cataplexy in the human or animal body by therapy.

One aspect of the present invention pertains to the use of (R)-1-{2-[4′-(3-methoxy-propane-1-sulfonyl)-biphenyl-4-yl]-ethyl}-2-methyl-pyrrolidine hydrochloride (including the crystalline form thereof) for use in a method of inducing wakefulness in the human or animal body by therapy.

### Preparation of Crystalline Forms of Mono-Citrate, Di-Citrate, Maleate and Hydrochloride of (R)-1-{2-[4′-(3-Methoxy-propane-1-sulfonyl)-biphenyl-4-yl]-ethyl}-2-methyl-pyrrolidine

### Processes for Preparing the crystalline form of (R)-1-{2-[4′-(3-methoxy-propane-1-sulfonyl)-biphenyl-4-yl]-ethyl}-2-methyl-pyrrolidine mono-citrate (FORM 1)

One aspect of the present invention is directed to processes for preparing (R)-1-{2-[4′-(3-methoxy-propane-1-sulfonyl)-biphenyl-4-yl]-ethyl}-2-methyl-pyrrolidine mono-citrate comprising the steps of:
1) combining (R)-1-{2-[4′-(3-methoxy-propane-1-sulfonyl)-biphenyl-4-yl]-ethyl}-2-methyl-pyrrolidine and a suitable organic solvent to form a solution;
2) adding an aqueous solution comprising citric acid to the solution of (R)-1-{2-[4′-(3-methoxy-propane-1-sulfonyl)-biphenyl-4-yl]-ethyl}-2-methyl-pyrrolidine to form a precipitate; and
3) separating the precipitate, wherein the precipitate is (R)-1-{2-[4′-(3-methoxy-propane-1-sulfonyl)-biphenyl-4-yl]-ethyl}-2-methyl-pyrrolidine mono-citrate.

In some embodiments, processes for preparing (R)-1-{2-[4′-(3-methoxy-propane-1-sulfonyl)-biphenyl-4-yl]-ethyl}-2-methyl-pyrrolidine mono-citrate further comprise the step of adding a seed crystal to the solution of (R)-1-{2-[4′-(3-methoxy-propane-1-sulfonyl)-biphenyl-4-yl]-ethyl}-2-methyl-pyrrolidine before separating the precipitate.

In some embodiments, the seed crystal is (R)-1-{2-[4′-(3-methoxy-propane-1-sulfonyl)-biphenyl-4-yl]-ethyl}-2-methyl-pyrrolidine mono-citrate.

In some embodiments, the seed crystal is (R)-1-{2-[4′-(3-methoxy-propane-1-sulfonyl)-biphenyl-4-yl]-ethyl}-2-methyl-pyrrolidine di-citrate.

In some embodiments, the molar ratio of citric acid present in the aqueous solution compared to (R)-1-{2-[4′-(3-methoxy-propane-1-sulfonyl)-biphenyl-4-yl]-ethyl}-2-methyl-pyrrolidine is about 1 to 1.

In some embodiments, the suitable organic solvent is acetonitrile.

### Processes for preparing the crystalline form of (R)-1-{2-[4′-(3-methoxy-propane-1-sulfonyl)-biphenyl-4-yl]-ethyl}-2-methyl-pyrrolidine mono-citrate (FORM 2)

One aspect of the present invention is directed to processes for preparing (R)-1-{2-[4′-(3-methoxy-propane-1-sulfonyl)-biphenyl-4-yl]-ethyl}-2-methyl-pyrrolidine mono-citrate comprising the steps of:
1) combining (R)-1-{2-[4′-(3-methoxy-propane-1-sulfonyl)-biphenyl-4-yl]-ethyl}-2-methyl-pyrrolidine and a suitable organic solvent to form a solution;
2) adding an aqueous solution comprising citric acid to the solution of (R)-1-{2-[4′-(3-methoxy-propane-1-sulfonyl)-biphenyl-4-yl]-ethyl}-2-methyl-pyrrolidine to form a mono-citrate solution; and
3) isolating the (R)-1-{2-[4′-(3-methoxy-propane-1-sulfonyl)-biphenyl-4-yl]-ethyl}-2-methyl-pyrrolidine mono-citrate from the mono-citrate solution.

In some embodiments, the molar ratio of citric acid present in the aqueous solution compared to (R)-1-{2-[4′-(3-methoxy-propane-1-sulfonyl)-biphenyl-4-yl]-ethyl}-2-methyl-pyrrolidine is about 1 to 1.

In some embodiments, the suitable organic solvent is a water-miscible organic solvent.

In some embodiments, the suitable organic solvent is acetone.

In some embodiments, the suitable organic solvent is isopropyl alcohol.

### Processes for preparing the crystalline form of (R)-1-{2-[4′-(3-methoxy-propane-1-sulfonyl)-biphenyl-4-yl]-ethyl}-2-methyl-pyrrolidine di-citrate

One aspect of the present invention is directed to processes for preparing (R)-1-{2-[4′-(3-methoxy-propane-1-sulfonyl)-biphenyl-4-yl]-ethyl}-2-methyl-pyrrolidine di-citrate comprising the steps of:
1) combining (R)-1-{2-[4′-(3-methoxy-propane-1-sulfonyl)-biphenyl-4-yl]-ethyl}-2-methyl-pyrrolidine and a suitable organic solvent to form a solution;
2) adding an aqueous solution comprising citric acid to the solution of (R)-1-{2-[4′-(3-methoxy-propane-1-sulfonyl)-biphenyl-4-yl]-ethyl}-2-methyl-pyrrolidine to form a precipitate; and
3) separating the precipitate, wherein the precipitate is (R)-1-{2-[4′-(3-methoxy-propane-1-sulfonyl)-biphenyl-4-yl]-ethyl}-2-methyl-pyrrolidine di-citrate.

In some embodiments, the molar ratio of citric acid present in the aqueous solution compared to (R)-1-{2-[4′-(3-methoxy-propane-1-sulfonyl)-biphenyl-4-yl]-ethyl}-2-methyl-pyrrolidine is at least 2 to 1.

In some embodiments, the molar ratio of citric acid present in the aqueous solution compared to (R)-1-{2-[4′-(3-methoxy-propane-1-sulfonyl)-biphenyl-4-yl]-ethyl}-2-methyl-pyrrolidine is about 2.1 to 1.

In some embodiments, the suitable organic solvent is acetonitrile.

In some embodiments, the aqueous solution comprising citric acid is at a temperature above about 25° C.

### Processes for preparing the crystalline form of (R)-1-{2-[4′-(3-Methoxy-propane-1-sulfonyl)-biphenyl-4-yl]-ethyl}-2-methyl-pyrrolidine maleate

One aspect of the present invention is directed to processes for preparing (R)-1-{2-[4′-(3-methoxy-propane-1-sulfonyl)-biphenyl-4-yl]-ethyl}-2-methyl-pyrrolidine maleate comprising the steps of:
1) combining (R)-1-{2-[4′-(3-methoxy-propane-1-sulfonyl)-biphenyl-4-yl]-ethyl}-2-methyl-pyrrolidine and a suitable organic solvent to form a solution;
2) adding an aqueous solution comprising maleic acid to the solution of (R)-1-{2-[4′-(3-methoxy-propane-1-sulfonyl)-biphenyl-4-yl]-ethyl}-2-methyl-pyrrolidine to form a maleate solution; and
3) isolating the (R)-1-{2-[4′-(3-methoxy-propane-1-sulfonyl)-biphenyl-4-yl]-ethyl}-2-methyl-pyrrolidine maleate from the maleate solution.

In some embodiments, the molar ratio of maleic acid present in the aqueous solution compared to (R)-1-{2-[4′-(3-methoxy-propane-1-sulfonyl)-biphenyl-4-yl]-ethyl}-2-methyl-pyrrolidine is about 1 to 1.

In some embodiments, processes for preparing (R)-1-{2-[4′-(3-methoxy-propane-1-sulfonyl)-biphenyl-4-yl]-ethyl}-2-methyl-pyrrolidine maleate further comprise the step of adding a seed crystal to the solution of (R)-1-{2-[4′-(3-methoxy-propane-1-sulfonyl)-biphenyl-4-yl]-ethyl}-2-methyl-pyrrolidine before isolating the (R)-1-{2-[4′-(3-methoxy-propane-1-sulfonyl)-biphenyl-4-yl]-ethyl}-2-methyl-pyrrolidine maleate.

In some embodiments, the seed crystal is (R)-1-{2-[4′-(3-methoxy-propane-1-sulfonyl)-biphenyl-4-yl]-ethyl}-2-methyl-pyrrolidine maleate.

In some embodiments, the suitable organic solvent is a water-miscible organic solvent.

In some embodiments, the suitable organic solvent is acetone.

In some embodiments, the suitable organic solvent is isopropyl alcohol.

### Processes for preparing the crystalline form of (R)-1-{2-[4′-(3-Methoxy-propane-1-sulfonyl)-biphenyl-4-yl]-ethyl}-2-methyl-pyrrolidine hydrochloride

One aspect of the present invention is directed to processes for preparing (R)-1-{2-[4′-(3-methoxy-propane-1-sulfonyl)-biphenyl-4-yl]-ethyl}-2-methyl-pyrrolidine hydrochloride comprising the steps of:
1) combining (R)-1-{2-[4′-(3-methoxy-propane-1-sulfonyl)-biphenyl-4-yl]-ethyl}-2-methyl-pyrrolidine and a suitable organic solvent to form a solution;
2) adding hydrochloric acid to said solution of (R)-1-{2-[4′-(3-methoxy-propane-1-sulfonyl)-biphenyl-4-yl]-ethyl}-2-methyl-pyrrolidine to form a precipitate; and
3) separating said precipitate, wherein the said precipitate is (R)-1-{2-[4′-(3-methoxy-propane-1-sulfonyl)-biphenyl-4-yl]-ethyl}-2-methyl-pyrrolidine hydrochloride.

In some embodiments, the suitable organic solvent is diethylether.

In some embodiments, hydrochloric acid is anhydrous.

In some embodiments, hydrochloric acid is dissolved in diethylether.

### DEFINITIONS

The term “contact or contacting” is intended to mean bringing the indicated moieties together, whether in an in vitro system or an in vivo system. Thus, “contacting” a histamine H3-receptor with a compound of the invention includes the administration of a compound of the present invention to an individual, preferably a human, having a histamine H3-receptor, as well as, for example, introducing a compound of the invention into a sample containing a cellular or more purified preparation containing a histamine H3-receptor.

The term “extrapolated onset temperature” is intended to mean the temperature determined at the intersection of the line tangent to the curve at the inflection point (determined by the software) with the extrapolated baseline before the peak temperature.

The term “hydrate” as used herein means a salt of the invention that further includes a stoichiometric or non-stoichiometric amount of water bound by non-covalent intermolecular forces.

The term “solvate” as used herein means a salt of the invention that further includes a stoichiometric or non-stoichiometric amount of a solvent bound by non-covalent intermolecular forces. Preferred solvents are volatile, non-toxic, and/or acceptable for administration to humans in trace amounts.

The term “in need of treatment” and the term “in need thereof” when referring to treatment are used interchangeably to mean a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

The term “individual” is intended to mean any animal, including mammals, preferably mice, rats, other rodents, rabbits, dogs, cats, swine, cattle, sheep, horses, or primates, and most preferably humans.

The term “modulate or modulating” is intended to mean an increase or decrease in the amount, quality, response or effect of a particular activity, function or molecule.

The term “pharmaceutical composition” is intended to mean a composition comprising at least one active ingredient; including but not limited to, salts, solvates and hydrates of compounds of the present invention; whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “precipitate” is intended to mean the formation of a solid or semi-solid that settles out of solution.

The term “therapeutically effective amount” is intended to mean the amount of active compound or pharmaceutical agent that elicits the biological or medicinal response in a tissue, system, animal, individual or human that is being sought by a researcher, veterinarian, medical doctor or other clinician, which includes one or more of the following:

(1) Preventing the disease; for example, preventing a disease, condition or disorder in an individual that may be predisposed to the disease, condition or disorder but does not yet experience or display the pathology or symptomatology of the disease,

(2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and

(3) Ameliorating the disease; for example, ameliorating a disease, condition or disorder in an individual that is experiencing or displaying the pathology or symptomatology of the disease, condition or disorder (i.e., reversing the pathology and/or symptomatology).

In order that the invention disclosed herein may be more efficiently understood, examples are provided below. It should be understood that these examples are for illustrative purposes only and are not to be construed as limiting the invention in any manner.

### EXAMPLES

### Example 1

### Syntheses of Compounds of the Present Invention

The compounds of the invention and their synthesis are further illustrated by the following examples. The following examples are provided to further define the invention without, however, limiting the invention to the particulars of these examples. The compounds described herein, supra and infra, are named according to the CS ChemDraw Ultra Version 7.0.1, AutoNom version 2.2 or CS ChemDraw Ultra Version 9.0.7. In certain instances common names are used and it is understood that these common names would be recognized by those skilled in the art.

Chemistry: Proton nuclear magnetic resonance (¹H NMR) spectra were recorded on a Varian Mercury Vx-400 equipped with a 4 nucleus auto switchable probe and z-gradient or a Bruker Avance-400 equipped with a QNP (Quad Nucleus Probe) or a BBI (Broad Band Inverse) and z-gradient. Chemical shifts are given in parts per million (ppm) with the residual solvent signal used as reference. NMR abbreviations are used as follows: s=singlet, d=doublet, t=triplet, q=quartet, m=multiplet, br=broad, dt=doublet of triplet, td=triplet of doublet, dd=doublet of doublet, ddd=doublet of doublet of doublets. Microwave irradiations were carried out using a Smith Synthesizer™ or an Emrys Optimizer™ (Personal Chemistry). Thin-layer chromatography (TLC) was performed on silica gel 60 F₂₅₄ (Merck), preparatory thin-layer chromatography (prep TLC) was preformed on PK6F silica gel 60 A 1 mm plates (Whatman), and column chromatography was carried out on a silica gel column using Kieselgel 60, 0.063-0.200 mm (Merck). Evaporation was done under reduced pressure on a Büchi rotary evaporator. Celite 545® was used during palladium filtrations.

LCMS specs: HPLC-pumps: LC-10AD VP, Shimadzu Inc.; HPLC system controller: SCL-10A VP, Shimadzu Inc; UV-Detector: SPD-10A VP, Shimadzu Inc; Autosampler: CTC HTS, PAL, Leap Scientific; Mass spectrometer: API 150EX with Turbo Ion Spray source, AB/MDS Sciex; Software: Analyst 1.2.

### Example 1.1

### Preparation of (R)-1-{2-[4′-(3-Methoxy-propane-1-sulfonyl)-biphenyl-4-yl]-ethyl}-2-methyl-pyrrolidine (Method 1)

### Step A: Preparation of Intermediate 4′-(2-Chloro-ethyl)-biphenyl-4-sulfonyl Chloride

To a nitrogen-purged reactor vented to an aqueous sodium hydroxide scrubber was added 4′-(2-chloro-ethyl)-biphenyl-4-sulfonic acid (2.101 kg after correction for 3.7 wt % water content, 7.08 mol) followed by thionyl chloride (5.358 L, 8.74 kg, 73.5 mol). The resulting mixture was stirred and cooled to −2.5° C. N,N-Dimethylacetamide (68 mL, 63.7 g, 0.731 mol) was then added sufficiently slowly to maintain the stirred reactor contents at −3 to 0° C. with reactor jacket cooling. The reactor contents were heated to 63° C., and stirring at 63-66° C. was continued for 6.4 h until conversion of the starting material to the product was verified to be substantially complete by LC/MS analysis. After the reactor contents had been cooled to 19° C., heptane (7.62 L) was added in six equal portions over 2.5 h. Volatiles consisting mostly of thionyl chloride were then distilled off the product mixture at 30-32° C. and pressures falling to 109 torr. The condensate volume was 4 L. The concentrated product mixture was cooled to 22° C., and stirring at 20-22° C. was continued for 15.4 h. The product mixture was then filtered. The filtered solid was washed with heptane (11 L) and then deionized water (11 L), both at ambient temperature, and then vacuum dried at 40° C. to constant weight to provide 4′-(2-chloro-ethyl)-biphenyl-4-sulfonyl chloride (1.664 kg, 74.6% yield, 97.9% purity by HPLC peak area).

### Step B: Preparation of Intermediate Sodium 4′-(2-Chloroethyl)-4-biphenylsulfinate

To a nitrogen-purged reactor containing deionized water (14.1 L) stirred at 29° C. was added sodium sulfite (3.3145 kg, 26.3 mol), Na₂HPO₄ (0.7459 kg, 5.25 mol) and benzyltriethylammonium chloride (65.1 g, 0.265 mol). Addition of the benzyltriethylammonium chloride caused the temperature of the reactor contents to rise to 37° C. All the reagents dissolved upon continued stirring at 35-37° C. for 16 min, after which 4′-(2-chloro-ethyl)-biphenyl-4-sulfonyl chloride (1.6584 g, 5.26 mol) and a deionized water rinse (2.5 L) were added. The temperature of the stirred reactor contents was then increased to 57° C., and stirring at 57-60° C. under nitrogen was continued for 6 h until LC/MS analysis revealed complete conversion of the starting material. The stirred mixture was cooled to 42° C. and then filtered. Deionized water (8.3 L) was added to the reactor and heated with stirring to 36° C. The filtered solids were then added back to the reactor, and the resulting mixture was stirred at 38° C. overnight before being filtered. The filtered solid was washed at ambient temperature first with deionized water (3.3 L) and then twice with acetonitrile (3.3 L and then 2.8 L). The washed solids were vacuum dried at 60° C. to provide crude white sodium 4′-(2-chloroethyl)-4-biphenylsulfinate (1.2282 kg, 77.1% yield, 92.6% pure by HPLC peak area) containing 4′-(2-chloro-ethyl)-biphenyl-4-sulfonic acid (7.4 HPLC area %).

### Step C: Preparation of 4-(2-Chloro-ethyl)-4′-(3-methoxy-propane-1-sulfonyl)-biphenyl and 4-(2-Bromo-ethyl)-4′-(3-methoxy-propane-1-sulfonyl)-biphenyl

To a stirred mixture of sodium 4′-(2-chloroethyl)-4-biphenylsulfinate (217.9 g, 719.7 mmol,), sodium phosphate, dibasic (102.2 g, 719.7 mmol), tetabutylammonium bromide (TBAB) (232.0 g, 719.7 mmol), potassium bromide (85.65 g, 719.7 mmol) and deionized water (809 mL) was added 1-bromo-3-methoxypropane (137.7 g, 899.9 mmol) at ambient temperature. The resulting mixture became a clear solution as it was stirred and heated under nitrogen to 80° C. After the reaction mixture had been stirred at 80° C. for 16 h, additional 1-bromo-3-methoxypropane (12.11 g, 79.1 mmol) was added. After another 4 h of stirring at 80° C., more 1-bromo-3-methoxypropane (6.0 g, 39.2 mmol) was added. Heating at 80° C. was continued for two more hours (for a total of 22 h) and then discontinued. Methanol (1.09 L) was added when the mixture had cooled to about 65° C., and the stirred mixture was then allowed to cool to ambient temperature overnight. The resulting white precipitate was filtered, slurry-washed with deionized water (2×500 mL), air-dried, and then stirred in ethyl acetate (1.0 L) for 1 h at ambient temperature. The mixture was filtered through a silica gel plug to remove TBAB, producing a clear yellow filtrate. The solvent was removed under reduced pressure, resulting in a yellowish-white solid. The solid was slurry-washed in heptane (2×500 mL) at ambient temperature, filtered, and air-dried, resulting in very little purification. The heptane-washed solids (294.8 g) were dissolved in anhydrous ethanol (1.0 L) at 73.4° C. The stirred solution was allowed to cool to ambient temperature and was then placed in an ice-water bath for 30 min. The white solids were filtered, slurry-washed in ethanol (2×500 mL), and then vacuum dried first at 40° C. for 15 h and then at 60° C. for 9 h. The resulting solid (178.9 g, 66.0%) was determined to be 43.5% 4-(2-chloro-ethyl)-4′-(3-methoxy-propane-1-sulfonyl)-biphenyl and 50.6% 4-(2-bromo-ethyl)-4′-(3-methoxy-propane-1-sulfonyl)-biphenyl by HPLC peak area. Exact mass calculated for C₁₈H₂₁ClO₃S: 352.09, Found: LCMS m/z=353.1 (M+H⁺); Exact mass calculated for C₁₈H₂₁BrO₃S: 396.04, Found: LCMS m/z (%)=397.2 (M+H^{+ 78}Br, 100), 399.0 (M+H^{+ 80}Br, 97); NMR (400 MHz, DMSO-d₆) δ ppm 1.72-1.83 (m, 2H), 3.07-3.13 (Cl, t, J=7.00 Hz, 2H), 3.12-3.19 (s, 3H), 3.16-3.24 (Br, t, J=7.18, 2H), 3.31-3.39 (m, 4H), 3.25-3.33 (Br, t, J=7.15 Hz, 2H), 3.88-3.95 (Cl, t, J=6.99 Hz, 2H), 7.41-7.48 (d, J=7.09 Hz, 2 H), 7.69-7.74 (d, J=8.13 Hz, 2H), 7.93-7.97 (m, 4H).

### Step D: Preparation of Intermediate 4-(2-Bromo-ethyl)-4′-(3-methoxy-propane-1-sulfonyl)-biphenyl

A total of 161.8 g of the mixture of 4-(2-chloro-ethyl)-4′-(3-methoxy-propane-1-sulfonyl)-biphenyl and 4-(2-bromo-ethyl)-4′-(3-methoxy-propane-1-sulfonyl)-biphenyl prepared in the previous example (43.5% and 50.6% respectively by HPLC peak area) was dissolved in acetonitrile (1.0 L) at ambient temperature. TBAB (88.71 g, 275.2 mmol) and LiBr (95.84 g, 1104 mmol) were added and rinsed into the reaction flask with more acetonitrile (600 mL for a total of 1.6 L). As the resulting mixture was stirred and heated at 60-65° C. under nitrogen for 44 h, additional LiBr was added: 94.75 g (1091 mmol) after 5.5 h; 99.82 g (1149 mmol) after 20 h; and 64.49 g (743 mmol) after 28 h. The reaction mixture was then allowed to cool to 33° C. The liquid phase of the reaction mixture was decanted from the solids, which were rinsed with acetonitrile. The rinse was added to the supernatant, and the solvent was removed under reduced pressure. Deionized water (1.5 L) was added to the evaporation residue. A white solid precipitated, and the resulting mixture was stirred for 1.0 h at ambient temperature. The solids were filtered, washed with deionized water (3×500 mL), and vacuum dried at 40-45° C. for 4 days. The dried solids were dissolved in a mixture of ethyl acetate (3.4 L) and acetonitrile (3.1 L) and, the resulting solution was filtered through a silica gel plug that was subsequently washed with acetonitrile (2×500 mL). The filtrate and washes were combined, and solvent was removed under reduced pressure. The evaporation residue was vacuum dried at 45° C. to provide a white solid (160.2 g, 99.0% recovery) found to be 10.7% 4-(2-chloro-ethyl)-4′-(3-methoxy-propane-1-sulfonyl)-biphenyl and 85.6% 4-(2-bromo-ethyl)-4′-(3-methoxy-propane-1-sulfonyl)-biphenyl by HPLC peak area. Exact mass calculated for C₁₈H₂₁BrO₃S: 396.04, Found: LCMS m/z (%)=397.2 (M+H^{+ 78}Br, 100), 399.0 (M+H^{+ 80}Br, 97); ¹H NMR (400 MHz, DMSO-d₆) δ ppm 1.72-1.83 (m, 2H), 3.12-3.19 (s, 3H), 3.16-3.24 (t, J=7.18 Hz, 2H), 3.31-3.39 (m, 4H) 3.25-3.33 (t, J=7.15 Hz, 2H), 7.41-7.48 (d, J=7.09 Hz, 2H), 7.69-7.74 (d, J=8.13 Hz, 2H), 7.93-7.97 (m, 4H).

### Step E: Preparation of (R)-1-{2-[4′-(3-Methoxy-propane-1-sulfonyl)-biphenyl-4-yl]-ethyl}-2-methyl-pyrrolidine

4-(2-Bromo-ethyl)-4′-(3-methoxy-propane-1-sulfonyl)-biphenyl and 4-(2-chloro-ethyl)-4′-(3-methoxy-propane-1-sulfonyl)-biphenyl (85.55% and 9.31% respectively by HPLC peak area) (198.4 g, 0.499 mol, based on the major starting material) was transferred to a 5 L 3-necked round-bottomed flask fitted with a mechanical stirrer, a temperature probe, a condenser and a nitrogen inlet. (R)-2-Methylpyrrolidine-L-tartrate (95.9 g) was added to the reaction flask followed by acetonitrile (2 L). To this mixture, stirred under nitrogen, potassium carbonate (213.9 g, 1.548 mol) was transferred followed by acetonitrile (380 mL, with washings). The slurry was warmed to 60° C., and water (119 mL) was added slowly. Heating was continued overnight at 60° C. The reaction mixture was cooled to room temperature when the starting 4-(2-bromo-ethyl)-4′-(3-methoxy-propane-1-sulfonyl)-biphenyl was not observed by LC/MS. The reaction mixture was concentrated by distillation of acetonitrile under reduced pressure. The residue was diluted with water (1.2 L) and extracted with ethyl acetate (2×600 mL followed by 500 mL). The combined organic layers were washed with 2 N HCl (2×600 mL followed by 500 mL). The combined aqueous layers were cooled by an ice bath and slowly neutralized with 50% aqueous NaOH (maintaining the internal temperature within 25° C.) and basified further to pH 12-14. The aqueous mixture was extracted with ethyl acetate (2×600 mL followed by 500 mL). The combined ethyl acetate layers were washed with water (2×600 mL followed by 500 mL) until the washings had neutral pH, dried over MgSO₄, filtered and the solvent was removed under reduced pressure. Heptane (300 mL) was added and distilled off to remove residual ethyl acetate. The oily residue was dried overnight under vacuum to afford the crude product (126 g). The product was taken up in heptane (1.6 L), heated to 80° C. and stirred for 1 h. The product was dissolved in hot heptane and the impurities remained as a sticky solid. The solution was filtered hot and heptane was removed under reduced pressure. The residue was dried overnight under vacuum to obtain the product as a pale yellow, waxy solid (113.2 g). HPLC of the product showed 97.63% purity (by peak area). This was dissolved in ethyl acetate (700 mL) and washed with 2 N HCl (500 mL, containing 15% NaCl). Additional 2 N HCl (300 mL), and water (200 mL followed by 100 mL) were required for the separation of the layers. The organic layer was washed with an additional 2 N HCl (400 mL). The combined aqueous layers were washed with ethyl acetate (3×600 mL). HPLC of the acidic aqueous phase showed product purity as 99.09% (by peak area). The aqueous layer was extracted with ethyl acetate (600 mL) and then neutralized by slow addition of 50% aqueous NaOH, while maintaining the temperature below 25° C. with cooling by an ice bath. The aqueous layer was then basified further to pH 12-14. The aqueous mixture was extracted with ethyl acetate (2×600 mL), and the ethyl acetate extracts were washed with water (700 mL) followed by 5% NaCl solution (700 mL). The combined organic phase was dried over MgSO₄, filtered and the solvent was removed under reduced pressure. The residue was suspended in minimum volume of heptane which was distilled off under reduced pressure. The desired product was dried under vacuum to obtain a pale yellow, waxy solid (96.7 g, 48.2%). HPLC purity: 99.04% (by peak area); chiral assay, 99.3% ee. Exact mass calculated for C₂₃H₃₁NO₃S: 401.20, Found: LCMS m/z=401.8 (M+H)⁺, 316.8, 285.2, 207.1, 179.8. NMR (400 MHz, DMSO-d₆) δ ppm 1.02 (d, J=6 Hz, 3H), 1.27 (m, 1H), 1.64 (m, 2H), 1.81 (m, 3H), 2.13 (m, 1H), 2.28 (broad m, 2H), 2.79 (m, 2H), 3.00 (m, 1 H), 3.15 (m, 1H), 3.17 (s, 3H), 3.35 (m, 4H), 7.38 (d, J=8.18 Hz, 2H), 7.68 (d, J=8.24 Hz, 2 H), 7.94 (s, 4H).

### Example 1.2

### Preparation of (R)-1-{2-[4′-(3-Methoxy-propane-1-sulfonyl)-biphenyl-4-yl]-ethyl}-2-methyl-pyrrolidine di-citrate

(R)-1-{2-[4′-(3-Methoxy-propane-1-sulfonyl)-biphenyl-4-yl]-ethyl}-2-methyl-pyrrolidine free base (58.7 g, 0.146 mol) was transferred to a 1 L 3-necked round-bottomed flask, fitted with a mechanical stirrer and a nitrogen inlet. Acetonitrile (600 mL) was added, the mixture was stirred under nitrogen until a clear solution was obtained. To a 250 mL Erlenmeyer flask containing citric acid (59 g, 0.307 mol) was added water (29.5 mL) and the slurry was heated at 60° C. to obtain a clear solution. The warm solution of citric acid was added slowly into the acetonitrile solution of (R)-1-{2-[4′-(3-methoxy-propane-1-sulfonyl)-biphenyl-4-yl]-ethyl}-2-methyl-pyrrolidine free base. Additional water (5 mL) was used to wash the Erlenmeyer flask, and was added to the reaction mixture. After 5 min the solution became cloudy and the mixture was stirred at room temperature for 1.5 h. The mixture was filtered and the solids were washed with acetonitrile (300 mL) and dried in a vacuum oven at 40° C. under house vacuum (−15 Torr) to obtain (R)-1-{2-[4′-(3-methoxy-propane-1-sulfonyl)-biphenyl-4-yl]-ethyl}-2-methyl-pyrrolidine di-citrate (104.6 g, 91%). HPLC purity, 99.15% (by peak area). Chiral assay, 99.1% ee. Exact mass calculated for C₂₃H₃₁NO₃S: 401.20, Found: LCMS m/z=402.0 (M+H)⁺, 316.8, 285.0, 242.5, 207.1, 179.9, 137.0. NMR (400 MHz, DMSO-d₆) δ ppm 1.35 (d, J=6.48 Hz, 3H), 1.61 (m, 1H), 1.79 (m, 2H), 1.95 (m, 2H), 2.18 (m, 1H), 2.61 (m, 8 H), 3.05 (m, 2H), 3.18 (s, 3H), 3.2 (m, 2H), 3.35 (m, 4H), 3.5 (m, 3H), 7.48 (d, J=8.24 Hz, 2H), 7.66 (d, J=8.24 Hz, 2H), 7.96 (s, 4H).

### Example 1.3

### Preparation of Crystalline Form of (R)-1-{2-[4′-(3-Methoxy-propane-1-sulfonyl)-biphenyl-4-yl]-ethyl}-2-methyl-pyrrolidine mono-citrate (FORM 1)

(R)-1-{2-[4′-(3-Methoxy-propane-1-sulfonyl)-biphenyl-4-yl]-ethyl}-2-methyl-pyrrolidine free base (1.01 g, 2.52 mmol) was transferred to a 20 mL scintillation vial. Acetonitrile (10 mL) was added, the mixture was stirred until a clear solution was obtained. To another vial containing citric acid (510 mg, 2.66 mmol) was added water (0.5 mL) and the slurry was heated to obtain a clear solution. The citric acid solution was added into the acetonitrile solution of (R)-1-{2-[4′-(3-methoxy-propane-1-sulfonyl)-biphenyl-4-yl]-ethyl}-2-methyl-pyrrolidine free base. No precipitation was initially observed, 1-10 mg of (R)-1-{2-[4′-(3-methoxy-propane-1-sulfonyl)-biphenyl-4-yl]-ethyl}-2-methyl-pyrrolidine di-citrate was added resulting in a cloudy/precipated solution. This solution was placed into an ice bath to further facilitate precipitation. The mixture was filtered and the solid cake was washed with acetonitrile and placed on lyophilzer to dry overnight to obtain (R)-1-{2-[4′-(3-methoxy-propane-1-sulfonyl)-biphenyl-4-yl]-ethyl}-2-methyl-pyrrolidine mono-citrate. Chiral assay, 98.9% ee. Exact mass calculated for C₂₃H₃₁NO₃S: 401.20, Found: LCMS m/z=402.2 (M+H)⁺. ¹H NMR (400 MHz, DMSO-d₆) δ ppm 1.30 (d, J=6.06 Hz, 3H), 1.57 (dd, J=12.63, 8.08 Hz, 1H), 1.73-1.84 (m, 2H), 1.92 (m, 2H), 2.15 (m, 1H), 2.55 (m, 4H), 2.99 (m, 2H), 3.05 (br s, 1H), 3.17 (s, 3H), 3.30 (br s, 1H), 3.35 (t, J=6.32 Hz, 4H), 3.44 (m, 2H), 3.52 (m, 2H), 7.47 (d, J=8.59 Hz, 2H), 7.76 (d, J=8.08 Hz, 2H), 7.96 (s, 4H).

### Example 1.4a

### Preparation of Crystalline Form of (R)-1-{2-[4′-(3-Methoxy-propane-1-sulfonyl)-biphenyl-4-yl]-ethyl}-2-methyl-pyrrolidine mono-citrate (FORM 2)

(R)-1-{2-[4′-(3-Methoxy-propane-1-sulfonyl)-biphenyl-4-yl]-ethyl}-2-methyl-pyrrolidine free base (1.6 g) was dissolved in acetone (20 mL). Addition of citric acid (about 0.015 mL of a 4.15 M aqueous solution) to an aliquot of the acetone solution of (R)-1-{2-[4′-(3-methoxy-propane-1-sulfonyl)-biphenyl-4-yl]-ethyl}-2-methyl-pyrrolidine free base (0.31 mL) gave a solution which was evaporated to dryness. To the resulting thick oil was added IPA (about 0.3 mL) before heating briefly to about 50° C. in a ReactiTherm to get the oil into solution. The solution was allowed to cool down and stand at room temperature overnight. The precipitate was filtered and slurried in IPA for 2 days before it was recovered by centrifuge filtration and air dried. The solid was characterized by PXRD and DSC.

### Example 1.4b

### Preparation of Crystalline Form of (R)-1-{2-[4′-(3-Methoxy-propane-1-sulfonyl)-biphenyl-4-yl]-ethyl}-2-methyl-pyrrolidine mono-citrate (FORM 2)

(R)-1-{2-[4′-(3-Methoxy-propane-1-sulfonyl)-biphenyl-4-yl]-ethyl}-2-methyl-pyrrolidine free base is dissolved in isopropyl alcohol. Citric acid is added to the solution of the free base. The resulting mixture is stirred. The resulting precipitate is filtered.

### Example 1.5

### Preparation of (R)-1-{2-[4′-(3-Methoxy-propane-1-sulfonyl)-biphenyl-4-yl]-ethyl}-2-methyl-pyrrolidine di-citrate (Method 1)

(R)-1-{2-[4′-(3-Methoxy-propane-1-sulfonyl)-biphenyl-4-yl]-ethyl}-2-methyl-pyrrolidine free base (6.14 g) was dissolved in acetonitrile (61.4 mL, 10 vol) at room temperature. A solution of citric acid (6.17 g) in water (3.08 mL) was prepared by warming up the mixture at about 50° C. The warm aqueous solution of citric acid was subsequently added to the acetonitrile solution of (R)-1-{2-[4′-(3-methoxy-propane-1-sulfonyl)-biphenyl-4-yl]-ethyl}-2-methyl-pyrrolidine free base while stirring. The solution was stirred for 15 minutes at room temperature before it became cloudy and the salt started precipitating. The mixture thickened over time and was stirred for 1.5 hours before it was filtered. The residue was washed with ACN and dried overnight in a vacuum oven at 40° C. under house vacuum to yield (R)-1-{2-[4′-(3-methoxy-propane-1-sulfonyl)-biphenyl-4-yl]-ethyl}-2-methyl-pyrrolidine di-citrate (10.62 g, 88%). HPLC purity, 98.85% (by peak area). NMR (400 MHz, DMSO-d₆) δ ppm 1.30 (d, J=6.06 Hz, 3H), 1.57 (dd, J=12.63, 8.08 Hz, 1H), 1.73-1.84 (m, 2H), 1.92 (m, 2H), 2.15 (m, 1H), 2.55 (m, 4H), 2.99 (m, 2H), 3.05 (br s, 1H), 3.17 (s, 3H), 3.30 (br s, 1H), 3.35 (t, J=6.32 Hz, 4 H), 3.44 (m, 2H), 3.52 (m, 2H), 7.47 (d, J=8.59 Hz, 2H), 7.76 (d, J=8.08 Hz, 2H), 7.96 (s, 4 H).

### Example 1.6

### Preparation of 2-(4′-(3-methoxypropylsulfonyl)biphenyl-4-yl)ethyl methanesulfonate

### Step A: Preparation of 2-(4′-(Chlorosulfonyl)biphenyl-4-yl)acetic Acid By Chlorosulfonylation of 2-(Biphenyl-4-yl)acetic acid

A mixture of 4-biphenylacetic acid (1.50 kg, 7.07 mol) and trifluoroacetic acid (10.5 L, 16.1 kg, 7 volumes) was stirred at 21° C. With external cooling, chlorosulfonic acid (3.28 L, 5.76 kg, 49.5 mol, 7 eq.) was added over 2 h maintaining the internal temperature between 21-25° C. After the addition was completed, the reaction mixture was stirred at 20-21° C. for 20 h. The reaction mixture was divided into two equal portions (2×11.2 kg) and quenched batch-wise as described below.

A solution of water (4 L) and acetic acid (1.30 kg) was cooled to 6° C. With external cooling, the reaction mixture (11.2 kg) was slowly added to the stirred quench solution over 2.5 h maintaining the temperature below 22° C. The mixture was stirred for an additional 30 min and the solid was collected by filtration. The filter-cake was washed with water (3×1.5 L) and dried under suction providing sulfonyl chloride as a wet-cake. This procedure was repeated for the second portion and afforded a combined 6.34 kg of 2-(4′-(chlorosulfonyl)biphenyl-4-yl)acetic acid as a wet-cake. HPLC purity, 94% (by peak area). Mass calculated for C₁₄H₁₁ClO₄S: 310.0, Found: LCMS m/z (%)=311.1 ([M+H]⁺, 40), 265.0 (100); NMR (400 MHz, CDCl₃): δ 8.10 (d, J=8.8 Hz, 2H), 7.80 (d, J=8.8 Hz, 2H), 7.61 (d, J=8.4 Hz, 2H), 7.45 (d, J=8.4 Hz, 2H), 3.75 (s, 2H).

### Step B: Preparation of 2-(4′-(3-Methoxypropylsulfonyl)biphenyl-4-yl)acetic Acid By Alkylation of 2-(4′-(Chlorosulfonyl)biphenyl-4-yl)acetic Acid

A solution of water (12.0 L), sodium sulfite (1.22 kg, 3.0 eq.), and sodium phosphate, dibasic (1.14 kg, 2.5 eq.) was degassed with nitrogen for at least 30 min. The wet-cake containing 2-(4′-(chlorosulfonyl)biphenyl-4-yl)acetic acid (1.00 kg, 3.21 mol) was charged in one portion. After sparging again with nitrogen for at least 10 min, the contents were heated at 60° C. for 1 h. When the reaction was determined complete, tetrabutylammonium bromide (0.10 kg, 0.10 eq.) and KI (0.05 kg, 0.10 eq.) were charged to the reaction solution. The mixture was heated at 70-75° C. and 1-bromo-3-methoxypropane (2.02 kg, 4.10 equiv) over 12 h. The mixture was cooled to ambient temperature and 50 wt % aqueous NaOH solution (1.33 kg) was added; the pH of the reaction solution was adjusted to 13-14. The mixture was heated at 80° C. for at least 1 h. The mixture was cooled to 60° C. and a solution of aqueous H₂SO₄ (50 v/v %, 1.20 kg) was charged adjusting the pH to 4.5-5. The contents were then partitioned with 2-methyltetrahydrofuran (2-MeTHF; 4.3 kg) at 60-65° C. and the biphasic mixture was cooled to 25° C. The phases were separated and the organic phase was washed with water (2.00 kg). The organic phase was concentrated at 40-50° C. under reduced pressure to remove the majority of solvent. The concentrate was diluted with i-PrOH (1.2 kg) and re-concentrated to remove most of the solvent. The concentrate was diluted with i-PrOH (2.36 kg) and heated at 70-80° C. to dissolve the solid. The solution was cooled to 20° C. and aged at 20° C. for at least 2 h. The solid was collected by filtration and the filter-cake was washed with cold i-PrOH (1.37 kg). The filter-cake was dried by suction and then further dried under reduced pressure (30° C./20 torr) to afford 2-(4′-(3-methoxypropylsulfonyl)biphenyl-4-yl)acetic acid (0.896 kg, 80% yield) as an off-white powder. HPLC purity, 98.7% (by peak area). KF: 0.4 wt % H₂O. Mass calculated for: C₁₈H₂₀O₅S: 348.1, Found: LCMS m/z (%)=349.4 ([M+H]⁺, 32), 317.1 ([M+H—CH₃OH]⁺, 100); ¹H NMR (400 MHz, CDCl₃) δ 7.97 (d, J=8.6 Hz, 2H), 7.75 (d, J=8.6 Hz, 2H), 7.60 (d, J=8.3 Hz, 2H), 7.42 (d, J=8.3 Hz, 2H), 3.74 (s, 2H), 3.46 (t, J=6.0 Hz, 2H), 3.29 (s, 3H), 3.22-3.26 (m, 2H), 2.00-2.07 (m, 2H).

### Step C. Preparation of 2-(4′-(3-Methoxypropylsulfonyl)biphenyl-4-yl)ethanol By Reduction of 2-(4′-(3-Methoxypropylsulfonyl)biphenyl-4-yl)acetic Acid

A mixture of 2-(4′-(3-methoxypropylsulfonyl)biphenyl-4-yl)acetic acid (1.00 kg, 2.87 mol) and NaBH₄ (163 g, 1.50 eq.) was diluted with THF (5.42 kg). The mixture was cooled at 5-10° C. and BF₃.OEt₂ (0.62 kg, 1.50 eq.) was added while maintaining the temperature below 15° C. After the addition was completed, the reaction mixture was agitated at 0-5° C. for an additional 1.5 h. After the reaction was completed, acetone (1.74 kg) was charged and the reaction mixture was heated at 60-65° C. for 2 h. Aqueous NaOH solution (50 wt %, 1.74 kg) was slowly added to the reaction mixture and the contents were heated at 80° C. for 2 h. The mixture was cooled to 20-25° C. and concentrated under reduced pressure to 20% of the original volume. The concentrate was partitioned between water (4.00 kg) and i-PrOAc (8.72 kg), heated at 50° C. for 1 h, and the phases were separated. The organic phase was washed with water (2×3.00 L). The organic phase was concentrated under reduced pressure to about ⅓ volume (3.6 L). The concentrate was heated at 60° C., diluted with heptane (4.00 kg), cooled to 0-5° C., and stirred at 0-5° C. for 2 h. The solid was collected by filtration, dried by suction, and dried further under reduced pressure (45° C./20 torr) to afford 2-(4′-(3-methoxypropylsulfonyl)biphenyl-4-yl)ethanol (0.905 kg, 94%) as an off-white powder. The purity was 99.0 area % by HPLC. KF: 0.19 wt % water. Mass calculated for: C₁₈H₂₂O₄S: 334.1. Found: LCMS m/z (%)=335.5 ([M+H]⁺, 58), 303.4 ([M+H—CH₃OH]⁺, 100); ¹H NMR (400 MHz, CDCl₃): δ 7.97 (d, J=8.5 Hz, 2H), 7.76 (d, J=8.5 Hz, 2H), 7.57 (d, J=8.2 Hz, 2H), 7.37 (d, J=8.2 Hz, 2H), 3.94 (t, J=6.5 Hz, 2H), 3.45 (t, J=6.0 Hz, 2H), 3.29 (s, 3H), 3.22-3.26 (m, 2H), 2.95 (t, J=6.5 Hz, 2H), 2.00-2.07 (m, 2H), 1.49 (bs, 1H).

### Step D. Preparation of 2-(4′-(3-methoxypropylsulfonyl)biphenyl-4-yl)ethyl methanesulfonate By Methylsulfonylation of 2-(4′-(3-methoxypropylsulfonyl)biphenyl-4-yl)ethanol

A solution of 2-(4′-(3-methoxypropylsulfonyl)biphenyl-4-yl)ethanol (12.1 kg, 36.2 mol), acetonitrile (ACN, 15.0 kg), methyl t-butyl ether (MTBE, 57 kg), and N,N-diisopropylethylamine (6.68 kg, 1.40 eq.) was cooled at 0 to 5° C. To the cold solution, MsCl (5.74 kg, 1.40 eq.) was added over 50 min at a rate to maintain the temperature at 0-5° C. After the addition was completed, the solution was stirred at 0-5° C. for an additional 2 h. The solution was quenched with water (30 kg, 2.5 volumes) while maintaining the temperature from 0-10° C. The temperature of the quenched mixture was raised to 25° C., and the phases were separated. The organic phase was washed with water (30 kg) at 25-30° C. and washed again with water (30 kg) at 35° C., separating the phases after each washing. The organic phase was diluted with methyl t-butyl ether (36 kg) and heated at 55-60° C. for 1 h. The mixture was cooled to 0-5° C. over 2 h and held at 0-5° C. for 1 h. The solid was collected by filtration, the filter-cake was washed with methyl t-butyl ether (19 kg), dried with suction, and further dried at under reduced pressure (45° C./15 torr) to afford the title compound (12.4 kg, 82.9%) as a white powder. Mass calculated for: C₁₉H₂₄O₆S₂: 412.1, Found: LCMS m/z (%)=413.5 ([M+H]⁺, 39), 381.2 (M+H—CH₃OH]⁺, 100); ¹H NMR (400 MHz, CDCl₃): δ 7.97 (d, J=8.4 Hz, 2H), 7.76 (d, J=8.4 Hz, 2 H), 7.59 (d, J=8.2 Hz, 2H), 7.37 (d, J=8.1 Hz, 2H), 4.48 (t, J=6.8 Hz, 2H), 3.45 (t, J=5.9 Hz, 2H), 3.29 (s, 3H), 3.26-3.22 (m, 2H), 3.14 (t, J=6.8 Hz, 2H), 2.94 (s, 3H), 2.06-1.99 (m, 2H).

### Example 1.7

### Preparation of (R)-1-{2-[4′-(3-Methoxy-propane-1-sulfonyl)-biphenyl-4-yl]-ethyl}-2-methyl-pyrrolidine And Conversion to the Di-citrate

### Method 1

### Step A: Preparation of (R)-1-{2-[4′-(3-Methoxy-propane-1-sulfonyl)-biphenyl-4-yl]-ethyl}-2-methyl-pyrrolidine

A biphasic mixture of 2-(4′-(3-methoxypropylsulfonyl)biphenyl-4-yl)ethyl methanesulfonate (1.019 kg, 2.47 mmol), anhydrous K₂CO₃ (1.024 kg, 3 eq.), (R)-2-methylpyrrolidine L-tartrate (814 g, 1.4 eq.), acetonitrile (8.15 L, 8 volumes), and water (2.86 L,

2.8 volumes) was heated at 70° C. for 24 h. After the reaction was completed, the mixture was

concentrated by distillation, under reduced pressure, to remove most of the acetonitrile (7.7 L). The concentrate was partitioned with 2-butanone (methyl ethyl ketone, MEK, 3.05 L, 3 volumes), the resultant phases were separated, and the organic phase was washed with a solution of 20 wt % NaCl in water (3.0 kg). The organic phase was distilled to azeotropically remove water. After 2.5 L of distillate was removed, the concentrate was diluted with 2-butanone (2.5 L).

### Step B: Preparation of (R)-1-{2-[4′-(3-Methoxy-propane-1-sulfonyl)-biphenyl-4-yl]-ethyl}-2-methyl-pyrrolidine Di-citrate

Anhydrous citric acid (1.043 kg, 2.2 eq.) and methanol (3.06 L, 3 volumes) were charged to the organic phase. The mixture was warmed at 60° C. and diluted with 2-butanone (10 volumes) while maintaining the temperature between 55-60° C. The mixture was cooled to 0-5° C. over 5 h and held at 0-5° C. for 4 h. The solid was collected by filtration and the filter-cake was washed with 2-butanone (2×1.5 L). The filter-cake was dried with suction and further dried under reduced pressure (45° C./10 torr) to afford the title compound as a white powder (1.642 kg, 85%). Analytical data from a representative batch: HPLC purity was 99.7 area %. Exact mass calculated for: C₂₃H₃₂NO₃S⁺402.2097, found: LCMS m/z=402.2021 [M+H]⁺; ¹H NMR (400 MHz, DMSO-d₆): δ 10.91 (bs, 6H), 7.95 (s, 4H), 7.76 (d, J=8.2 Hz, 2H), 7.48 (d, J=8.2 Hz, 2H), 3.62-3.56 (m, 1H), 3.54-3.41 (m, 3H), 3.36-3.32 (m, 4H), 3.24-3.15, m, 2H), 3.17 (s, 3H), 3.10-2.96 (m, 2H), 2.61 (dd, J=35.0, 15.2 Hz, 8H), 2.23-2.14 (m, 1H), 1.99-1.90 (m, 2H), 1.82-1.75 (m, 2H), 1.66-1.56 (m, 1H), 1.35 (d, f=6.6 Hz, 3H).

### Method 2

### Step A: Preparation of (R)-1-{2-[4′-(3-Methoxy-propane-1-sulfonyl)-biphenyl-4-yl]-ethyl}-2-methyl-pyrrolidine

A biphasic mixture of 2-(4′-(3-methoxypropylsulfonyl)biphenyl-4-yl)ethyl methanesulfonate (12.2 kg, 29.6 mol), anhydrous K₂CO₃ (12.3 kg, 3 eq.), (R)-2-methylpyrrolidine L-tartrate (9.76 kg, 1.4 eq.), acetonitrile (97.5 L, 8 volumes), and water (34.2 L, 2.8 volumes) was heated at 70-75° C. for 20 h. After the reaction was completed, the mixture was concentrated by distillation, under reduced pressure, to remove most of the acetonitrile. The concentrate was partitioned between methyl ethyl ketone (38.7 L, 3 volumes) and additional water (7.7 L, 0.6 volumes). The resultant phases were separated and the organic phase was washed with a solution of 20 wt % NaCl in water (36.8 kg). The organic phase was clarified by recirculation through in-line filters and diluted with 2-butanone (7.8 L, 0.6 volumes).

### Step B: Preparation of (R)-1-{2-[4′-(3-Methoxy-propane-1-sulfonyl)-biphenyl-4-yl]-ethyl}-2-methyl-pyrrolidine Di-citrate

A previously prepared solution of anhydrous citric acid (12.4 kg, 2.2 eq.) and methanol (36.7 L, 3 volumes) was charged to the organic phase. The mixture was warmed at 60-65° C., cooled at 50-55° C., and diluted with 2-butanone (121 L, 10 volumes) while maintaining the temperature between 55-60° C. The reactor contents were warmed to 62° C. and then cooled to 37° C. over 1 h. The temperature was rapidly cooled to 10° C. to induce crystallization. The resultant mixture was further cooled to 0-5° C. and aged for 9 h. An attempt to collect the solid by filtration failed. The portion of wet cake that was collected was redissolved in hot MeOH (90 L, 7 volumes) and added back to the unfiltered mixture. The mixture was distilled under reduced pressure and recharged with 2-butanone until the desired 20 wt % methanol in 2-butanone (16.5 volumes) was achieved. After the solvent ratio and volume were adjusted back to their desired values the reactor contents were cooled to 30° C., seeded, and aged at 30° C. The contents were further cooled to and aged at 0-5° C. The solid was collected by filtration, the filter-cake was washed with 2-butanone (4×2 volumes), and dried under reduced pressure with heat and a nitrogen sweep to afford a 1st crop of the title compound (12.6 kg, 54.0%) as a white powder containing a low level of mono-methyl citrate. The mother liquor and washings were combined and concentrated under reduced pressure to 12 wt % methanol in 2-butanone (˜6 volumes). After cooling to and aging at 0-5° C., the solid was collected by filtration, washed with 2-butanone (3×1 volume), and dried under reduced pressure at 50° C. to afford a second crop (4.12 kg, 17.7%) of the title compound as a white powder containing a low level of mono-methyl citrate.

### Step C: Purification of (R)-1-{2-[4′-(3-Methoxy-propane-1-sulfonyl)-biphenyl-4-yl]-ethyl}-2-methyl-pyrrolidine Di-citrate

A portion of the crude (R)-1-{2-[4′-(3-Methoxy-propane-1-sulfonyl)-biphenyl-4-yl]-ethyl}-2-methyl-pyrrolidine di-citrate (200 g, 0.485 mol) was slurried with anhydrous citric acid (4.89 g, 0.10 eq.) in water (60 mL, 0.3 volumes) and acetonitrile (1.94 L, 9.7 volumes) and heated at 60-65° C. for 48 h. The slurry was cooled to 0-5° C. over 2.5 h, aged at 0-5° C. for 2 h, and the solid was collected by filtration. The filter-cake was washed with acetonitrile (800 mL, 4 volumes), allowed to dry by suction, and dried further under reduced pressure at 45-50° C. to afford the title compound as a white, crystalline solid (188.4 g, 94.2%). HPLC analysis of the counter ions showed 99.5 area % citric acid and 0.39 area % mono-methyl citrate. HPLC analysis of the parent showed a purity of 99.8 area %.

### Method 3

### Step A: Preparation of (R)-1-{2-[4′-(3-Methoxy-propane-1-sulfonyl)-biphenyl-4-yl]-ethyl}-2-methyl-pyrrolidine

A biphasic mixture of 2-(4′-(3-methoxypropylsulfonyl)biphenyl-4-yl)ethyl methanesulfonate, anhydrous K₂CO₃ (3 eq.), (R)-2-methylpyrrolidine L-tartrate (1.4 eq.), acetonitrile (8 volumes), and water (2.8 volumes) is heated at 70° C. for 24 h. After the reaction is completed, the mixture is concentrated by distillation, under reduced pressure, to remove most of the acetonitrile. The concentrate is diluted with a water-immiscible organic solvent (e.g. ethyl acetate or methyl t-butyl ether; 3 volumes), the resultant phases are separated, and the organic phase is washed with water (3 volumes). The organic phase is concentrated by distillation to remove most of the solvent and acetonitrile (9.7 volumes) is added.

### Step B: Preparation of (R)-1-{2-[4′-(3-Methoxy-propane-1-sulfonyl)-biphenyl-4-yl]-ethyl}-2-methyl-pyrrolidine Di-citrate

Anhydrous citric acid (2.2 eq.) and water (0.3 volumes) are charged to the organic phase. The resultant mixture is warmed at 60° C. and heated at 60-65° C. for 12-48 h. The slurry is cooled to 0-5° C. over 2-4 h, aged at 0-5° C. for 2 h, and the solid is collected by filtration. The filter-cake is washed with acetonitrile (3×4 volumes), allowed to dry by suction, and dried further under reduced pressure at 40-50° C. to afford the title compound.

### Method 4

### Step A: Preparation of (R)-1-{2-[4′-(3-Methoxy-propane-1-sulfonyl)-biphenyl-4-yl]-ethyl}-2-methyl-pyrrolidine

A biphasic mixture of 2-(4′-(3-methoxypropylsulfonyl)biphenyl-4-yl)ethyl methanesulfonate (20.0 g, 48.5 mmol), anhydrous K₂CO₃ (20.1 g, 3 eq.), (R)-2-methylpyrrolidine L-tartrate (16.0 g, 1.4 eq.), acetonitrile (160 mL), and water (56 mL) was heated at 65° C. for 16 h. HPLC showed 6% mesylate remaining with 11.2% of the corresponding styrene. Water (40 mL) was added and the acetonitrile was removed by distillation at 95° C. (160 mL collected). The residue was stirred at room temperature for two days. Heating was then resumed and a further fraction of solvent (15 mL) was removed by distillation (head temperature 86° C.). The residue was cooled to 30° C. and diluted with methyl t-butyl ether (160 mL). The resultant phases were separated and the organic phase was washed with water (2×40 mL). The organic phase was filtered through Celite® to afford a light yellow solution, which was heated to 55° C. to remove most of the methyl t-butyl ether by distillation, azeotroping to remove water. Acetonitrile (369.6 mL) was added and the mixture was heated to remove the remaining methyl t-butyl ether by distillation to a maximum head temperature of 80° C. The mixture was then cooled to 70° C.

### Step B: Preparation of (R)-1-{2-[4′-(3-Methoxy-propane-1-sulfonyl)-biphenyl-4-yl]-ethyl}-2-methyl-pyrrolidine Di-citrate

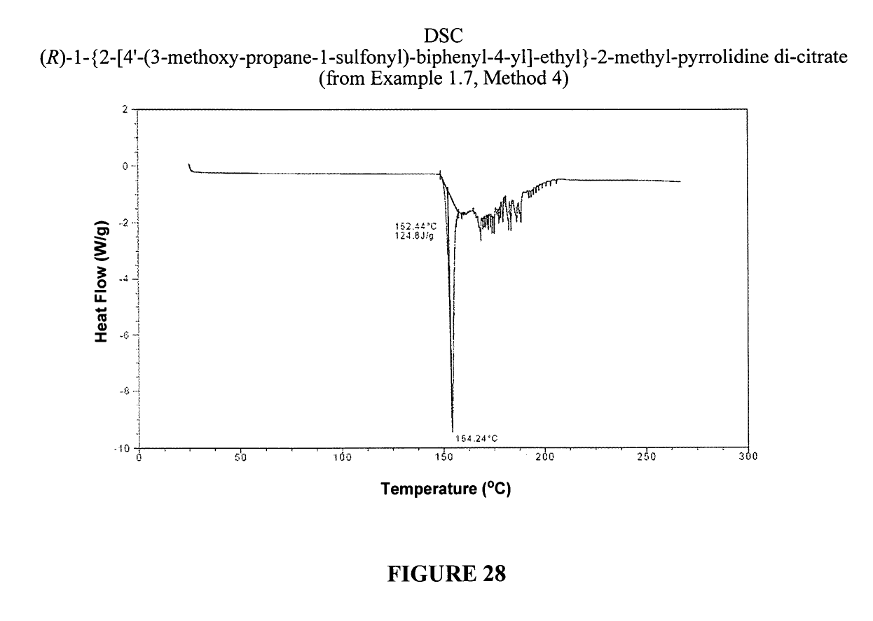

A thin syrupy solution of citric acid was prepared by dissolving citric acid (20.5 g) in water (11.4 mL) with warming. The citric acid solution was added to the solution from Step A at 70° C. and the mixture was allowed to cool to 60° C. over 30 min. The mixture was held at 60° C. for 2 h during which time it became a thick paste. Water (5 mL) was added and the mixture was heated to 70° C. to form a clear solution. The mixture was then heated to reflux temperature (77° C.) and a portion of the solvent (38 mL) was removed by distillation. The solution was cooled to 75° C. and seed crystals (0.38 g) were added but dissolved. The solution was cooled to 65° C. and a second portion of seed crystals (0.38 g) was added. The mixture was then cooled to 60° C. and held at that temperature for 16 h; cooled to 50° C. over 20 min and held at that temperature for 1 h; cooled to 40° C. and held for 1 h; cooled to 30° C. and held for 30 min; and finally cooled to 0-5° C. over 30 min and held at that temperature for 30 min. The solid was collected by filtration, washed with 3% water/acetonitrile (2×40 mL), dried by suction for 10 min and stirred to a uniform powdery consistency. Drying was continued by suction overnight to leave a fine white powder (31.07 g, 81%). HPLC purity was 99.7 area %. For DSC see FIG. 28.

### Example 1.8a

### Preparation of (R)-1-{2-[4′-(3-Methoxy-propane-1-sulfonyl)-biphenyl-4-yl]-ethyl}-2-methyl-pyrrolidine maleate

(R)-1-{2-[4′-(3-Methoxy-propane-1-sulfonyl)-biphenyl-4-yl]-ethyl}-2-methyl-pyrrolidine free base (1.6 g) was dissolved in acetone (20 mL). Addition of maleic acid (about 0.015 mL of a 4.15 M aqueous solution) to an aliquot of the acetone solution of (R)-1-{2-[4′-(3-methoxy-propane-1-sulfonyl)-biphenyl-4-yl]-ethyl}-2-methyl-pyrrolidine free base (0.31 mL) gave a solution which was evaporated to dryness. To the resulting thick oil was added IPA (about 0.3 mL) before heating briefly to about 50° C. in a ReactiTherm to get the oil into solution. The solution was allowed to cool down and stir at room temperature overnight. The precipitate was collected by centrifuge filtration and air dried. NMR (400 MHz, DMSO-d₆) δ ppm 1.40 (d, J=6.27 Hz, 3H), 1.58-1.68 (m, 1H), 1.79-1.86 (m, 2H), 1.90-2.07 (m, 1H), 2.99-3.15 (m, 2H), 3.20 (s, 3H), 3.23-3.42 (m, 7H), 3.45-3.70 (m, 3H), 6.05 (s, 4H), 7.51 (d, J=8.16 Hz, 2H), 7.79 (d, J=8.28 Hz, 2H), 7.99 (s, 4H).

### Example 1.8b

### Preparation of (R)-1-{2-[4′-(3-Methoxy-propane-1-sulfonyl)-biphenyl-4-yl]-ethyl}-2-methyl-pyrrolidine maleate

(R)-1-{2-[4′-(3-Methoxy-propane-1-sulfonyl)-biphenyl-4-yl]-ethyl}-2-methyl-pyrrolidine free base (1.6 g) was dissolved in acetone (20 mL). Addition of maleic acid (about 0.015 mL of a 4.15 M aqueous solution) to an aliquot of the acetone solution of (R)-1-{2-[4′-(3-methoxy-propane-1-sulfonyl)-biphenyl-4-yl]-ethyl}-2-methyl-pyrrolidine free base (0.31 mL) gave a solution which was evaporated to dryness. To the resulting thick oil was added IPA (about 0.3 mL) before heating briefly to about 50° C. in a ReactiTherm to get the oil into solution. The solution was allowed to cool down and stir at room temperature overnight. Precipitation occurred during cooling, or optionally a maleate seed crystal can be added to assist in precipitation. The precipitate was collected by centrifuge filtration and air dried to provide (R)-1-{2-[4′-(3-methoxy-propane-1-sulfonyl)-biphenyl-4-yl]-ethyl}-2-methyl-pyrrolidine maleate.

### Example 1.9

### Preparation of (R)-1-{2-[4′-(3-Methoxy-propane-1-sulfonyl)-biphenyl-4-yl]-ethyl}-2-methyl-pyrrolidine hydrochloride

(R)-1-{2-[4′-(3-Methoxy-propane-1-sulfonyl)-biphenyl-4-yl]-ethyl}-2-methyl-pyrrolidine free base was obtained by neutralization of the (R)-1-{2-[4′-(3-methoxy-propane-1-sulfonyl)-biphenyl-4-yl]-ethyl}-2-methyl-pyrrolidine di-citrate (2.0 g) with 0.5 N aqueous solution of NaOH (25 mL). After extraction with isopropyl acetate, the organics were separated, washed with water, dried over MgSO₄, filtered and concentrated to afford a colorless viscous oil. The oil (0.2 g to 0.5 g) was dissolved in diethylether (20 mL to 50 mL) before an ethereal solution of 1M HCl was added (to pH 1) to afford a sticky waxy semi-solid. After overnight stirring of the semi-solid in a closed system, a free flowing white solid was obtained, filtered under a N₂ blanket and rinsed with diethyl ether.

### Example 2a

### Powder X-Ray Diffraction Analysis (Method 1)

Data were collected using a PANalytical X'Pert Plus Powder X-ray diffractometer in the 2θ geometry. Samples were analyzed using a spinning sample stage, illuminated with Cu Kα radiation (λ=˜1.54 Å) maintained at 45 kV and 40 mA and measured from 5.0 to 40.0° 20. The beam aperture was controlled using tube divergence and anti-scatter slits of ⅛° and ¼° respectively, while the detector anti-scatter slit was set at 0.5 mm. Data were collected from 5° to 40° two-theta (2θ) in continuous scan mode with a step size of 0.02. The samples were prepared using PANalytical round top-loading stainless steel sample holders, and were fitted with low background inserts.

### Example 2b

### Powder X-Ray Diffraction Analysis (Method 2)

Powder X-ray Diffraction (PXRD) data were collected on an X'Pert PRO MPD powder diffractometer (PANalytical, Inc.) with a Cu source set at 45 kV and 40 mA, a Ni-filter to remove Cu Kβ radiation, and an X′Celerator detector. The samples were analyzed using a spinning-sample stage. Scans cover the range of 5 to 40° 2θ. A continuous scan mode is used with a step size of 0.0170° 2θ. Diffraction data are viewed and analyzed with the X'Pert Data Viewer Software, version 1.0a and X'Pert HighScore Software, version 1.0b

### Example 3a

### Thermal Analysis (Method 1)

Differential Scanning Calorimetry (DSC)

Data were collected using TA Instruments Q1000 Differential Scanning Calorimetry (DSC). Analysis was conducted by sealing samples in an aluminum pan with a crimped lid and heating, under nitrogen, from 25 to 170° C. at 10° C./min.

Thermogravimetric Analysis (TGA)

Data were collected using TA Instruments Q500 TGA. Analysis was conducted by heating samples in an open aluminum pan under nitrogen from 30 to 250° C. at a rate of 10° C./min.

### Example 3b

### Thermal Analysis (Method 2)

Differential Scanning Calorimetry (DSC)

Differential Scanning Calorimetry (DSC) was performed on a TA instruments, Inc. DSC Q2000. The samples are heated at 10° C./min from about 25° C. to about 210° C. Thermal events are calculated using the Universal Analysis 2000 software, version 4.1D, Build 4.1.0.16.

Thermogravimetric Analysis (TGA)

Thermal Gravimetric Analysis (TGA) was performed on the TA Instruments, Inc. TGA Q500. Sample scans are performed at 10° C./min from about 25° C. to about 200° C.-250° C. Sample is placed into an open sample pan, previously tared on the TGA balance. Thermal events are calculated using the Universal Analysis 2000 software, version 4.1D, Build 4.1.0.16.

### Example 4a

### Vapor Sorption Analysis (Method 1)

Dynamic vapor sorption analysis was conducted using a VTI-SGA100. Samples were placed in an open pan inside the instrument and allowed to dry at a relative humidity (RH) of 0%. The sample was considered sufficiently dry when a change in mass over time (dm/dt) was less than 0.04%. The subsequent experiment which, started immediately after drying, was as follows: RH increased in 5% RH steps from 0-95% followed by decreasing RH steps from 95-5%. A change in dm/dt of 0.002% was used to determine when equilibration at any given step was achieved prior to the instrument moving to the next relative humidity step.

### Example 4b

### Vapor Sorption Analysis (Method 2)

Hygroscopicity was measured using a dynamic moisture-sorption analyzer, VTI Corporation, SGA-100. The sample was placed as-is in a tared sample holder on the VTI balance. A drying step was run at 40° C. and 1% RH for 20 minutes. The isotherm conditions are 25° C. with steps of 20% RH from 10% RH up to 90% RH and back to 10% RH. Weight is checked every 5 minutes. Consecutive % weight change of <0.01% or 2 hours, whichever occurs first, is required before continuing to the next step.

### Example 5

### FTIR

Experimental conditions: All samples (powdered) were prepared by placing approximately 5 milligrams directly on the ZnSe crystal at the center of the single-bounce

MIRacle ATR accessory. The caliper accessory was used to apply gentle pressure to the material.

Spectra were obtained for the spectral region from 4000 cm⁻¹ down to 600 cm⁻¹ with a resolution of 4 cm⁻¹. A 60 second delay prior to data collection was used prior to the collection of 32 scans. Each interferogram was collected with 16,384 points, apodized using a Blackman-Harris 3-term polynomial correction, zero-filled to 32 K points, then the power spectrum was taken. In order to correct the slope in the baseline, the software scattering baseline correction was used.

### Example 6

### RAMAN Spectroscopy

Raman spectra of the samples were recorded using the Thermo Nicolet NXR6700 FT-Raman System using the microstage accessory. The instrument consists of a NdYAg laser operating at a wavelength of 1064 nm, an interferometer with a calcium fluoride beam-splitter, and an InGaAs detector. No background spectrum was required, and the Raman spectra were recorded by placing approximately 1 mg of each sample directly into the powder cup on the sample stage.

In order to collect the spectra, 1024 transients of an interferogram containing 8192 points was acquired with 4 cm⁻¹ resolution. The spectrum was recorded from 100 cm⁻¹ to 3700 cm⁻¹. The interferogram was apodized with a Happ-Genzel function and the data was zero-filled once prior to the application of a power spectrum for phase correction.

### Example 7

### HPLC Determination of the Stoichiometry of (R)-1-{2-[4′-(3-Methoxy-propane-1-sulfonyl)-biphenyl-4-yl]-ethyl}-2-methyl-pyrrolidine Mono-citrate and Di-citrate

Two HPLC methods were used to characterize the stoichiometry in this study. Method 1 was used to quantify the content of (R)-1-{2-[4′-(3-methoxy-propane-1-sulfonyl)-biphenyl-4-yl]-ethyl}-2-methyl-pyrrolidine, i.e., Cmpd (I), in each sample. Method 2 was used to quantify the citric acid content.

### Method 1

Column: Ace 3 C18 (Advanced Chromatography Technologies)

Solvent A: 0.05% TFA in water

Solvent B: 0.05% TFA in acetonitrile

Flow rate: 1 mL/min

Gradient: 20% to 80% B in 30 minutes

Column temp.: 30° C.

Detection: UV absorbance at 269 nm

### Method 2

Column: Acclaim OA

Solvent: 100 mM sodium sulfate titrated to pH 2.64 with methanesulfonic acid

Flow rate: 0.6 mL/min

Gradient: Isocratic

Column temp.: 30° C.

Detection: UV absorbance at 210 nm

Using Method 1, as described above, (R)-1-{2-[4′-(3-methoxy-propane-1-sulfonyl)-biphenyl-4-yl]-ethyl}-2-methyl-pyrrolidine had a retention time of 9.6 minutes. The concentration of the sample was determined by integrating the peak area and comparing it with the peak area of a reference standard.

Using Method 2, as described above, citric acid had a retention time of 4.5 min. And the concentration of the citric acid was determined by integrating the peak area at retention time 4.5 min and comparing it with the peak area of a pure reference standard of citric acid.

### Stoichiometry of (R)-1-{2-[4′-(3-Methoxy-propane-1-sulfonyl)-biphenyl-4-yl]-ethyl}-2-methyl-pyrrolidine mono-citrate (FORM1)

To determine the stoichiometry of citrate in (R)-1-{2-[4′-(3-methoxy-propane-1-sulfonyl)-biphenyl-4-yl]-ethyl}-2-methyl-pyrrolidine mono-citrate, two samples were weighed and assayed by each of the two methods as described above. The concentration of (R)-1-{2-[4′-(3-methoxy-propane-1-sulfonyl)-biphenyl-4-yl]-ethyl}-2-methyl-pyrrolidine determined using Method 1 was 1.61 mM/μg for both samples (see Table 8). The concentration of citric acid determined using Method 2 was 1.76 mM/μg for both samples (see Table 8). Thus, the stoichiometry was determined to be 1.09 (see Table 8). This result, within experimental error (10%), confirms the stoichiometry of (R)-1-{2-[4′-(3-methoxy-propane-1-sulfonyl)-biphenyl-4-yl]-ethyl}-2-methyl-pyrrolidine mono-citrate is 1 to 1.

### Stoichiometry of (R)-1-{2-[4′-(3-Methoxy-propane-1-sulfonyl)-biphenyl-4-yl]-ethyl}-2-methyl-pyrrolidine di-citrate

To determine the stoichiometry of citrate in (R)-1-{2-[4′-(3-methoxy-propane-1-sulfonyl)-biphenyl-4-yl]-ethyl}-2-methyl-pyrrolidine di-citrate, two samples were weighed and assayed by each of the two methods as described above. The concentration of (R)-1-{2-[4′-(3-methoxy-propane-1-sulfonyl)-biphenyl-4-yl]-ethyl}-2-methyl-pyrrolidine determined using Method 1 was 1.28 mM/μg for both samples (see Table 8). The concentration of citric acid determined using Method 2 was 2.47 mM/μg for Sample 1 and 2.43 mM/μg for Sample 2 (see Table 8). Thus, the stoichiometry was determined to be 1.93 for Sample 1 and 1.91 for Sample 2 (see Table 8). This result, within experimental error (10%), confirms the stoichiometry of (R)-1-{2-[4′-(3-methoxy-propane-1-sulfonyl)-biphenyl-4-yl]-ethyl}-2-methyl-pyrrolidine di-citrate is 1 to 2.

TABLE 8
Molar Ratios for (R)-1-{2-[4′-(3-Methoxy-propane-
1-sulfonyl)-biphenyl-4-yl]-ethyl}-2-methyl-pyrrolidine
Cmpd (I) and Citric Acid in the Mono- and Di-citrate salts

| | | | | Molar Ratio |
|---|---|---|---|---|
| | | mM Cmpd (I) | mM Citric Acid | (mM Citric |
| | Sample | per μg | per μg | Acid)/(mM |
| Sample | Prep # | Sample | Sample | Cmpd (I)) |
| Cmpd (I) | 1 | 1.61 | 1.76 | 1.09 |
| mono-citrate | 2 | 1.61 | 1.76 | 1.09 |
| Cmpd (I) | 1 | 1.28 | 2.47 | 1.93 |
| di-citrate | 2 | 1.28 | 2.43 | 1.91 |

### Example 8

### [³H] N-Alpha-Methyl-Histamine Competitive Histamine H3-Receptor Binding Assay

The histamine receptor binding assay was conducted using standard laboratory procedures as described below. A crude membrane fraction was prepared from whole rat brain cortex using a polytron to homogenize the tissue followed by differential centrifugation in a HEPES-based buffer containing protease inhibitors. Membranes where frozen at −80° C. until needed. Frozen membranes were thawed and resuspended in ice-cold assay buffer consisting of 50 mM TRIS containing 5 mM EDTA (pH=7.4). 50 micrograms (μg) of membrane protein was added to each well of a 96-well assay plate along with test compound and [³H]-N-α-methyl-histamine (1 nanomolar (nM) final assay concentration). Imetit was used as an assay positive control at varying concentrations. The plate was incubated for 30 min at room temperature. The assay was terminated by rapid filtration through a 96-well glass fiber filtration plate (GF/C) using a cell harvester (Perkin-Elmer). Captured membranes were washed three times with cold assay buffer and plates were dried at 50° C. 35 microliters (μL) of scintillation cocktail was added to each well and membrane-bound radioactivity was recorded using a TopCount 96-well plate scintillation counter (Perkin-Elmer).

The following table shows the observed activity for Compound (I) di-citrate of the present invention.

| Compound No. | Kᵢ Binding Assay (nM) |
|---|---|
| Cmpd (I) di-citrate | 0.75 |

### Example 9

### Human Histamine H3-Receptor Binding Assay—MDS Pharma Services (Taiwan)

Compounds were tested for their ability to bind to the human histamine H3-receptor using the MDS Pharma Services (Taiwan) assay, Catalogue No. 239810.

The following table shows the observed activity for Compound (I) of the present invention.

| Compound No. | Binding Assay (Ki, nM) |
|---|---|
| Cmpd (I) | 5.37 |

### Example 10

### Blockade of RAMH-Induced Drinking Assay

When administered to rodents, H3 agonists such as R-α-methyl-histamine (RAMH) induce a drinking response which is sensitive to reversal with an H3 antagonist. Blockade of RAMH-induced drinking can therefore be utilised as an in vivo assay for functional H3 antagonist activity. In this assay, male Sprague Dawley rats (250-350 g) were housed three per cage and maintained under a reverse 12 h light cycle (lights off at 1130 h). At 1030 h on the day of test, rats were individually housed in new cages and food was removed. 120 min later, rats were administered test article (vehicle or H3 antagonist, 0.3 mg/kg PO). 30 min later, water was removed, and RAMH (vehicle or RAMH 3 mg/kg salt SC) was administered. 10 min after administration of RAMH, weighed water bottles were placed in the cages, and drinking was allowed for 20 min. Water consumption was determined for each animal by weighing each bottle to the nearest 0.1 g. Data is expressed as percentage reduction in water intake according to the following formula:

[1−[(ANTAGONIST/RAMH)−(VEHICLE/RAMH)/(VEHICLE/RAMH)−(VEHICLE/VEHICLE)]]*100

| Compound No. | % inhibition of RAMH-induced drinking |
|---|---|
| Cmpd (I) di-citrate | 104 ± 14 |

Various modifications of the invention, in addition to those described herein, will be apparent to those skilled in the art from the foregoing description. Such modifications are also intended to fall within the scope of the appended claims. Each reference, including all patents, patent applications, and journal literature, cited in the present application is incorporated herein by reference in its entirety.

1. A compound selected from the group consisting of:
(R)-1-{2-[4′-(3-methoxy-propane-1-sulfonyl)-biphenyl-4-yl]-ethyl}-2-methyl-pyrrolidine mono-citrate;
(R)-1-{2-[4′-(3-methoxy-propane-1-sulfonyl)-biphenyl-4-yl]-ethyl}-2-methyl-pyrrolidine di-citrate;
(R)-1-{2-[4′-(3-methoxy-propane-1-sulfonyl)-biphenyl-4-yl]-ethyl}-2-methyl-pyrrolidine maleate; and
(R)-1-{2-[4′-(3-methoxy-propane-1-sulfonyl)-biphenyl-4-yl]-ethyl}-2-methyl-pyrrolidine hydrochloride.

2. The compound according to claim 1 that is (R)-1-{2-[4′-(3-methoxy-propane-1-sulfonyl)-biphenyl-4-yl]-ethyl}-2-methyl-pyrrolidine di-citrate.

3. The compound according to claim 2 having an X-ray diffraction pattern comprising a peak, expressed in terms of 2θ, at about 7.8°.

4. The compound according to claim 2 having an X-ray diffraction pattern comprising a peak, expressed in terms of 2θ, at about 10.3°.

5. The compound according to claim 2 having an X-ray diffraction pattern comprising a peak, expressed in terms of 2θ, at about 15.5°.

6. The compound according to claim 2 having an X-ray diffraction pattern comprising peaks, expressed in terms of 2θ, at about 7.8°, about 10.3°, and about 15.5°.

7. The compound according to claim 2 having an X-ray diffraction pattern comprising peaks, expressed in terms of 2θ, at about 7.8°, about 10.3°, about 11.8°, about 12.9°, about 13.6°, about 15.5°, about 18.1°, about 18.7°, about 19.7°, about 20.2°, about 22.0°, and about 23.2°.

8. The compound according to claim 2 having an X-ray diffraction pattern substantially as shown in FIG. 5.

9-58. (canceled)

59. The compound according to claim 2 having an infrared absorbance trace comprising peaks at about 1738 cm⁻¹, about 1726 cm⁻¹, and about 1686 cm⁻¹.

60. The compound according to claim 2 having an infrared absorbance trace comprising peaks at about 1738 cm⁻¹, about 1726 cm⁻¹, about 1686 cm⁻¹, about 1304 cm⁻¹, about 1213 cm⁻¹, and about 1146 cm⁻¹.

61. The compound according to claim 2 having an infrared absorbance trace substantially as shown in FIG. 7.

62. The compound according to claim 2 having a dynamic vapor sorption profile substantially as shown in FIG. 8.

63. The compound according to claim 2 having a differential scanning calorimetry trace comprising an endotherm at about 149° C.

64. The compound according to claim 2 having a differential scanning calorimetry trace comprising an endotherm with an extrapolated onset temperature between about 135° C. and about 155° C.

65. The compound according to claim 2 having a differential scanning calorimetry trace comprising an endotherm with an extrapolated onset temperature at about 145° C.

66. The compound according to claim 2 having a differential scanning calorimetry trace comprising an endotherm with a peak temperature between about 140° C. and about 160° C.

67. The compound according to claim 2 having a differential scanning calorimetry trace comprising an endotherm with an associated heat flow of about 106 joules per gram.

68. The compound according to claim 2 having a differential scanning calorimetry trace substantially as shown in FIG. 6.

69. The compound according to claim 2 having:
1) an X-ray diffraction pattern comprising peaks, expressed in terms of 2θ, at about 7.8°, about 10.3°, and about 15.5°;
2) a differential scanning calorimetry trace comprising an endotherm at about 149° C.; and
3) an infrared absorbance trace comprising peaks at about 1738 cm⁻¹, about 1726 cm⁻¹ and about 1686 cm⁻¹.

70. The compound according to claim 2 having:
1) an X-ray diffraction pattern comprising peaks, expressed in terms of 2θ, at about 7.8°, about 10.3°, about 11.8°, about 12.9°, about 13.6°, about 15.5°, about 18.1°, about 18.7°, about 19.7°, about 20.2°, about 22.0°, and about 23.2°;
2) a differential scanning calorimetry trace comprising an endotherm with an extrapolated onset temperature of about 145° C., a peak temperature of about 149° C., and an associated heat flow of about 106 joules per gram;
3) an infrared absorbance trace comprising peaks at about 1738 cm⁻¹, about 1726 cm⁻¹, about 1686 cm⁻¹, about 1304 cm⁻¹, about 1213 cm⁻¹, and about 1146 cm⁻¹; and
4) a dynamic vapor sorption profile substantially as shown in FIG. 8.

71. The compound according to claim 2 having an X-ray diffraction pattern comprising a peak, expressed in terms of 2θ, at about 7.7°.

72. The compound according to claim 2 having an X-ray diffraction pattern comprising a peak, expressed in terms of 2θ, at about 11.8°.

73. The compound according to claim 2 having an X-ray diffraction pattern comprising a peak, expressed in terms of 2θ, at about 18.7°.

74. The compound according to claim 2 having an X-ray diffraction pattern comprising peaks, expressed in terms of 2θ, at about 7.7°, about 11.8°, and about 18.7°.

75. The compound according to claim 2 having an X-ray diffraction pattern comprising peaks, expressed in terms of 2θ, at about 7.7°, about 10.3°, about 11.8°, about 12.9°, about 13.6°, about 15.4°, about 18.0°, about 18.7°, about 19.7°, about 20.2°, about 22.0°, and about 23.2°.

76. The compound according to claim 2 having an X-ray diffraction pattern substantially as shown in FIG. 16.

77. The compound according to claim 2 having a differential scanning calorimetry trace comprising an endotherm with an extrapolated onset temperature between about 140° C. and about 160° C.

78. The compound according to claim 2 having a differential scanning calorimetry trace comprising an endotherm with an extrapolated onset temperature at about 150° C.

79. The compound according to claim 2 having a differential scanning calorimetry trace comprising an endotherm with a peak temperature between about 145° C. and about 155° C.

80. The compound according to claim 2 having a differential scanning calorimetry trace comprising an endotherm with a peak temperature at about 151° C.

81. The compound according to claim 2 having a differential scanning calorimetry trace substantially as shown in FIG. 17.

82. The compound according to claim 2 having a Raman spectrum comprising peaks at about 746 cm⁻¹, about 1596 cm⁻¹, and about 2963 cm⁻¹.

83. The compound according to claim 2 having a Raman spectrum comprising peaks at about 416 cm⁻¹, about 746 cm⁻¹, about 788 cm⁻¹, about 1284 cm⁻¹, about 1596 cm⁻¹, about 1612 cm⁻¹, about 2963 cm⁻¹ and about 3073 cm⁻¹.

84. The compound according to claim 2 having a Raman spectrum substantially as shown in FIG. 19.

85. The compound according to claim 2 having:
1) an X-ray diffraction pattern comprising peaks, expressed in terms of 2θ, at about 7.7°, about 11.8°, and about 18.7°;
2) a differential scanning calorimetry trace comprising an endotherm with an extrapolated onset temperature at about 150° C. and a peak temperature at about 151° C.; and
3) a Raman spectrum comprising peaks at about 746 cm⁻¹, about 1596 cm⁻¹, and about 2963 cm⁻¹.

86. The compound according to claim 2 having:
1) an X-ray diffraction pattern comprising peaks, expressed in terms of 2θ, at about 7.7°, about 10.3°, about 11.8°, about 12.9°, about 13.6°, about 15.4°, about 18.0°, about 18.7°, about 19.7°, about 20.2°, about 22.0°, and about 23.2°;
2) a differential scanning calorimetry trace comprising an endotherm with an extrapolated onset temperature at about 150° C. and a peak temperature at about 151° C.; and
3) a Raman spectrum comprising peaks at about 416 cm⁻¹, about 746 cm⁻¹, about 788 cm⁻¹, about 1284 cm⁻¹, about 1596 cm⁻¹, about 1612 cm⁻¹, about 2963 cm⁻¹ and about 3073 cm⁻¹.

87. A composition comprising said compound according to claim 1.

88. A composition comprising said compound according to claim 2.

89. The composition according to claim 88, wherein said compound comprises about 50% or greater by weight of said composition.

90. The composition according to claim 88, wherein said compound comprises about 95% or greater by weight of said composition.

91. The composition according to claim 88, wherein said compound comprises about 99% or greater by weight of said composition.

92. A pharmaceutical composition comprising said compound according to claim 1 and a pharmaceutically acceptable carrier.

93. A pharmaceutical composition comprising said compound according to claim 2 and a pharmaceutically acceptable carrier.

94. A method for treating a histamine H3-receptor associated disorder in an individual comprising administering to said individual in need thereof a therapeutically effective amount of a compound according to claim 1.

95. The method according to claim 94, wherein said histamine H3-receptor associated disorder is selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, pain, dementia and Alzheimer's disease.

96. The method according to claim 94, wherein said histamine H3-receptor associated disorder is a disorder of sleep or wakefulness.

97. The method according to claim 94, wherein said histamine H3-receptor associated disorder is a cognitive disorder.

98. The method according to claim 94, wherein said histamine H3-receptor associated disorder is narcolepsy.

99. The method according to claim 94, wherein said histamine H3-receptor associated disorder is cataplexy.

100. A method of inducing wakefulness in an individual comprising administering to said individual in need thereof a therapeutically effective amount of a compound according to claim 1.

101. A method for treating a histamine H3-receptor associated disorder in an individual comprising administering to said individual in need thereof a therapeutically effective amount of a compound according to claim 2.

102. The method according to claim 101, wherein said histamine H3-receptor associated disorder is selected from the group consisting of a cognitive disorder, epilepsy, brain trauma, depression, obesity, disorders of sleep and wakefulness, narcolepsy, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, pain, dementia and Alzheimer's disease.

103. The method according to claim 101, wherein said histamine H3-receptor associated disorder is a disorder of sleep or wakefulness.

104. The method according to claim 101, wherein said histamine H3-receptor associated disorder is a cognitive disorder.

105. The method according to claim 101, wherein said histamine H3-receptor associated disorder is narcolepsy.

106. The method according to claim 101, wherein said histamine H3-receptor associated disorder is cataplexy.

107. A method of inducing wakefulness in an individual comprising administering to said individual in need thereof a therapeutically effective amount of a compound according to claim 2.

108. A process for preparing a pharmaceutical composition comprising admixing said compound according to claim 1 and a pharmaceutically acceptable carrier.

109. A process for preparing a pharmaceutical composition comprising admixing said compound according to claim 2 and a pharmaceutically acceptable carrier.
